(19) United States
(12) Patent Application Publication
(10) Pub. No.: US 2026/0132123 A1
(43) Pub. Date: May 14, 2026

(54) ABAD INHIBITOR COMPOUNDS

(71) Applicant: UNIVERSITY COURT OF THE UNIVERSITY OF ST ANDREWS, Fife (GB)

(72) Inventors: Laura AITKEN, Fife (GB); Frank GUNN-MOORE, Edinburgh (GB); Terry K SMITH, Fife (GB)

(21) Appl. No.: 18/686,238

(22) PCT Filed: Aug. 26, 2022
(86) PCT No.: PCT/GB2022/052204

§ 371 Date: Feb. 23, 2024

(30) Foreign Application Priority Data
Aug. 26, 2021 (GB) 2112240.3

(51) Int. Cl. C07D 403/04; A61K 31/167; A61K 31/277; A61K 31/40; A61K 31/4025; A61K 31/415; A61K 31/4155; A61K 31/416; A61K 31/417; A61K 31/4178; A61K 31/4192; A61K 31/4196; A61K 31/422; A61K 31/4245; A61K 31/426; A61K 31/427; A61K 31/433; A61K 31/437; A61K 31/44; A61K 31/4402; A61K 31/4406; A61K 31/4409; A61K 31/4439; A61K 31/454; A61K 31/4709; A61K 31/497; A61K 31/498; A61K 31/506; A61K 31/5377; A61P 35/00; C07C 233/88; C07C 255/25; C07D 207/08; C07D 213/38; C07D 213/64; C07D 231/12; C07D 233/64; C07D 233/90; C07D 249/04; C07D 249/08; C07D 277/28; C07D 401/06; C07D 401/10; C07D 401/12; C07D 403/06; C07D 403/12; C07D 405/06; C07D 405/12; C07D 409/04; C07D 409/06; C07D 409/12; C07D 409/14; C07D 413/06; C07D 413/12; C07D 417/06; C07D 471/04

(52) U.S. Cl. CPC C07D 403/04; A61K 31/167; A61K 31/277; A61K 31/40; A61K 31/4025; A61K 31/415; A61K 31/4155; A61K 31/416; A61K 31/417; A61K 31/4178; A61K 31/4192; A61K 31/4196; A61K 31/422; A61K 31/4245; A61K 31/426; A61K 31/427; A61K 31/433; A61K 31/437; A61K 31/44; A61K 31/4402; A61K 31/4406; A61K 31/4409; A61K 31/4439; A61K 31/454; A61K 31/4709; A61K 31/497; A61K 31/498; A61K 31/506; A61K 31/5377; A61P 35/00; C07C 233/88; C07C 255/25; C07D 207/08; C07D 213/38; C07D 213/64; C07D 231/12; C07D 233/64; C07D 233/90; C07D 249/04; C07D 249/08; C07D 277/28; C07D 401/06; C07D 401/10; C07D 401/12; C07D 403/06; C07D 403/12; C07D 405/06; C07D 405/12; C07D 409/04; C07D 409/06; C07D 409/12; C07D 409/14; C07D 413/06; C07D 413/12; C07D 417/06; C07D 471/04

(57) ABSTRACT

The present invention concerns Amyloid Binding Alcohol Dehydrogenase (ABAD) inhibitors (I) or (II) that interact non-competitively with nicotinamide adenine dinucleotide+H (NADH). Inhibition of ABAD is useful in the treatment or prophylaxis of disease, including Alzheimer's disease (AD) and cancer. Accordingly, the present invention also concerns ABAD inhibitors for use as a medicament, specifically for use in the treatment of Alzheimer's disease and/or cancer.

### FIELD OF THE INVENTION

The present invention concerns Amyloid Binding Alcohol Dehydrogenase (ABAD) inhibitors that interact non-competitively with nicotinamide adenine dinucleotide+H (NADH). Inhibition of ABAD is useful in the treatment or prophylaxis of disease, including Alzheimer's disease (AD) and cancer. Accordingly, the present invention also concerns ABAD inhibitors for use as a medicament, specifically for use in the treatment of Alzheimer's disease and/or cancer.

### BACKGROUND OF THE INVENTION

Dementia is a neurological disease, which is currently estimated to affect over 850,000 people in the UK, with a cost to the economy of over £26 billion (current figures from www.dementiastatistics.org). At present there are no disease modifying therapies available to treat dementia. Rather, current treatments act to slow down the progression of the disease without treating the underlying causes.

Changes in the way our bodies produce energy are well reported in AD. As a person ages, the energy sources used by the person's brain use can switch from solely using glucose to utilising other sources. In AD, glucose metabolism is significantly decreased and stored fat is relied upon for energy production. The main function of ABAD, also known as 17β-hydroxysteroid type 10 (17β-HSD10), is to produce energy for the brain, and it does so via β-fatty acid oxidation when glucose levels are low, as is the case in AD. Energy production requires the correct functioning of structures within nerve cells but in the early stages of AD, such structures do not function efficiently.

This abnormality, called mitochondrial dysfunction, can be caused by a build-up of amyloid (a hallmark of AD) within mitochondria, allowing amyloid to attach to ABAD. This does not occur in normal aging. When amyloid attaches to ABAD, it alters its function and causes a build-up of toxins and the activation of specific genes, all of which results in damaging changes in synapses, which impairs the memory capabilities of the brain.

ABAD was first identified as an amyloid beta peptide (Aβ) binding protein in 1997 (S. D. Yan et al. Nature, 1997, 389, 689-695). This finding was subsequently confirmed in a number of later studies (see, for example, J. W. Lustbader et al., Science, 2004, 304 (5669), 448-452 and J. Yao et al., Mol. Cell. Neurosci, 2007, 35 (2), 377-382). ABAD is known to interact with the two major plaque forming isoforms of Aβ, namely Aβ(1-40) and Aβ(1-42), leading to distortion of the enzyme structure and inhibition of its normal function as an energy provider for cells (see U. C. Oppermann et al., FEBS lett., 1999, 451 (3), 238-242 and S. D. Yan et al., J. Biol. Chem., 1999, 274, 2145-2156). In vitro experiments have shown that the interaction between ABAD and Aβ is cytotoxic and ABAD's function is diminished resulting in a build-up of reactive oxygen species (ROS) and toxins, leading to mitochondrial dysfunction.

Using site directed mutagenesis and surface plasmon resonance, Lustbader et al. (2004, supra) identified the binding site for Aβ as the LD loop of ABAD, and subsequently synthesised a 28-amino acid peptide to mimic this region. This peptide is termed the 17β-HSD10-decoy peptide, or 17β-HSD10-DP. It was shown using surface plasmon resonance that 7β-HSD10-DP can bind to Aβ(1-40) and Aβ(1-42), thereby inhibiting their binding to ABAD. This translated into a cytoprotective effect in cell culture experiments: cultured wild type cortical neurons exposed to Aβ(1-42) showed a significant increase in cell death, as measured by cytochrome-c release, whilst those pre-incubated with 17β-HSD10-DP did not.

Direct modulation of ABAD activity is also reported to be effective in treating AD. In vitro experiments with SH-SY5Y (neuroblastoma cells) administered with the ABAD inhibitor, AG18051, show a reduction in mitochondrial dysfunction and oxidative stress associated with the interaction between ABAD and Aβ, and protect cultured SH-SY5Y cells from Aβ mediated cytotoxicity (Lim et al., PLoS One, 2011, 6 (12), e28887).

Not only is ABAD an important target in AD, it has also been implicated in various forms of cancer, such as prostate cancer and notably in cases deemed castration resistant prostate cancer (CRPC). See P. R. Dillard, M. F. Lin and S. A. Khan, Mol. Cel. Endocrinol., 2008, 295 (1-2), 115-120; E. Jernberg et al., PLoS One, 2013, 8 (11), e77407; and E. Carlson et al., BMC Cancer, 2015, 15:166 for more details. Prostate cancer has the second highest mortality rate and accounts for approximately 29% of cancer cases in men, but the underlying causes for the disease are yet to be established. Prostate cancer is a hormone sensitive cancer, where androgens, namely testosterone and dihydrotestosterone (DHT), play a pivotal role in its development and progression. Testosterone and DHT promote cancer cell growth through their interaction with androgen receptors within the prostate (A. Zhang et al., Horm. Cancer, 2016, 7 (2), 104-113). Current treatments of prostate cancer typically focus on reducing testosterone and DHT levels through chemical or surgical castration. However, such treatment is limited in patients suffering from metastatic CRPC. In these cases, levels of ABAD are significantly increased. ABAD is reported to oxidise the DHT metabolite 5α-androstane-3,17-diol (3α-androstanediol or 3α-diol) back to DHT, thereby generating significantly increased levels of DHT (Zhang et al., 2016, supra). High amounts of DHT can increase the growth of prostate cancer and make it more difficult to treat.

The present disclosure provides alternative ABAD inhibitors useful in the treatment or prophylaxis of conditions such as those disclosed herein. The ABAD inhibitors disclosed herein are especially useful in the treatment or prophylaxis of AD and prostate cancer (CRPC in particular).

### SUMMARY OF THE INVENTION

The inventors have found that the compounds disclosed herein are surprisingly effective ABAD inhibitors. Subsequently, the compounds disclosed herein have the potential to reverse memory deficits attributed to amyloid-ABAD interactions. The compounds disclosed herein interact non-competitively with NADH, offering a potentially greater enzyme specificity than some known ABAD inhibitors.

The skilled person is aware that any reference to an aspect of the current disclosure includes that aspect and/or any embodiment of that aspect. For example, any reference to the first aspect includes the first aspect and/or any embodiment of the first aspect.

Viewed from a first aspect, there is provided a compound of formula (I) or (II):

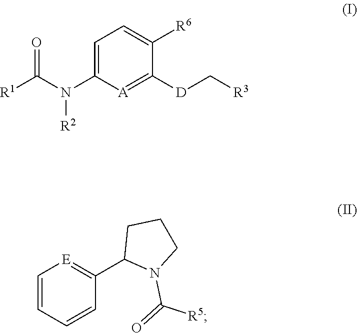

wherein R¹ is any one selected from the group consisting of C₆-C₁₀aryl, C₃-C₅heteroaryl, C₁₀-C₁₆biaryl, C₆-C₁₄biheteroaryl, C₅-C₈cycloalkyl, C₃-C₇heterocycloalkyl, C₁-C₆alkyl, C₆-C₁₀arylC₁-C₃alkyl, C₁₀-C₁₆biarylC₁-C₃alkyl and C₃-C₈cycloalkylC₁-C₃alkyl optionally substituted one or more times with any one or a combination selected from the group consisting of halo, C₁-C₄haloalkyl, C₁-C₄alkyl, C₁-C₄alkoxy, C₁-C₄haloalkoxy, hydroxy and methylsulfonyl;
R² is any one selected from the group consisting of C₁-C₄alkyl, C₃-C₅cycloalkyl, C₃-C₅cycloalkylC₁-C₃alkyl, C₁-C₄alkylether, and di(C₁₋₄alkyl)aminoC₁₋₃alkyl;
A is CH or N;
R³ is any one selected from the group consisting of C₂-C₅heteroaryl optionally substituted one or more times with any one or a combination selected from the group consisting of C₁-C₄alkyl, C₃-C₅cycloalkyl, halo, hydroxy and C₁₋₃alkoxy; ethyne, hydroxymethyl, amido, N-methylamido and cyano;
D is NR⁷ or O, wherein R⁷ is C₁₋₃alkyl or C₁₋₃alkylol;
R⁶ is H; or
R⁶ and D together form a 5 or 6 membered heterocycle optionally substituted one or more times with any one or a combination selected from the group consisting of oxo, halo, C₁₋₃alkyl, C₁₋₃haloalkyl, hydroxy and C₁₋₃alkoxy;
E is CR⁴ or N;
R⁴ is any one selected from the group consisting of C₆aryl, C₃-C₇heteroaryl, C₆-C₁₄biheteroaryl, H, halo, C₁₋₆alkyl, C₁₋₆haloalkyl, C₁₋₆alkoxy, wherein the C₆aryl, C₃-C₇heteroaryl and C₆-C₁₄biheteroaryl are optionally substituted one or more times with any one or a combination selected from the group consisting of hydroxy, C₁-C₄alkoxy, C₁-C₄haloalkoxy, halo, C₁-C₄haloalkyl, C₁-C₄alkyl, C₃-C₅cycloalkyl, C₄-C₅heterocycloalkyl, C₁-C₄alkanol, cyano, methane sulfonate and dimethylamido; and
R⁵ is any one selected from the group consisting of C₂-C₅heteroaryl, C₆aryl, C₃₋₅cycloalkyl and C₁₋₄alkyl, wherein the C₂-C₅heteroaryl, C₆aryl and C₃₋₅cycloalkyl are each optionally substituted one or more times with any one or a combination selected from the group consisting of halo and C₁-C₄alkyl.

Viewed from a second aspect, there is provided a composition comprising the compound of the first aspect and one or more pharmaceutically acceptable excipients.

Viewed from a third aspect, there is provided a compound of the first aspect or a composition of the second aspect for use as an ABAD inhibitor.

Viewed from a fourth aspect, there is provided a compound of the first aspect or a composition of the second aspect for use as a medicament.

Viewed from a fifth aspect, there is provided a compound of the first aspect or a composition of the second aspect for use in a method of treatment or prophylaxis of dementia or cancer.

Viewed from a sixth aspect, there is provided a method of inhibiting ABAD activity in a subject, the method comprising administering an effective amount of a compound of the first aspect or a composition of the second aspect.

Viewed from a seventh aspect, there is provided a method of treatment comprising administering an effective amount of a compound of the first aspect or a composition of the second aspect to a subject.

Viewed from an eighth aspect, there is provided a method for the treatment or prophylaxis of dementia or cancer, the method comprising administering an effective amount of a compound of the first aspect or a composition of the second aspect to a subject.

### BRIEF DESCRIPTION OF THE FIGURES

FIG. 1: Chemical modifications made to the basic cluster backbone.

FIG. 2: Box plot summarising compound type versus ABAD/17β-HSD10 activity.

FIG. 3: Key interactions between 17β-HSD10 and ESC1002033. A) H-bond from the imidazole to GLN162 and also a H-bond to a well-ordered water molecule with an edge to face interaction between the central aryl ring and TYR 168 of the catalytic triad. B) A small hydrophobic pocket close to the imidazole and water.

FIG. 4: Top: Indicates the previously published AG18051, its interaction with ABAD/17β-HSD10 (ABAD) and (bottom left) the key residues for interaction are defined as GLN162, GLN165 and TYR168. Bottom right is a superposition of ESC1002033 over bottom left.

FIG. 5: A) Targeting the edge to face interaction at Tyrosine 168; B) targeting Leucine 205.

FIG. 6: Confirmation of an inactive singleton enantiomer.

FIG. 7: Crystal structures depicting the key interactions of ESC1002421 and 17β-HSD10.

FIG. 8: A summary of lipophilicity vs potency improvements of the singleton series.

FIG. 9: Thermal shift data for ESC1002033 and ESC1002082 giving representative examples of the derivative plots and showing the correlation between pIC₅₀ and ΔTₘ.

FIG. 10: 17β-HSD10 activity in HEK293 mtsABAD cell line using the fluorogenic probe-(-)CHANA (Values shown are mean n=6±SEM).

FIG. 11: Example electron density map, with initial electron density mFo-DFc in green on the left (ligand superimposed for reference) and the 2mFo-Dfc density for the fitted ligand and cofactor on the right.

FIG. 12: ESC1002033 bound in a deep cleft with some notable features in its conformation. Binding site shown in (A) and surface (B).

FIG. 13: A) ESC1002332 bound in same site as ESC1002033. B) Differences in ESC1002332 and ESC1002033 binding.

### DETAILED DESCRIPTION OF THE INVENTION

The inventors have found that the compounds disclosed herein are surprisingly effective ABAD inhibitors, with some of the inhibitors exhibiting IC₅₀ values of less than 100 nM. The compounds disclosed herein interact non-competitively with NADH, offering potentially greater enzyme specificity than some known ABAD inhibitors. The compounds are now disclosed in detail.

In the discussion that follows, reference is made to a number of terms, which have the meanings provided below, unless a context indicates to the contrary. The nomenclature used herein for defining compounds, in particular the compounds according to the invention, is in general based on the rules of the IUPAC organisation for chemical compounds, specifically the “IUPAC Compendium of Chemical Terminology (Gold Book)”. For the avoidance of doubt, if a rule of the IUPAC organisation is contrary to a definition provided herein, the definition herein is to prevail. Furthermore, if a compound structure is contrary to the name provided for the structure, the structure is to prevail.

The term “aromatic” defines a cyclically conjugated molecular entity with a stability (due to delocalisation) significantly greater than that of a hypothetical localised structure. The Hückel rule is often used in the art to assess aromatic character; monocyclic planar (or almost planar) systems of trigonally (or sometimes diagonally) hybridised atoms that contain (4n+2) π-electrons (where n is a non-negative integer) will exhibit aromatic character. The rule is generally limited to n=0 to 5.

The term “conjugated” or variants thereof defines a molecular entity whose structure may be represented as a system of alternating single and multiple bonds. In such systems, conjugation is the interaction of one p-orbital with another across an intervening π-bond in such structures. In appropriate molecular entities d-orbitals may be involved. The term is also extended to the analogous interaction involving a p-orbital containing an unshared electron pair.

The term “delocalised” defines the π-bonding in a conjugated system where the bonding is not localised between two atoms, but instead each link has a fractional double bond character, or bond order.

The term “heteroaromatic” defines a cyclically conjugated molecular entity comprising heteroatoms, with a stability (due to delocalisation) significantly greater than that of a hypothetical localised structure.

The term “cyclic” or variants thereof defines a compound in which one or more series of atoms in the compound is connected to form a ring. Whereas, the term “acyclic” defines a compound containing no rings of atoms.

The term “aryl” defines a group derived from an arene by removal of a hydrogen atom from a ring carbon atom, wherein an arene is a monocyclic or polycyclic aromatic hydrocarbon.

The term “heteroaryl” defines a group derived from a heteroarene by removal of a hydrogen atom from a ring carbon or heteroatom, wherein a heteroarene is a monocyclic or polycyclic aromatic hydrocarbon comprising one or more heteroatoms.

The term “biaryl” defines a group derived from a biarene by removal of a hydrogen atom from a ring carbon atom, wherein a biarene is an assembly of two aryl groups, joined by a single bond.

The term “biheteroaryl” defines a group derived from a biheteroarene by removal of a hydrogen atom from a ring carbon or heteroatom, wherein a biheteroarene is an assembly of two heteroaryl groups, joined by a single bond.

The term “cycloalkyl” defines all univalent groups derived from cycloalkanes by removal of a hydrogen atom from a ring carbon atom. The term “cycloalkane” defines saturated monocyclic and polycyclic hydrocarbons. C₃-C₅cycloalkyl consists of cyclopropyl, cyclobutyl and cyclopentyl; and C₅-C₈cycloalkyl consists of cyclopentyl, cyclohexyl, cycloheptyl and cyclooctyl.

The term “heterocycloalkyl” defines all univalent groups derived from heterocycloalkanes by removal of a hydrogen atom from a ring carbon atom or heteroatom. The term “heterocycloalkane” defines monocyclic and polycyclic hydrocarbons comprising heteroatoms. C₃-C₇heterocycloalkyl may correspond to cyclopentyl, cyclohexyl, cycloheptyl and cyclooctyl, wherein at least one carbon atom is replaced with a heteroatom. In order to satisfy covalency, the heteroatom may be bound to one or more hydrogen atoms.

The term “heterocycle” defines all cyclic compounds comprising atoms of at least two different elements in the ring. For the avoidance of doubt, heterocycloalkanes and heteroarenes are included within the term “heterocycle”.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof will be understood to imply the inclusion of a stated element, integer or step, or group of elements, integers or steps, and the exclusion of any other element, integer or step or group of elements, integers or steps.

The term “alkyl” is well known in the art and defines univalent groups derived from alkanes by removal of a hydrogen atom from any carbon atom, wherein the term “alkane” is intended to define cyclic or acyclic branched or unbranched hydrocarbons having the general formula CₙH₂ₙ₊₂, wherein n is an integer ≥1. C₁-C₄alkyl consists of methyl, ethyl, n-propyl, iso-propyl, n-butyl, sec-butyl, iso-butyl and tert-butyl.

The term “halo” is well known in the art and defines a halogen radical that, when bonded to a carbon radical makes a fluoride, chloride, bromide or iodide compound.

The term “haloalkyl” defines an alkyl in which one or more hydrogen atoms have been replaced with a halo.

The term “alkyloxy” is synonymous with “alkoxy” and when used herein defines a univalent group comprising an alkyl singly bonded to an oxygen atom, derived from the corresponding alcohol by removal of the hydrogen atom bonded to the oxygen atom.

The term “haloalkoxy” defines an alkoxy in which one or more hydrogen atoms have been replaced with a halo.

The term “alkylether” defines a univalent group derived from an ether group (an oxygen atom connected to two alkyl groups), by removal of a hydrogen atom from any carbon atom.

The term “alkanol” defines a univalent group comprising an alkylene singly bonded to a hydroxy group, derived from the corresponding alcohol by removal of a hydrogen atom bonded to a carbon atom.

The term “treatment” defines the therapeutic treatment of a subject that may be a human or non-human animal, in order to impede or reduce or halt the rate of progress of a condition, or to ameliorate or cure the condition. Prophylaxis of the condition as a result of treatment is also included. References to prophylaxis are intended herein not to require complete prevention of a condition: its development may instead be hindered through treatment in accordance with the invention.

By an “effective amount” herein defines an amount of any one or a combination of the compounds described herein that is sufficient to impede a condition and thus produces the desired therapeutic or inhibitory effect.

The term “stereoisomer” is used herein to refer to isomers that possess identical molecular formulae and sequence of bonded atoms, but which differ in the arrangement of their atoms in space.

The term “enantiomer” defines one of a pair of molecular entities that are mirror images of each other and non-superimposable, i.e. cannot be brought into coincidence by translation and rigid rotation transformations. Enantiomers are chiral molecules, i.e. are distinguishable from their mirror image.

The term “racemic” is used herein to pertain to a racemate. A racemate defines a substantially equimolar mixture of a pair of enantiomers.

The term “diastereoisomers” (also known as diastereomers) defines stereoisomers that are not related as mirror images.

The term “solvate” is used herein to refer to a complex comprising a solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

The term “isotope” is used herein to define a variant of a particular chemical element, in which the nucleus necessarily has the same atomic number but has a different mass number owing to it possessing a different number of neutrons.

The term “prodrug” is used herein to refer to a compound which acts as a drug precursor and which, upon administration to a subject, undergoes conversion by metabolic or other chemical processes to yield a compound disclosed herein.

The term “pharmaceutically acceptable excipient” defines substances other than a pharmacologically active drug or prodrug, which are included in a pharmaceutical product.

The term “enteral” is used to refer to administration of a compound through the gastrointestinal tract. Enteral administration may be oral administration, i.e. administration through the mouth.

The term “parenteral” is used to refer to administration of a compound into the body via means other than the gastrointestinal tract. Parenteral administration includes intravenous administration (directly into a vein), intramuscular administration (into the muscle), intradermal administration (beneath the skin) or subcutaneous administration (into the fat or skin). Parenteral administration may be carried out via a bolus injection, in which a discrete amount of compound is administered in one injection.

As described above, in a first aspect, there is provided a compound of formula (I) or (II):

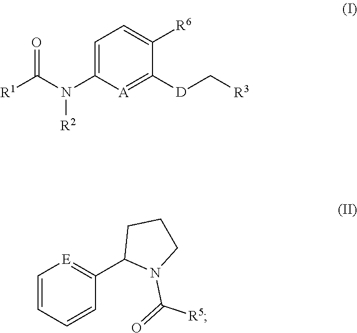

wherein R¹ is any one selected from the group consisting of C₆-C₁₀aryl, C₃-C₅heteroaryl, C₁₂biaryl, C₆-C₁₀biheteroaryl, C₅-C₈cycloalkyl, C₃-C₇heterocycloalkyl, C₁-C₆alkyl, C₆-C₁₀arylC₁-C₃alkyl, C₁₂biarylC₁-C₃alkyl and C₅-C₈cycloalkylC₁-C₃alkyl optionally substituted one or more times with any one or a combination selected from the group consisting of halo, C₁-C₄haloalkyl, C₁-C₄alkyl, C₁-C₄alkoxy, hydroxy and methylsulfonyl;
R² is any one selected from the group consisting of C₁-C₄alkyl, C₃-C₅cycloalkyl, C₃-C₅cycloalkylC₁-C₃alkyl, C₁-C₄alkylether, and di(C₁₋₄alkyl)aminoC₁₋₃alkyl;
A is CH or N;
R³ is any one selected from the group consisting of C₂-C₅heteroaryl optionally substituted one or more times with any one or a combination selected from the group consisting of C₁-C₄alkyl, C₃-C₅cycloalkyl, halo, hydroxy and C₁₋₃alkoxy; ethyne, hydroxymethyl, amido, N-methylamido and cyano;
D is NR⁷ or O, wherein R⁷ is C₁₋₃alkyl or C₁₋₃alkylol;
R⁶ is H; or
R⁶ and D together form a 5 or 6 membered heterocycle optionally substituted one or more times with any one or a combination selected from the group consisting of oxo, halo, C₁₋₃alkyl, C₁₋₃haloalkyl, hydroxy and C₁₋₃alkoxy;
E is CR⁴ or N;
R⁴ is any one selected from the group consisting of C₆aryl, C₃-C₇heteroaryl, C₆-C₁₄biheteroaryl, H, halo, C₁₋₆alkyl, C₁₋₆haloalkyl, C₁₋₆alkoxy, wherein the C₆aryl, C₃-C₇heteroaryl and C₆-C₁₄biheteroaryl are optionally substituted one or more times with any one or a combination selected from the group consisting of hydroxy, C₁-C₄alkoxy, C₁-C₄haloalkoxy, halo, C₁-C₄haloalkyl, C₁-C₄alkyl, C₃-C₅cycloalkyl, C₄-C₅heterocycloalkyl, C₁-C₄alkanol, cyano and methane sulfonato; and
R⁵ is C₂-C₅heteroaryl, optionally substituted one or more times with any one or a combination selected from the group consisting of halo and C₁-C₄alkyl.

Compounds of formula (I) are also referred to herein as “cluster compounds”. R¹ of such cluster compounds is any one selected from the group consisting of C₆-C₁₀aryl, C₃-C₅heteroaryl, C₁₂biaryl, C₆-C₁₀biheteroaryl, C₅-C₈cycloalkyl, C₃-C₇heterocycloalkyl, C₁-C₆alkyl (such as C₃₋₆alkyl), C₆-C₁₀arylC₁-C₃alkyl, C₁₂biarylC₁-C₃alkyl and C₅-C₈cycloalkylC₁-C₃alkyl, each optionally substituted one or more times with any one or a combination selected from the group consisting of halo, C₁-C₄haloalkyl, C₁-C₄alkyl, C₁-C₄alkoxy, hydroxy and methylsulfonyl.

C₆-C₁₀aryl includes phenyl, naphthalenyl, cyclooctatetraenyl and cyclodecapentaenyl. In some embodiments, C₆-C₁₀aryl is phenyl or naphthalenyl, such as phenyl.

C₃-C₅heteroaryl includes thiophenyl, pyridyl, pyrimidinyl, pyrazinyl, imidazolyl, pyrazolyl, thiazolyl and triazolyl. In some embodiments, the C₃-C₅heteroaryl of R¹ is thiophenyl, pyridyl, pyrimidinyl or pyrazinyl. For example, the C₃-C₅heteroaryl of R¹ may be thienyl or pyridyl.

C₁₂biaryl may be biphenyl.

C₆-C₁₀biheteroaryl includes bithienyl, bipyridyl, thienylpyridyl, thienylpyrimidinyl, pyridylpyrimidinyl, thienylpyrazinyl, pyridylpyrazinyl, bipyrimidinyl, bipyrazinyl and pyrimidinylpyrazinyl. In some embodiments, C₆-C₁₀biheteroaryl is bithienyl or bipyridyl.

C₅-C₈cycloalkyl includes cyclopentyl, cyclohexyl, cycloheptyl and cyclooctyl. In some embodiments, C₅-C₈cycloalkyl is cyclopentyl or cyclohexyl, such as cyclohexyl.

C₃-C₇heterocycloalkyl includes morpholino, tetrahydropyranyl, piperidinyl, tetrahydrofuranyl, pyrrolidinyl, azepinyl and diazepinyl. In some embodiments, C₃-C₇heterocycloalkyl is morpholino, tetrahydropyranyl or piperidinyl.

C₁-C₆alkyl includes methyl, ethyl, n-propyl, iso-propyl, n-butyl, sec-butyl, tert-butyl, n-pentyl, 2-methylbutan-2-yl, 2,2-dimethylpropyl, 3-methylbutyl, pentan-2-yl, pentan-3-yl, 3-methylbutan-2-yl, 2-methylbutyl, n-hexane, hexan-2-yl, hexan-3-yl, 4-methylpentanyl, 4-methylpentan-2-yl, 2-methylpentan-2-yl and 2-methylpentanyl. In some embodiments, C₁₋₆alkyl is C₃₋₆alkyl. In some embodiments, C₃-C₆alkyl is n-propyl or iso-propyl, such as iso-propyl.

C₆-C₁₀arylC₁-C₃alkyl includes phenylC₁-C₃alkyl, naphthalenylC₁-C₃alkyl, cyclooctatetraenylC₁-C₃alkyl and cyclodecapentaenylC₁-C₃alkyl. In some embodiments, C₆-C₁₀arylC₁-C₃alkyl is selected from the group consisting of phenylmethyl, phenylethyl, phenylpropyl, naphthalenylmethyl, naphthalenylethyl and naphthalenylpropyl. For example, C₆-C₁₀arylC₁-C₃alkyl may be phenylmethyl, phenylethyl or phenylpropyl.

C₁₂biarylC₁-C₃alkyl includes biphenylC₁-C₃alkyl. In some embodiments, C₁₂biarylC₁-C₃alkyl is biphenylmethyl, biphenylethyl or biphenylpropyl.

C₅-C₈cycloalkylC₁-C₃alkyl includes cyclopentylC₁-C₃alkyl, cyclohexylC₁-C₃alkyl, cycloheptylC₁-C₃alkyl and cyclooctylC₁-C₃alkyl. In some embodiments, C₅-C₈cycloalkylC₁-C₃alkyl is selected from the group consisting of cyclopentylmethyl, cyclopentylethyl, cyclopentylpropyl, cyclohexylmethyl, cyclohexylethyl and cyclohexylpropyl.

As described above, the C₆-C₁₀aryl, C₃-C₅heteroaryl, C₁₂biaryl, C₆-C₁₀biheteroaryl, C₅-C₈cycloalkyl, C₃-C₇heterocycloalkyl, C₃-C₆alkyl, C₆-C₁₀arylC₁-C₃alkyl, C₁₂biarylC₁-C₃alkyl and C₅-C₈cycloalkylC₁-C₃alkyl may each be substituted one or more times with any one or a combination selected from the group consisting of halo, C₁-C₄haloalkyl, C₁-C₄alkyl, C₁-C₄alkoxy, hydroxy (—OH) and methylsulfonyl (—SO₂CH₃).

In some embodiments, the halo substituent is fluoro or chloro, e.g. fluoro.

The C₁-C₄haloalkyl is any one or a combination selected from the group consisting of halomethyl, haloethyl, halo-n-propyl, halo-iso-propyl, halo-n-butyl, halo-sec-butyl, halo-iso-butyl and halo-tert-butyl. In some embodiments, the halo of the C₁-C₄haloalkyl is fluoro. In some embodiments, the C₁-C₄haloalkyl is any one or a combination selected from the group consisting of trifluoromethyl, difluoromethyl and monofluoromethyl, such as trifluoromethyl.

In some embodiments, the C₁-C₄alkyl substituent is any one or a combination selected from methyl and ethyl.

The C₁-C₄alkoxy substituent is any one or a combination selected from the group consisting of methoxy, ethoxy, n-propoxy, iso-propoxy, n-butoxy, sec-butoxy, iso-butoxy and tert-butoxy. In some embodiments, the C₁-C₄alkoxy is any one or a combination selected from the group consisting of methoxy and ethoxy, such as methoxy.

In some embodiments, R¹ is any one selected from the group consisting of C₆-C₁₀aryl, C₃-C₅heteroaryl, C₁₂biaryl, C₅-C₈cycloalkyl, C₃-C₆alkyl and C₆-C₁₀arylC₁-C₃alkyl, each optionally substituted one or more times with any one or a combination selected from the group consisting of halo, C₁-C₄haloalkyl, C₁-C₄alkyl, C₁-C₄alkoxy, hydroxy and methylsulfonyl. In such embodiments, R¹ is not an optionally substituted C₆-C₁₀biheteroaryl, C₃-C₇heterocycloalkyl, C₁₂biarylC₁-C₃alkyl or C₅-C₈cycloalkylC₁-C₃alkyl.

In some embodiments, R¹ is any one selected from the group consisting of C₆-C₁₀aryl, C₃-C₅heteroaryl, C₅-C₈cycloalkyl and C₃-C₆alkyl, each optionally substituted one or more times with any one or a combination selected from the group consisting of halo, C₁-C₄haloalkyl, C₁-C₄alkyl, C₁-C₄alkoxy and hydroxy and methylsulfonyl. In such embodiments, R¹ is not an optionally substituted C₁₂biaryl, C₆-C₁₀biheteroaryl, C₃-C₇heterocycloalkyl, C₆-C₁₀arylC₁-C₃alkyl, C₁₂biarylC₁-C₃alkyl or C₅-C₈cycloalkylC₁-C₃alkyl.

In some embodiments, R¹ is any one selected from the group consisting of phenyl, thiophenyl, pyridinyl, cyclohexyl and iso-propyl, each optionally substituted one or more times with any one or a combination selected from the group consisting of halo, C₁-C₄haloalkyl, C₁-C₄alkyl, C₁-C₄alkoxy, hydroxy and methylsulfonyl.

In some embodiments, the optional substituents of R¹ are selected from the group consisting of halo, C₁-C₄haloalkyl, C₁-C₄alkyl, C₁-C₄alkoxy and hydroxy (i.e. the optional substituents are not methylsulfonyl). In some embodiments, the optional substituents are selected from the group consisting of fluoro, chloro, trifluoromethyl, methyl, ethyl, methoxy and hydroxy. For example, the optional substituents may be selected from the group consisting of fluoro, chloro, trifluoromethyl, methyl, ethyl and methoxy.

In particular embodiments, R¹ is any one selected from the group consisting of phenyl, thiophenyl, pyridinyl, cyclohexyl and iso-propyl, each optionally substituted one or more times with any one or a combination selected from the group consisting of fluoro, chloro, trifluoromethyl, methyl, ethyl and methoxy.

As described above, R² is any one selected from the group consisting of C₁-C₄alkyl, C₃-C₅cycloalkyl, C₃-C₅cycloalkylC₁-C₃alkyl, C₁-C₄alkylether and di(C₁₋₄alkyl)aminoC₁₋₃alkyl.

In some embodiments, the C₁-C₄alkyl is iso-butyl, iso-propyl, ethyl, n-butyl or dimethylpropyl.

C₃-C₅cycloalkyl includes cyclopropyl, cyclobutyl and cyclopentyl. In some embodiments, the C₃-C₅cycloalkyl is cyclobutyl or cyclopropyl.

C₃-C₅cycloalkylC₁-C₃alkyl includes cyclopropylC₁-C₃alkyl, cyclobutylC₁-C₃alkyl and cyclopentylC₁-C₃alkyl. In some embodiments, the C₃-C₅cycloalkylC₁-C₃alkyl is selected from the group consisting of cyclobutylmethyl, cyclopropylmethyl, cyclobutylethyl and cyclopropylethyl. For example, the C₃-C₅cycloalkylC₁-C₃alkyl may be cyclobutylmethyl or cyclopropylmethyl.

In some embodiments, the C₁-C₄alkylether is methoxyethyl or isopropoxyethyl.

In some embodiments, the di(C₁₋₄alkyl)aminoC₁₋₃alkyl is dimethylaminoC₁₋₃alkyl, such as dimethylaminoethyl.

In some embodiments, R² is any one selected from the group consisting of C₁-C₄alkyl, C₃-C₅cycloalkyl, C₃-C₅cycloalkylC₁-C₃alkyl, and C₁-C₄alkylether.

In some embodiments, R² is any one selected from the group consisting of C₁-C₄alkyl and C₃-C₅cycloalkylC₁-C₃alkyl. For example, R² may be any one selected from the group consisting of iso-butyl, iso-propyl, ethyl, n-butyl, dimethylpropyl, cyclobutylmethyl and cyclopropylmethyl. In some embodiments, R² is iso-butyl.

As described above, A is CH or N. When A is CH, the central ring of formula (I) may be a benzene ring, and when A is a nitrogen atom, the central ring of formula (I) may be a pyridine ring. In some embodiments, A is CH.

As described above, R³ is any one selected from the group consisting of C₂-C₅heteroaryl optionally substituted one or more times with any one or a combination selected from the group consisting of C₁-C₄alkyl, C₃-C₅cycloalkyl, halo, hydroxy and C₁₋₃alkoxy; ethyne, hydroxymethyl, amido, N-methylamido and cyano.

In some embodiments, the C₂-C₅heteroaryl of R³ comprises nitrogen and/or sulfur heteroatoms. As described above, C₂-C₅heteroaryl includes thiophenyl, pyridyl, pyrimidinyl, pyrazinyl, imidazolyl, pyrazolyl, thiazolyl and triazolyl. In some embodiments, the C₂-C₅heteroaryl of R³ is any one selected from the group consisting of imidazolyl, pyrazolyl, thiazolyl, pyridyl and triazolyl. For example, the C₂-C₅heteroaryl of R³ may be any one selected from the group consisting of imidazol-5-yl, imidazol-4-yl, imidazol-2-yl, pyrazol-4-yl, 1,3-thiazol-5-yl, pyrid-2-yl, pyrid-3-yl, 1,2,3-triazol-4-yl and 1,2,4-triazol-3-yl.

In some embodiments, the C₂-C₅heteroaryl of R³ comprises nitrogen as the only heteroatom. For example, the C₂-C₅heteroaryl of R³ may be any one selected from the group consisting of imidazolyl, pyrazolyl, pyridyl and triazolyl. In some embodiments, the C₂-C₅heteroaryl of R³ is selected from the group consisting of imidazol-5-yl, imidazol-4-yl, imidazol-2-yl, pyrazol-4-yl, pyrid-2-yl, pyrid-3-yl, 1,2,3-triazol-4-yl and 1,2,4-triazol-3-yl.

In some embodiments, the C₂-C₅heteroaryl of R³ is a C₃heteroaryl. For example, the C₂-C₅heteroaryl may include imidazolyl, pyrazolyl and thiazolyl, such as imidazol-5-yl, imidazol-4-yl, imidazol-2-yl, pyrazol-4-yl and 1,3-thiazol-5-yl. In some embodiments, the C₂-C₅heteroaryl of R³ is any one selected from the group consisting of imidazolyl and pyrazolyl, such as imidazol-5-yl, imidazol-4-yl, imidazol-2-yl and pyrazol-4-yl.

The optional substituents of the C₂-C₅heteroaryl of R³ may be any one or a combination selected from the group consisting of C₁-C₄alkyl, C₃-C₅cycloalkyl, halo, hydroxy and C₁₋₃alkoxy. In some embodiments, the C₁-C₄alkyl is any one or a combination selected from methyl and ethyl, such as methyl. As described above, C₃-C₅cycloalkyl includes cyclopropyl, cyclobutyl and cyclopentyl. In some embodiments, the C₃-C₅cycloalkyl is any one or a combination of cyclobutyl or cyclopropyl.

In some embodiments, the optional substituents of the C₂-C₅heteroaryl of R³ are any one or a combination selected from the group consisting of C₁-C₄alkyl and C₃-C₅cycloalkyl.

In some embodiments, the optional substituents of the C₂-C₅heteroaryl of R³ are one or more methyl.

In some embodiments, R³ is an optionally substituted C₂-C₅heteroaryl.

In some embodiments, R³ is a C₃heteroaryl, optionally substituted one or more times with methyl.

In some embodiments, R³ is any one selected from the group consisting of imidazolyl, 1-methylimidazolyl, 2-methylimidazolyl, 4-methylimidazolyl and pyrazolyl. For example, R³ may be any one selected from the group consisting of imidazol-5-yl, 1-methylimidazol-2-yl, 1-methylimidazol-4-yl, 2-methylimidazol-5-yl, 4-methylimidazol-5-yl and pyrazol-5-yl.

As described above, D is NR⁷ or O, wherein R⁷ is C₁₋₃alkyl. In some embodiments, D is NR⁷, such as NCH₃.

As described above, Re may be H or, alternatively, R⁶ and D may together form a 5 or 6 membered heterocycle optionally substituted one or more times with any one or a combination selected from the group consisting of oxo, halo, C₁₋₃alkyl, C₁₋₃haloalkyl, hydroxy and C₁₋₃alkoxy. In some embodiments, R⁶ and D together form a 5 membered heterocycloalkene or a 6 membered heterocyclodialkene, each optionally substituted one or more times with any one or a combination selected from the group consisting of oxo, halo, C₁₋₃alkyl, C₁₋₃haloalkyl, hydroxy and C₁₋₃alkoxy. In such embodiments, the compound may be of formula (III) or (IV):

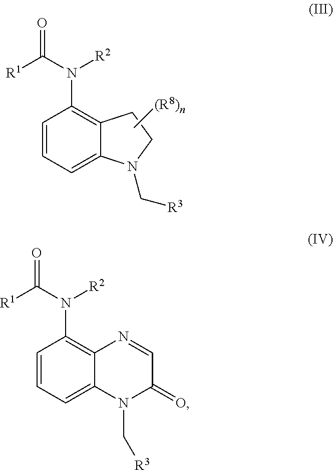

wherein R¹, R² and R³ are as described above and herein;
each R⁸ is independently selected from the group consisting of oxo, halo, C₁₋₃alkyl, C₁₋₃haloalkyl, hydroxy and C₁₋₃alkoxy; and
n is 0 to 4.

In some embodiments, n is 0.

In some embodiments, R⁶ is H.

In particular embodiments, there is provided a compound of formula (V) wherein:

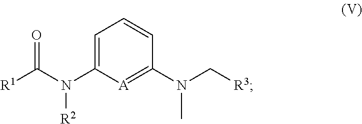

wherein R¹ is any one selected from the group consisting of C₆-C₁₀aryl, C₃-C₅heteroaryl, C₁₀-C₁₆biaryl, C₆-C₁₄biheteroaryl, C₅-C₈cycloalkyl, C₃-C₇heterocycloalkyl, C₃-C₆alkyl, C₆-C₁₀arylC₁-C₃alkyl, C₁₀-C₁₆biarylC₁-C₃alkyl and C₃-C₅cycloalkylC₁-C₃alkyl optionally substituted one or more times with any one or a combination selected from the group consisting of halo, C₁-C₄haloalkyl, C₁-C₄alkyl, C₁-C₄alkoxy, hydroxy and methylsulfonyl;
R² is any one selected from the group consisting of C₁-C₄alkyl, C₃-C₅cycloalkyl, C₃-C₅cycloalkylC₁-C₃alkyl and C₁-C₄alkylether;
A is a carbon or nitrogen atom;
R³ is any one selected from the group consisting of C₃-C₅heteroaryl optionally substituted one or more times with any one or a combination selected from the group consisting of C₁-C₄alkyl C₃-C₅cycloalkyl, ethyne, hydroxymethyl, halo and N-methylacetamido;
R⁴ is any one selected from the group consisting of C₆aryl, C₃-C₇heteroaryl and C₆-C₁₄biheteroaryl optionally substituted one or more times with any one or a combination selected from the group consisting of hydroxy, C₁-C₄alkoxy, C₁-C₄haloalkoxy, halo, C₁-C₄haloalkyl, C₁-C₄alkyl, C₃-C₅cycloalkyl, C₄-C₅heterocycloalkyl, C₁-C₄alkanol, cyano and methane sulfonato; and
R⁵ is C₃-C₅heteroaryl, optionally substituted one or more times with any one or a combination selected from the group consisting of halo and C₁-C₄alkyl.

In particular embodiments, there is provided a compound of formula (V) wherein:
R¹ is any one selected from the group consisting of phenyl, thiophenyl, pyridinyl, cyclohexyl and iso-propyl, optionally substituted one or more times with any one or a combination selected from the group consisting of fluoro, chloro, trifluoromethyl, methyl, ethyl and methoxy;
R² is any one selected from the group consisting of iso-butyl, iso-propyl, ethyl, n-butyl, dimethylpropyl, cyclobutylmethyl and cyclopropylmethyl;
A is a carbon atom; and
R³ is any one selected from the group consisting of imidazol-5-yl, 1-methylimidazol-2-yl, 1-methylimidazol-4-yl, 2-methylimidazol-5-yl, 4-methylimidazol-5-yl and pyrazol-5-yl.

Compounds of formula (II) are also referred to herein as “singleton compounds”. E of such singleton compounds is CR⁴ or N. In some embodiments, E is CR⁴.

R⁴ of such singleton compounds is any one selected from the group consisting of C₆aryl, C₃-C₇heteroaryl, C₆-C₁₄biheteroaryl, H, halo, C₁₋₆alkyl, C₁₋₆haloalkyl, C₁₋₆alkoxy, wherein the C₆aryl, C₃-C₇heteroaryl and C₆-C₁₄biheteroaryl are each optionally substituted one or more times with any one or a combination selected from the group consisting of hydroxy, C₁-C₄alkoxy, halo, C₁-C₄haloalkyl, C₁-C₄alkyl, C₃-C₅cycloalkyl, C₄-C₅heterocycloalkyl, C₁-C₄alkanol, cyano, methane sulfonate and dimethylamido.

As described above, C₆aryl includes phenyl.

C₃-C₇heteroaryl includes pyrazolyl, pyridyl, indazolyl, thiophenyl, pyrimidinyl, pyrazinyl, imidazolyl, thiazolyl, triazolyl, purinyl and pteridinyl. In some embodiments, the C₃-C₇heteroaryl of R⁴ comprises one or more nitrogen atoms. For example, the C₃-C₇heteroaryl may be any one selected from the group consisting of pyrazolyl, pyridyl, indazolyl, pyrimidinyl, pyrazinyl, imidazolyl, thiazolyl, triazolyl, purinyl and pteridinyl.

C₆-C₁₄biheteroaryl includes bipyrazolyl, bipyridyl, biindazolyl, bithiophenyl, bipyrimidinyl, bipyrazinyl, biimidazolyl, bithiazolyl, bitriazolyl, bipurinyl and bipteridinyl. In some embodiments, the C₆-C₁₄biheteroaryl is any one selected from the group consisting of bipyrazolyl, bipyridinyl and biindazolyl.

C₁-C₆alkyl includes methyl, ethyl, n-propyl, iso-propyl, n-butyl, sec-butyl, tert-butyl, n-pentyl, 2-methylbutan-2-yl, 2,2-dimethylpropyl, 3-methylbutyl, pentan-2-yl, pentan-3-yl, 3-methylbutan-2-yl, 2-methylbutyl, n-hexane, hexan-2-yl, hexan-3-yl, 4-methylpentanyl, 4-methylpentan-2-yl, 2-methylpentan-2-yl and 2-methylpentanyl. In some embodiments, C₁₋₆alkyl is C₁₋₃alkyl. In some embodiments, C₁-C₃alkyl is methyl, ethyl or isopropyl.

In some embodiments, the halo substituent is fluoro or chloro, e.g. fluoro.

The C₁₋₆haloalkyl may be a C₁-C₄haloalkyl, such as any one or a combination selected from the group consisting of halomethyl, haloethyl, halo-n-propyl, halo-iso-propyl, halo-n-butyl, halo-sec-butyl, halo-iso-butyl and halo-tert-butyl. In some embodiments, the halo of the C₁-C₆haloalkyl is fluoro. In some embodiments, the C₁-C₆haloalkyl is any one or a combination selected from the group consisting of trifluoromethyl, difluoromethyl and monofluoromethyl, such as trifluoromethyl.

In some embodiments, R⁴ is any one selected from the group consisting of C₆aryl, C₃-C₇heteroaryl and C₆-C₁₄biheteroaryl, each optionally substituted one or more times with any one or a combination selected from the group consisting of hydroxy, C₁-C₄alkoxy, halo, C₁-C₄haloalkyl, C₁-C₄alkyl, C₃-C₅cycloalkyl, C₄-C₅heterocycloalkyl, C₁-C₄alkanol, cyano and methane sulfonate. In some embodiments, R⁴ is any one selected from the group consisting of phenyl and C₃-C₇heteroaryl optionally substituted one or more times with any one or a combination selected from the group consisting of hydroxy, C₁-C₄alkoxy, halo, C₁-C₄haloalkyl, C₁-C₄alkyl, C₃-C₅cycloalkyl, C₄-C₅heterocycloalkyl, C₁-C₄alkanol, cyano and methane sulfonato. In these embodiments, R⁴ is not optionally substituted C₆-C₁₄biheteroaryl.

In some embodiments, R⁴ is any one selected from the group consisting of phenyl, pyrazolyl, pyridinyl and indazolyl optionally substituted one or more times with any one or a combination selected from the group consisting of hydroxy, C₁-C₄alkoxy, C₁-C₄haloalkoxy, halo, C₁-C₄haloalkyl, C₁-C₄alkyl, C₃-C₅cycloalkyl, C₄-C₅heterocycloalkyl, C₁-C₄alkanol, cyano and methane sulfonato.

When R⁴ is pyridine, it may be optionally substituted with methoxy groups at the 2-position. When R⁴ is pyrazolyl, it may be pyrazol-4-yl.

The C₁-C₄alkoxy substituent of R⁴ may be as defined above with respect to R¹, i.e. it may be any one or a combination selected from the group consisting of methoxy, ethoxy, n-propoxy, iso-propoxy, n-butoxy, sec-butoxy, iso-butoxy and tert-butoxy. In some embodiments, the C₁-C₄alkoxy is any one or a combination selected from the group consisting of methoxy and ethoxy.

The C₁-C₄haloalkoxy substituent of R⁴ may be any one or a combination selected from the group consisting of halomethoxy, halomethoxy, halo-n-propoxy, halo-iso-propoxy, halo-n-butoxy, halo-sec-butoxy, halo-iso-butoxy and halo-tert-butoxy. In some embodiments, the halo of the C₁-C₄haloalkoxy substituent is fluoro. In some embodiments, the C₁-C₄haloalkoxy substituent is any one or a combination selected from the group consisting of trifluoromethoxy, difluoromethoxy and monofluoromethoxy, such as trifluoromethoxy.

In some embodiments, the halo substituent is fluoro or chloro, e.g. fluoro.

The C₁-C₄haloalkyl substituent of R⁴ may be as defined above with respect to R¹, i.e. it may be any one or a combination selected from the group consisting of halomethyl, haloethyl, halo-n-propyl, halo-iso-propyl, halo-n-butyl, halo-sec-butyl, halo-iso-butyl and halo-tert-butyl. In some embodiments, the halo of the C₁-C₄haloalkyl is fluoro. In some embodiments, the C₁-C₄haloalkyl is any one or a combination selected from the group consisting of trifluoromethyl, difluoromethyl and monofluoromethyl, such as trifluoromethyl.

In some embodiments, the C₁-C₄alkyl substituent of R⁴ is any one or a combination selected from methyl and ethyl, such as methyl.

As described above, C₃-C₅cycloalkyl includes cyclopropyl, cyclobutyl and cyclopentyl. In some embodiments, the C₃-C₅cycloalkyl is any one or a combination of cyclopropyl or cyclobutyl, such as cyclopropyl.

C₄-C₅heterocycloalkyl includes morpholino, tetrahydropyranyl, piperidinyl, tetrahydrofuranyl, pyrrolidinyl and diazepinyl. In some embodiments, the C₄-C₅heterocycloalkyl substituent is morpholino or tetrahydropyranyl.

C₁-C₄alkanol includes methylene alcohol (—CH₂OH), ethylene alcohol (—CH₂CH₂OH), n-propylene alcohol (—CH₂CH₂CH₂OH), iso-propylene alcohol (—CH(CH₃)CH₂OH), n-butylene alcohol (—CH₂CH₂CH₂CH₂OH), iso-butylene alcohol (—CH₂CH(CH₃)CHOH), sec-butylene alcohol (—CH(CH₃)CH₂CH₂OH) and tert-butylene alcohol (—C(CH₃)₂CH₂OH). In some embodiments, the C₁-C₄alkanol substituent is one or a combination selected from n-propylene alcohol and iso-propylene alcohol.

In some embodiments, the optional substituents of R⁴ are hydroxy, C₁-C₄alkoxy, halo, C₁-C₄haloalkyl, C₁-C₄alkyl, C₃-C₅cycloalkyl, C₄-C₅heterocycloalkyl, C₁-C₄alkanol and cyano. In some embodiments, the optional substituents of R⁴ are hydroxy, C₁-C₂alkoxy, C₁-C₂haloalkoxy, halo, halomethyl, C₁-C₃alkyl, C₁-C₃cycloalkyl, C₄-C₅heterocycloalkyl, C₁-C₂alkanol and cyano. For example, the optional substituents of R⁴ may be hydroxy, fluoro, chloro, methyl, trifluoromethyl, trifluoromethoxy, methoxy, ethoxy, propyl, cyclopropyl, morpholino, tetrahydropyranyl, propanol and cyano.

In some embodiments, the optional substituents of R⁴ are hydroxy, fluoro, chloro, methyl, trifluoromethyl, trifluoromethoxy, methoxy, ethoxy, propyl, morpholino, tetrahydropyranyl and propanol.

In some embodiments, R⁴ is any one selected from the group consisting of phenyl and C₃-C₇heteroaryl optionally substituted one or more times with any one or a combination selected from the group consisting of hydroxy, C₁-C₂alkoxy, halo, halomethyl, C₁-C₃alkyl, C₁-C₃cycloalkyl, C₄-C₅heterocycloalkyl and C₁-C₂alkanol.

In particular embodiments, R⁴ is any one selected from the group consisting of phenyl, pyrazolyl, pyridyl and indazolyl optionally substituted one or more times with any one or a combination selected from the group consisting of hydroxy, fluoro, chloro, methyl, trifluoromethyl, methoxy, ethoxy, propyl, morpholino, tetrahydropyranyl and propanol. In more particular embodiments, the pyridyl of R⁴ is optionally substituted with methoxy groups at the 2-position and the pyrazolyl of R⁴ is pyrazol-4-yl.

As described above, R⁵ is any one selected from the group consisting of C₂-C₅heteroaryl, C₆aryl, C₃₋₅cycloalkyl and C₁₋₄alkyl, wherein the C₂-C₅heteroaryl, C₆aryl and C₃₋₅cycloalkyl are each optionally substituted one or more times with any one or a combination selected from the group consisting of halo and C₁-C₄alkyl. As described above, C₂-C₅heteroaryl includes thiophenyl, pyridyl, pyrimidinyl, pyrazinyl, imidazolyl, pyrazolyl, thiazolyl and triazolyl.

For the avoidance of doubt, the C₃₋₅cycloalkyl may be bridged, for example by a methylene group. In some embodiments, the C₃₋₅cycloalkyl is cyclobutanyl or bicyco[1.1.1]pentane.

The C₁₋₄alkyl includes methyl, ethyl, n-propyl, iso-propyl, n-butyl, sec-butyl, and tert-butyl. In some embodiments, C₁₋₄alkyl is methyl, ethyl, isopropyl or tert-butyl.

In some embodiments, R⁵ is an optionally substituted C₂-C₅heteroaryl.

In some embodiments, R⁵ comprises one or more sulfur and/or nitrogen heteroatoms, i.e. the C₃-C₅heteroaryl of R⁵ comprises one or more sulfur and/or nitrogen heteroatoms. In particular embodiments, the C₃-C₅heteroaryl of R⁵ is any one selected from the group consisting of thiophenyl, pyrazolyl, imidazolyl and thiazolyl. For example, the C₃-C₅heteroaryl of R⁵ may be any one selected from the group consisting of thiophen-2-yl, pyrazol-5-yl, pyrazol-4-yl, imidazol-2-yl and thiazol-5-yl. In some embodiments, the C₃-C₅heteroaryl of R⁵ is not thiazolyl.

In some embodiments, the halo substituent of R⁵ is fluoro and/or chloro.

In some embodiments, the C₁-C₄alkyl substituent of R⁵ is methyl, ethyl and/or propyl. For example, the C₁-C₄alkyl substituent of R⁵ may be methyl.

In some embodiments, R⁵ is any one selected from the group consisting of thiophenyl, pyrazolyl, imidazolyl and thiazolyl, optionally substituted one or more times with any one or a combination selected from the group consisting of fluoro, chloro and methyl.

In particular embodiments, R⁵ is any one selected from the group consisting of thiophen-2-yl, pyrazol-5-yl, pyrazol-4-yl, imidazol-2-yl and thiazol-5-yl, optionally substituted one or more times with any one or a combination selected from the group consisting of fluoro, chloro and methyl. When the C₃-C₅heteroaryl of R⁵ is imidazolyl or pyrazolyl, it may be unsubstituted. For example, when the C₃-C₅heteroaryl of R⁵ is imidazol-2-yl and pyrazol-4-yl, it may be unsubstituted. When the C₃-C₅heteroaryl of R⁵ is thiophenyl, it may be substituted at the 3-position. For example, when the C₃-C₅heteroaryl of R⁵ is thiophen-2-yl, it may be substituted at the 3-position.

In particular embodiments, there is provided a compound of formula (VI):

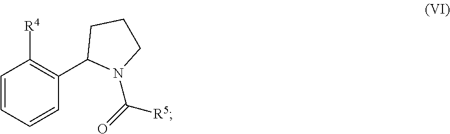

wherein:
R⁴ is any one selected from the group consisting of C₆aryl, C₃-C₇heteroaryl and C₆-C₁₄biheteroaryl optionally substituted one or more times with any one or a combination selected from the group consisting of hydroxy, C₁-C₄alkoxy, C₁-C₄haloalkoxy, halo, C₁-C₄haloalkyl, C₁-C₄alkyl, C₃-C₅cycloalkyl, C₄-C₅heterocycloalkyl, C₁-C₄alkanol, cyano and methane sulfonato; and
   R⁵ is C₃-C₅heteroaryl, optionally substituted one or more times with any one or a combination selected from the group consisting of halo and C₁-C₄alkyl. In particular embodiments, there is provided a compound of formula (VI) wherein:
R⁴ is any one selected from the group consisting of phenyl, pyrazolyl, pyridyl and indazolyl optionally substituted one or more times with any one or a combination selected from the group consisting of hydroxy, fluoro, chloro, methyl, trifluoromethyl, methoxy, ethoxy, propyl, morpholino, tetrahydropyranyl and propanol; and
R⁵ is any one selected from the group consisting of thiophen-2-yl, pyrazol-5-yl, pyrazol-4-yl, thiazol-5-yl and imidazol-2-yl, optionally substituted one or more times with any one or a combination selected from the group consisting of fluoro, chloro and methyl.

In some embodiments, the compound of formula (I) or (II) is selected from the group consisting of ESC1002755, ESC1002842, ESC1002757, ESC1002456, ESC1002799, ESC1002769, ESC1002033, ESC1002575, ESC1002204 and ESC1002597. In some embodiments, the compound of formula (I) or (II) is represented by any one of structures (a to j).

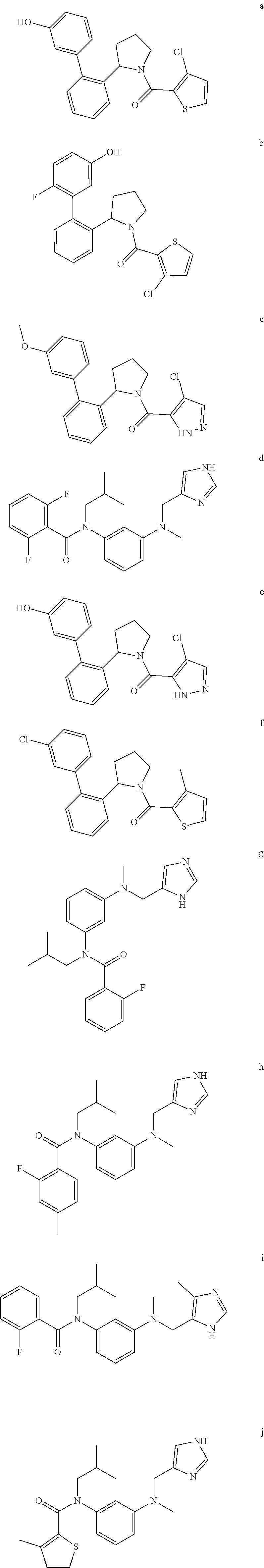

In some cases, the compounds described herein may be isolated or prepared in the form of a pharmaceutically acceptable salt. The term “pharmaceutically acceptable salt” is intended to define salts that may be administered to a patient or used in pharmacy. The pharmaceutically acceptable salt may be prepared by reacting the compound of the invention with a suitable acid, such as hydrogen chloride, hydrogen bromide, hydrogen iodide, sulfuric acid, phosphoric acid, acetic acid, trifluoroacetic acid, propionic acid, glycolic acid, maleic acid, malonic acid, methanesulfonic acid, fumaric acid, succinic acid, tartaric acid, citric acid, benzoic acid and ascorbic acid.

The compounds of the invention (in particular those of formula (II)) may exist in different stereoisomeric forms. All stereoisomeric forms and mixtures thereof, including enantiomers and racemic mixtures, are included within the scope of the invention. Individual stereoisomers of compounds of the invention, i.e. compounds comprising less than 5% 2% or 1% (e.g. less than 1%) of the other stereoisomer, are included. Mixtures of stereoisomers in any proportion, for example a racemic mixture comprising substantially equal amounts of two enantiomers are also included within the invention.

Compounds of formula (II) comprise at least one chiral centre. The carbon atom at position 2 of the pyrrolidine centre of compounds of formula (II) is chiral. The inventors have found that the S (sinister/left) enantiomers of compounds of formula (II) are consistently more active and thus more effective as ABAD inhibitors than the R-(rectus/right) enantiomers. Thus, in some embodiments, when the compound is of formula (II), it comprises about 60% to about 100%, about 70% to about 100%, about 80% to about 100%, or about 90% to about 100% of the S enantiomer. For example, the compound may comprise about 90% to about 100% of the S enantiomer. In some embodiments, when the compound is of formula (II), it comprises substantially just the S enantiomer, i.e. it comprises >95%, >98% or >99% S enantiomer.

R- and S-enantiomers are distinguishable by the direction of priority of the substituents attached to the chiral centre. Priority is based on the atomic number (proton number) of the first atom of the substituent. For example, if the carbon atom at position 2 of the pyrrolidine centre of a compound of formula (II) is bound to a substituted phenyl, the nitrogen atom of the pyrrolidine, a proton and the carbon atom at position 3 of the pyrrolidine, the priority of the substituents (from lowest to highest) is in the order of proton<carbon at position 3 of pyrrolidine<substituted phenyl<nitrogen atom of pyrrolidine. In the case of carbon at position 3 of pyrrolidine and substituted phenyl, the first atom of the substituent is carbon. In order to distinguish priority of these two substituents, the second atoms of the substituents are taken into account. For the carbon at position 3 of pyrrolidine, two of the second atoms are hydrogen and one is carbon, whereas for substituted phenyl the second atoms are all carbon. Since carbon has a higher atomic number than hydrogen, it takes priority. Hence, substituted phenyl has a greater priority than the carbon at position 3 of pyrrolidine. To distinguish whether the chiral centre at position 2 of pyrrolidine is R or S, the chiral centre is oriented so that the lowest-priority of the four substituents (e.g. proton) is pointed away from the plane of view. If the priority of the remaining three substituents decreases in a clockwise direction, the enantiomer is R, and if the priority decreases in a counterclockwise direction, the enantiomer is S.

Also included are solvates and isotopically-labelled compounds of formula (I) or (II). Isotopically-labelled compounds are identical to those described herein, with the exception that one or more atoms are replaced by an atom having an atomic mass or mass number different from the atomic mass or mass number predominantly found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively.

Also provided herein is a composition comprising a compound of the first aspect and one or more pharmaceutically acceptable excipients. For the avoidance of doubt, the embodiments described herein in relation to the first aspect of the invention apply mutatis mutandis to the composition of the second aspect. For example, the composition of the second aspect may comprise a compound of formula (V) or (VI), wherein:
R¹ is any one selected from the group consisting of phenyl, thiophenyl, pyridinyl, cyclohexyl and iso-propyl, optionally substituted one or more times with any one or a combination selected from the group consisting of fluoro, chloro, trifluoromethyl, methyl, ethyl and methoxy;
R² is any one selected from the group consisting of iso-butyl, iso-propyl, ethyl, n-butyl, dimethylpropyl, cyclobutylmethyl and cyclopropylmethyl;
A is a carbon atom;
R³ is any one selected from the group consisting of imidazol-5-yl, 1-methylimidazol-2-yl, 1-methylimidazol-4-yl, 2-methylimidazol-5-yl, 4-methylimidazol-5-yl and pyrazol-5-yl;
R⁴ is any one selected from the group consisting of phenyl, pyrazolyl, pyridyl and indazolyl optionally substituted one or more times with any one or a combination selected from the group consisting of hydroxy, fluoro, chloro, methyl, trifluoromethyl, methoxy, ethoxy, propyl, morpholino, tetrahydropyranyl and propanol; and
R⁵ is any one selected from the group consisting of thiophen-2-yl, pyrazol-5-yl, pyrazol-4-yl, thiazol-5-yl and imidazol-2-yl, optionally substituted one or more times with any one or a combination selected from the group consisting of fluoro, chloro and methyl.

When the compound is of formula (II), it may comprise about 60% to about 100%, about 70% to about 100%, about 80% to about 100%, or about 90% to about 100% of the S enantiomer.

The composition of the second aspect comprises one or more pharmaceutically acceptable excipients. An extensive overview of pharmaceutically acceptable excipients is described in the Handbook of Pharmaceutical Excipients, 6^{th} Edition; Editors R. C. Rowe, P. J. Sheskey and M. E. Quinn, The Pharmaceutical Press, London, American Pharmacists Association, Washington, 2009. Any suitable pharmaceutically acceptable excipient described within this document is within the scope of the invention.

The pharmaceutically acceptable excipient may be included within the composition for the purpose of long-term stabilization of the compound, bulking up solid formulations (often referred to as “bulking agents”, “fillers”, or “diluents”), or to enhance activity of the compound, such as by facilitating its absorption within the body, reducing its viscosity, or enhancing its solubility. The excipient may also enhance in vitro stability of the compound, such as prevention of denaturation or aggregation. Alternatively, the excipient may be used for identification purposes, or to make the compound more appealing to the patient, for example by improving its taste, smell and/or appearance. Typically, the excipient makes up the bulk of the composition.

Excipients include diluents or fillers, binders, disintegrants, lubricants, colouring agents and preservatives. Diluents or fillers are inert ingredients that may affect the chemical and physical properties of the final composition. If the dosage of the compound of the invention is small then more diluents will be required to produce a composition suitable for practical use. If the dosage of the compound of the invention is high then fewer diluents will be required.

Binders add cohesiveness to powders in order to form granules, which may form a tablet. The binder must also allow the tablet to disintegrate upon ingestion so that the compound of the invention dissolves. Disintegration of the composition after administration may be facilitated through the use of a disintegrant.

In a third aspect, there is provided a compound of the first aspect or composition of the second aspect for use as an ABAD inhibitor. As described above, the inventors have found that the compounds disclosed herein are surprisingly effective ABAD inhibitors. The main function of ABAD, is to produce energy for the brain. In AD, a build-up of amyloid within mitochondria, allows amyloid to attach to ABAD, altering its function and ultimately impairing the memory capabilities of the brain. Direct modulation of ABAD activity is reported to be effective in treating AD (Lim et al., PLoS One, 2011, 6 (12), e28887). In addition, ABAD has been implicated in various forms of cancer, such as prostate, breast and bone cancer and notably in cases of prostate cancer deemed castration resistant prostate cancer (CRPC).

Accordingly, viewed from a fourth aspect, there is provided a compound of the first aspect or a composition of the second aspect for use as a medicament and in a fifth aspect, there is provided a compound of the first aspect or a composition of the second aspect for use in a method of treatment or prophylaxis of dementia or cancer. In some embodiments, the compound or composition described herein is for use in a method of treatment or prophylaxis of dementia, prostate cancer, bone cancer or breast cancer. In some embodiments, the use is in a method of treatment or prophylaxis of AD, prostate cancer, bone cancer or breast cancer.

The compounds or compositions described herein may be suitable for enteral (including oral, such as buccal or sublingual, and nasal administration) or parenteral (including subcutaneous, intravenous and intramuscular) administration. In some embodiments, the compounds or compositions described herein are suitable for parenteral administration.

The compounds or compositions described herein may be compressed into solid dosage units, such as tablets, or be processed into capsules. The compounds or compositions may be prepared in the form of a solution, suspension or emulsion for injection. Alternatively, the compounds or compositions may be administered as a spray, including a nasal or buccal spray. In some embodiments, the compounds or compositions are prepared in the form of a solution, suspension or emulsion for injection.

Viewed from a sixth aspect, there is provided a method of inhibiting ABAD activity in a subject, the method comprising administering an effective amount of a compound of the first aspect or a composition of the second aspect.

Viewed from a seventh aspect, there is provided a method of treatment comprising administering an effective amount of a compound of the first aspect or a composition of the second aspect to a subject.

Viewed from an eighth aspect, there is provided a method for the treatment or prophylaxis of dementia or cancer, the method comprising administering an effective amount of a compound of the first aspect or a composition of the second aspect to a subject.

An effective amount of the compound or composition may be administered to a subject enterally or parenterally. The compound or composition may be administered parenterally, sometimes by direct injection, which is typically intramuscular, subcutaneous or intraveneous, or enterally via a tablet, capsule or buccal spray.

The subject may be any animal, such as a human, a zoo or farm animal or a domestic animal (a pet), i.e. the compound or composition may be for veterinary use. The subject is typically a human, and may be suffering from or liable to suffer from AD or cancer.

The skilled person is aware that an effective amount is likely to vary with the particular compound or composition of the invention, the subject and the administration procedure used. The skilled person is able to identify the effective amount of the compounds and compositions of the invention via routine work and experimentation.

In some embodiments, the method is for the treatment or prophylaxis of dementia, prostate cancer, bone cancer or breast cancer. In some embodiments, the method is for the treatment or prophylaxis of AD, prostate cancer, bone cancer or breast cancer.

For the avoidance of doubt, the embodiments described herein in relation to the first aspect and second aspects apply mutatis mutandis to the compounds and compositions of the fourth to eighth aspects.

Any discussion herein of documents, acts, materials, devices, articles or the like is not to be taken as an admission that any or all of these matters form part of the prior art base or were common general knowledge in the field relevant to the present disclosure as it existed before the priority date of each claim of this application.

It will be appreciated by those skilled in the art that numerous variations and/or modifications may be made to the invention as described herein without departing from the scope of the invention as described. The present embodiments are therefore to be considered for descriptive purposes and are not restrictive, and are not limited to the extent of that described in the embodiment. The person skilled in the art is to understand that the present embodiments may be read alone, or in combination, and may be combined with any one or a combination of the features described herein.

The subject-matter of each patent and non-patent literature reference cited herein is hereby incorporated by reference in its entirety.

The invention may be further understood with reference to the following clauses:
1. A compound of formula (I) or (II):

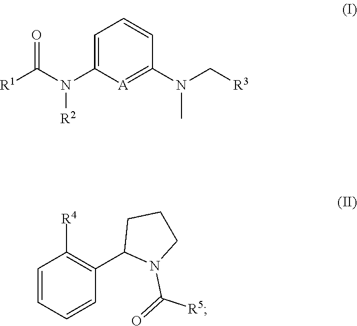

wherein R¹ is any one selected from the group consisting of C₆-C₁₀aryl, C₃-C₅heteroaryl, C₁₀-C₁₆biaryl, C₆-C₁₄biheteroaryl, C₅-C₈cycloalkyl, C₃-C₇heterocycloalkyl, C₃-C₆alkyl, C₆-C₁₀arylC₁-C₃alkyl, C₁₀-C₁₆biarylC₁-C₃alkyl and C₃-C₈cycloalkylC₁-C₃alkyl optionally substituted one or more times with any one or a combination selected from the group consisting of halo, C₁-C₄haloalkyl, C₁-C₄alkyl, C₁-C₄alkoxy, hydroxy and methylsulfonyl;
   R² is any one selected from the group consisting of C₁-C₄alkyl, C₃-C₅cycloalkyl, C₃-C₅cycloalkylC₁-C₃alkyl and C₁-C₄alkylether;
   A is a carbon or nitrogen atom;
   R³ is any one selected from the group consisting of C₃-C₅heteroaryl optionally substituted one or more times with any one or a combination selected from the group consisting of C₁-C₄alkyl C₃-C₅cycloalkyl, ethyne, hydroxymethyl, halo and N-methylacetamido;
   R⁴ is any one selected from the group consisting of C₆aryl, C₃-C₇heteroaryl and C₆-C₁₄biheteroaryl optionally substituted one or more times with any one or a combination selected from the group consisting of hydroxy, C₁-C₄alkoxy, C₁-C₄haloalkoxy, halo, C₁-C₄haloalkyl, C₁-C₄alkyl, C₃-C₅cycloalkyl, C₄-C₅heterocycloalkyl, C₁-C₄alkanol, cyano and methane sulfonato; and
   R⁵ is C₃-C₅heteroaryl, optionally substituted one or more times with any one or a combination selected from the group consisting of halo and C₁-C₄alkyl.
2. The compound of clause 1, wherein R¹ is any one selected from the group consisting of C₆-C₁₀aryl, C₃-C₅heteroaryl, C₁₀-C₁₆biaryl, C₅-C₈cycloalkyl, C₃-C₆alkyl and
   C₆-C10arylC₁-C₃alkyl, optionally substituted one or more times with any one or a combination selected from the group consisting of halo, C₁-C₄haloalkyl, C₁-C₄alkyl, C₁-C₄alkoxy, hydroxy and methylsulfonyl.
3. The compound of clause 1, wherein R¹ is any one selected from the group consisting of C₆-C₁₀aryl, C₃-C₅heteroaryl, C₅-C₈cycloalkyl and C₃-C₆alkyl, optionally substituted one or more times with any one or a combination selected from the group consisting of halo, C₁-C₄haloalkyl, C₁-C₄alkyl, C₁-C₄alkoxy and hydroxy.
4. The compound of clause 1, wherein R¹ is any one selected from the group consisting of phenyl, thiophenyl, pyridinyl, cyclohexyl and iso-propyl, optionally substituted one or more times with any one or a combination selected from the group consisting of fluoro, chloro, trifluoromethyl, methyl, ethyl and methoxy.
5. The compound of any one of clauses 1 to 4, wherein R² is any one selected from the group consisting of C₁-C₄alkyl and C₃-C₅cycloalkylC₁-C₃alkyl.
6. The compound of any one of clauses 1 to 4, wherein R² is any one selected from the group consisting of iso-butyl, iso-propyl, ethyl, n-butyl, dimethylpropyl, cyclobutylmethyl and cyclopropylmethyl.
7. The compound of any one of clauses 1 to 6, wherein A is a carbon atom.
8. The compound of any one of clauses 1 to 7, wherein R³ comprises nitrogen and/or sulfur as heteroatoms.
9. The compound of any one of clauses 1 to 7, wherein R³ comprises nitrogen as the only heteroatom.
10. The compound of any one of clauses 1 to 9, wherein R³ is a C₃heteroaryl, optionally substituted one or more times with methyl.
11. The compound of any one of clauses 1 to 7, wherein R³ is any one selected from the group consisting of imidazolyl, 1-methylimidazolyl, 2-methylimidazolyl, 4-methylimidazolyl and pyrazolyl.
12. The compound of any one of clauses 1 to 7, wherein R³ is any one selected from the group consisting of imidazol-5-yl, 1-methylimidazol-2-yl, 1-methylimidazol-4-yl, 2-methylimidazol-5-yl, 4-methylimidazol-5-yl and pyrazol-5-yl.
13. The compound of any one of clauses 1 to 12, wherein R⁴ is any one selected from the group consisting of phenyl and C₃-C₇heteroaryl optionally substituted one or more times with any one or a combination selected from the group consisting of hydroxy, C₁-C₂alkoxy, C₁-C₂haloalkoxy, halo, halomethyl, C₁-C₃alkyl, C₁-C₃cycloalkyl, C₄-C₅heterocycloalkyl, C₁-C₂alkanol and cyano.
14. The compound of any one of clauses 1 to 12, wherein R⁴ is any one selected from the group consisting of phenyl, pyrazolyl, pyridinyl and indazolyl optionally substituted one or more times with any one or a combination selected from the group consisting of hydroxy, fluoro, chloro, methyl, trifluoromethyl, trifluoromethoxy, methoxy, ethoxy, propyl, morpholino, tetrahydropyranyl, propanol and cyano.
15. The compound of any one of clauses 1 to 14, wherein R⁵ comprises one or more sulfur and/or nitrogen heteroatoms.
16. The compound of any one of clauses 1 to 14, wherein R⁵ is any one selected from the group consisting of thiophenyl, pyrazolyl, thiazolyl and imidazolyl, optionally substituted one or more times with any one or a combination selected from the group consisting of fluoro, chloro and methyl.
17. The compound of any one of clauses 1 to 14, wherein R⁵ is any one selected from the group consisting of thiophen-2-yl, pyrazol-5-yl, pyrazol-4-yl, thiazol-5-yl and imidazol-2-yl, optionally substituted one or more times with any one or a combination selected from the group consisting of fluoro, chloro and methyl.
18. A composition comprising the compound of any one of clauses 1 to 17 and one or more pharmaceutically acceptable excipients.
19. A compound of any one of clauses 1 to 17 or a composition of clause 18 for use as an ABAD inhibitor.
20. A compound of any one of clauses 1 to 17 or a composition of clause 18 for use as a medicament.
21. A compound of any one of clauses 1 to 17 or a composition of clause 18 for use in a method of treatment or prophylaxis of cancer.
22. The compound or composition for use of clause 21, wherein the use is in a method of treatment of cancer.
23. The compound or composition for use of clause 21, wherein the use is in a method of prophylaxis of cancer.
24. A compound of any one of clauses 1 to 17 or a composition of clause 18 for use in a method of treatment or prophylaxis of dementia.
25. The compound or composition for use of clause 24, wherein the use is in a method of treatment of dementia.
26. The compound or composition for use of clause 24, wherein the use is in a method of prophylaxis of dementia.
27. A method of inhibiting ABAD activity in a subject, the method comprising administering an effective amount of a compound of any one of clauses 1 to 17 or a composition of clause 18.
28. A method of treatment comprising administering an effective amount of a compound of any one of clauses 1 to 17 or a composition of clause 18 to a subject.
29. A method for the treatment or prophylaxis of cancer, the method comprising administering an effective amount of a compound of any one of clauses 1 to 17 or a composition of clause 18 to a subject.
30. The method of clause 29, wherein the method is for the treatment of cancer.
31. The method of clause 29, wherein the method is for the prophylaxis of cancer.
32. A method for the treatment or prophylaxis of dementia, the method comprising administering an effective amount of a compound of any one of clauses 1 to 17 or a composition of clause 18 to a subject.
33. The method of clause 32, wherein the method is for the treatment of dementia.
34. The method of clause 32, wherein the method is for the prophylaxis of dementia.

The invention may be further understood with reference to the examples that follow.

### EXAMPLES

### Materials and Methods

All aqueous solutions were prepared with deionized water (Millipore, UK) and all chemicals purchased from Sigma Aldrich, UK unless stated otherwise.

### Protein Purification for Enzyme Activity Assays

See Aitken, L et al. 2016, ChemBioChem, vol. 17, no. 11, pp. 1029-1037

Cell pellets of E. coli BL21-CodonPlus cells containing Histev-17β-HSD10 protein were re-suspended for 30 min, 4° C., in lysis buffer (20 mM NaH₂PO₄, 30 mM imidazole, 500 mM NaCl, 10% (v/v) glycerol, pH 7.5) with the addition of complete ethylenediaminetetraacetic acid (EDTA)-free protease inhibitor tablets (Roche), lysozyme (1 mg/mL), deoxyribonuclease (DNase) (20 μg/mL) and Triton X-100 (0.1% (v/v)). Cells were lysed by passage through a cell disruptor at 30 kPSI (Constant Systems Ltd) and the lysate was cleared by centrifugation (Sorvall Evolution RC, rotor S5-34 55-34 angle, 20500 rpm, 30 min, 4° C.). Cleared lysate was filtered (0.45 μm membrane; Whatman) then applied to a Ni-nitrilotriacetic acid (NTA) (GE Healthcare) column pre-washed with lysis buffer and protein eluted with 300 mM imidazole buffer (20 mM NaH₂PO₄, 300 mM imidazole, 500 mM NaCl, 10% (v/v) glycerol, pH 7.5). Tobacco etch virus (TEV) protease was added to the protein at a mass-to-mass ratio of 1:10, to cleave the histidine tag and the protein was then dialysed into 20 mM Tris-HCl, 30 mM imidazole, 500 mM NaCl, 10% (v/v) glycerol, pH 7.5 containing EDTA (1 mM) and dithiothreitol (DTT) (1 mM) to aid solubility. Protein digestion and dialysis was carried out at 4° C. for 16 h.

Complete digestion was firstly checked by sodium dodecyl sulfate-polyacrylamide gel electrophoresis (SDS-PAGE), then fully digested protein was passed over a second Ni-column and the flow-through, containing 17β-HSD10 protein, was concentrated using a Vivaspin column (10 kDa MWCO, GE Healthcare) to ˜7 mL before final purification using gel filtration to remove imidazole (Hi-Load 16/60 Superdex 75 prep grade column, GE Healthcare, flow rate 1.5 mL/min). Protein was eluted in gel filtration buffer (10 mM trisaminomethane (Tris-HCl), 150 mM NaCl, 10% glycerol, pH 7.5) and concentrated (Vivaspin column (10 kDa molecular weight cut off (MWCO), GE Healthcare)) to 10 mg/mL. Aliquots (10 μL) were taken and flash frozen in liquid nitrogen before final storage at −80° C.

### Compound Screening

(See Aitken, L et al., 2017, SLAS Discovery, vol. 22, no. 6, pp. 676-685.)

The assay buffer used contains: 10 mM Tris. HCl (pH 7.4), 150 mM NaCl, 1 mM DTT, 0.005% Tween (polysorbate 20) and 0.01% bovine serum albumin (BSA). As indicated for some experiments the assay buffer was supplemented with 100 units/ml bovine liver catalase. All experiments were conducted in clear bottom 384-well or 1536-well low volume microplates (Corning) with a final assay volume of 20 μL and the reaction progress was monitored by oxidation of NADH as determined by reduction in absorbance at 340 nm read on the EnVision plate reader (PerkinElmer).

For primary screening an endpoint assay format was adopted in which an Echo acoustic liquid dispenser (Labcyte) was used to transfer 20 nL of either reference standard, dimethylsulfoxide (DMSO) control (0.5% final assay concentration (FAC)) or test compounds (10 μM FAC) to the assay plate. 10 μL of 17β-HSD10 enzyme in assay buffer (2.5 nM, FAC) was then added to the plates using the Preddator liquid handling robot (Redd and Whyte) and plates were incubated at room temperature for 15 minutes. 10 μL of substrate mixture (100 μM acetoacetyl-CoA and 100 μM NADH, FAC) was added with the Preddator. To start the reaction and the plates were left to incubate for 35 minutes at room temperature before the absorbance at 340 nm was read on the EnVision plate reader (PerkinElmer). For kinetic assay format and dose response follow up experiments the same protocol was adopted but the absorbance at 340 nm was monitored constantly for 45 minutes from immediately after the reaction was started.

### Orthogonal Assays

Previous studies during assay development (Aitken et al. 2017, supra) have shown that the primary screen may be susceptible to identifying redox cycling compounds and small molecule aggregating compounds as false positives. Thus, TritonX-100 aggregation assays were run to screen for compounds which could be causing inhibition via aggregation. The primary screen was repeated with the addition of TritonX-100 at a final assay concentration of 0.01% (v/v). To identify potential redox cycling compounds, the primary screen was repeated with the addition of resazurin at a final assay concentration of 50 μM and no addition of protein. The known redox cycler, 9,10-phenanthrenequinone (9, 10 PQ, 40 nM) was used as a positive control and DMSO (1% v/v) acted as the negative control. Compounds were screened at a final assay concentration of 100 M, in triplicate, in 96-well black flat-clear bottom plates. After the protein-substrate mixes with compounds were dispensed, the plate was left to incubate in the dark for 30 minutes. The change in fluorescence was then monitored over a 25-minute period (excitation=560 nm, emission=590 nm).

### Acetoacetyl-CoA Enzyme Kinetics (Aitken et al. 2017, Supra)

To determine the kinetics for acetoacetyl-CoA and the effect of 17β-HSD10 concentration upon reaction rate, a matrix titration experiment was set up. Previously determined conditions were used with a fixed concentration of 700 μM NADH and a starting concentration of up to 200 μM acetoacetyl-CoA. Doubling dilutions of acetoacetyl-CoA were then coupled with doubling dilutions of 17β-HSD10, which started with a maximum concentration of 40 nM. Data collected from the experiment was analysed using a standard template, from which reaction progress curves were analysed to calculate initial velocities and obtain Km values using the Michaelis-Menten equation (XLFit, ID Business Solutions).

### NADH Enzyme Kinetics (Aitken et al. 2017, Supra)

To determine the kinetics for NADH and the effect of 17β-HSD10 concentration upon reaction rate a matrix titration experiment was set up. Previously determined conditions (Aitken et al. 2017, supra) were used, 120 μM acetoacetate and a starting concentration of up to 1000 μM NADH. Doubling dilutions of NADH were then coupled with doubling dilutions of 17β-HSD10, which started with a maximum concentration of 40 nM. Data were analysed as described previously (Aitken et al. 2017, supra).

### Physiochemical Predictions (Aitken et al. 2017, Supra)

Published values were obtained (where available) from DrugBank (http://www.drugbank.com) and where there were no published values, predictions were made using ChemAxon Marvin suite (http://www.chemaxon.com).

### Thermal Shift Analysis

Solutions containing compound at two concentrations (25 μM and 100 μM) added to assay buffer (50 mM Hepes, 0.5 M NaCl, pH 8.2, SYPRO orange 20 X, ±1 mM NADH) were plated in triplicate (25 μl per well) in MicroAmp 96-well plates (Thermo Fisher: 4346907). The plates were then sealed with optical grade sealing film (Thermo Fisher: 4311971) and loaded into a ViiA 7 RT-PCR machine ((Thermo Fisher: 4453534) (ramp speed 0.015° C./sec, 25-95° C.)). Data was exported to GraphPad PRISM and Tₘ values were calculated using the Boltzmann sigmoidal curve fit equation.

### Lactate Dehydrogenase (LDH) Cytotoxicity Assay

Cell cytotoxicity was assessed via the measurement of lactate dehydrogenase leakage into the culture medium using a commercially available kit from Pierce (Thermo Scientific cat no. 88953). This was carried out in accordance with the kit guidelines, with the activity of Lactate Dehydrogenase (LDH) being calculated from the change in absorbance at 340 nm as NADH is reduced. HEK293 cells overexpressing mts17β-HSD10 were cultured in phenol-red free media (10% fetal bovine serum (FBS), 1 mM sodium pyruvate, 100 units penicillin, 0.1 mg/mL streptomycin and 2 mM L-glutamine) and seeded at a density of 10,000 cells per well (100 μL, 96 well plates). Cells were then treated with the compound of interest at 2 concentrations (25 μM and 100 μM in DMSO) in triplicate. Treated cells were incubated at 37° C. and CO₂ (5%) for 24 hours before the LDH assay was performed as per the manufacturer's instructions. Spontaneous control (water) and maximum control (lysis buffer) used in accordance with the kit guide. Absorbance was measured at 490 nm and 680 nm using a SpectraMaxM2e spectrophotometer. The measured LDH activity was used to calculate % cytotoxicity using the following equation:

$$\% cytotoxicity = \frac{( compound treated LDH Activity - Spontaneous LDH Activity )}{( Maximum LDH Activity - Spontaneous LDH Activity )} x 100$$

### Alamar Blue Cell Viability Assay

Cell viability was assessed via fluorescence change using the commercially available Alamar Blue from ThermoFisher. HEK293 cells overexpressing mts17β-HSD10 were cultured in phenol-red free media (10% FBS, 1 mM sodium pyruvate, 100 units penicillin, 0.1 mg/mL streptomycin and 2 mM L-glutamine) and seeded at a density of 10,000 cells per well (100 μL, 96 well plates). Cells were then treated with the compound of interest at 2 concentrations (25 μM and 100 μM in DMSO) in triplicate. Treated cells were then incubated at 37° C. and CO₂ (5%) for 24 hours before the assay was performed as per the manufacturer's instructions. Fluorescence was measured at 530 nm excitation and 590 nm emission using a SpectraMaxM2e spectrophotometer.

### CHANA Assay—In Vitro Dose Response and IC₅₀ Determination

HEK293 mts17β-HSD10 cells were seeded at a density of 10,000 cells per well (100 μL, 96 well black plates) in phenol-red free media (10% FBS, 1 mM sodium pyruvate, 100 units penicillin, 0.1 mg/mL streptomycin and 2 mM L-glutamine). After 24 hours the media was removed from the cells and replaced with fresh media containing varying concentrations of compound (100 μM-0.098 μM). The fluorogenic probe (-)-CHANA was then added to each well to give a final assay concentration of 20 μM. Fluorescence was immediately measured using a FLUOstar Optima microplate reader (excitation=380 nm, emission=520 nm, orbital averaging=3 mm) and the initial reaction monitored for 3-4 hours. IC₅₀ was calculated from the control-subtracted triplicates using non-linear regression (four parameters) of GraphPad Prism 5 software. Final IC₅₀ and SEM value was obtained as a mean of at least 3 independent measurements.

### Chemical Preparation and Synthesis

Standard experimental procedures were followed for synthesis; chemicals and solvents were from commonly used suppliers and were used without further purification. Silica gel 60 F254 analytical thin layer chromatography (TLC) plates were from Merck (Darmstadt, Germany) and visualized under UV light and/or with potassium permanganate stain. Chromatographic purifications were performed using Merck Geduran 60 silica (40-63 μm) or prepacked SNAP columns using a Biotage SP1 Purification system (Uppsala, Sweden). Microwave assisted reactions were performed using a Biotage Initiator™ microwave synthesizer in sealed vials. Deuterated solvents were obtained from Cambridge Isotopes, Sigma-Aldrich, Goss Scientific Instruments Ltd. and Apollo Scientific Ltd. All 1H and 13C NMR spectra were recorded using a Bruker Avance 400 MHz spectrometer. All chemical shifts are given in ppm relative to the solvent peak and coupling constants (J) are reported in Hz. Low Resolution (LR) mass spectrometry data (m/z) were obtained from an Agilent 6140 series Quadrupole Mass Spectrometer with a multimode source attached to an Agilent 1200 series HPLC.

### Analytical Methods

### Liquid-Chromatography-Mass Spectrometry (LC-MS)

### Analytical Method A

LC-MS was performed using an Agilent 6140 Series Quadrupole Mass Spectrometer with a multimode source. Analysis was performed using either a Phenomenex Luna® C18 (2)-HST column (2.5 μm, 50×2.0 mm) or a Waters X-Select® CSH™ C18 column (2.5 μm, 50×2.1 mm). Mobile phase A contained 0.1% formic acid in 18 MΩ water and mobile phase B contained 0.1% formic acid in HPLC grade acetonitrile. A flow rate of 1.00 mL min-1 was used over a 3.75 min gradient starting with 99% mobile phase A gradually increasing to 100% mobile phase B. The samples were monitored at 254 nm.

### Analytical Method B

LC-MS was performed using an Agilent 6140 Series Quadrupole Mass Spectrometer with a multimode source. Analysis was performed using either a Phenomenex Luna® C18 (2)-HST column (2.5 μm, 50×2.0 mm) or a Waters X-Select® CSH™ C18 column (2.5 μm, 50×2.1 mm). Mobile phase A contained 0.1% formic acid in 18 MΩ water and mobile phase B contained 0.1% formic acid in HPLC grade acetonitrile. A flow rate of 1.00 mL min-1 was used over a 5.5 min gradient starting with 99% mobile phase A gradually increasing to 100% mobile phase B. The samples were monitored at 254 nm.

### Analytical Method C

LC-MS was performed using an Agilent 6140 Series Quadrupole Mass Spectrometer with a multimode source. Analysis was performed using either a Phenomenex Luna® C18 (2)-HST column (2.5 μm, 50×2.0 mm) or a Waters X-Select® CSH™ C18 column (2.5 μm, 50×2.1 mm). Mobile phase A contained 0.01M NH₄OH in 18 MΩ water and mobile phase B contained 0.01M NH₄OH in HPLC grade MeOH. A flow rate of 1.00 mL min-1 was used over a 3.75 min gradient starting with 99% mobile phase A gradually increasing to 100% mobile phase B. The samples were monitored at 254 nm.

### Preparative High Performance Liquid Chromatography (HPLC) Method

Preparative HPLC was carried out on a Waters HPLC comprising of a Waters 2767 Sample Manager, Waters 2545 Binary Gradient Module, Waters Systems Fluidics Organiser, Waters 515 ACD pump, Waters 2998 Photodiode Array Detector, using a Waters XBridge Prep OBD C18, 5 μm, 19 mm×50 mm i.d. column and a flow rate of 20 mL/minute. The general method that may be used to purify compounds are: acidic reverse phase HPLC (water/acetonitrile/0.1% trifluoroacetic acid) using a standard gradient of 5% acetonitrile/95% water to 100% acetonitrile or basic reverse phase HPLC (water/acetonitrile/0.01 M ammonia solution) using a standard gradient of 10% acetonitrile/90% water to 100% acetonitrile. UV detection e.g. 254 nM is used for the collection of fractions from HPLC. This description gives general methods and variations in types of equipment, columns, mobile phase, detection wavelength, solvent gradient and run time may also be used to purify compounds.

### Synthesis of Selected Compounds

The chemical synthesis of an exemplified compound of formula (I) is outlined in Schemes 1 and 2, and the synthesis of an exemplified compound of formula (II) is outlined in Scheme 3.

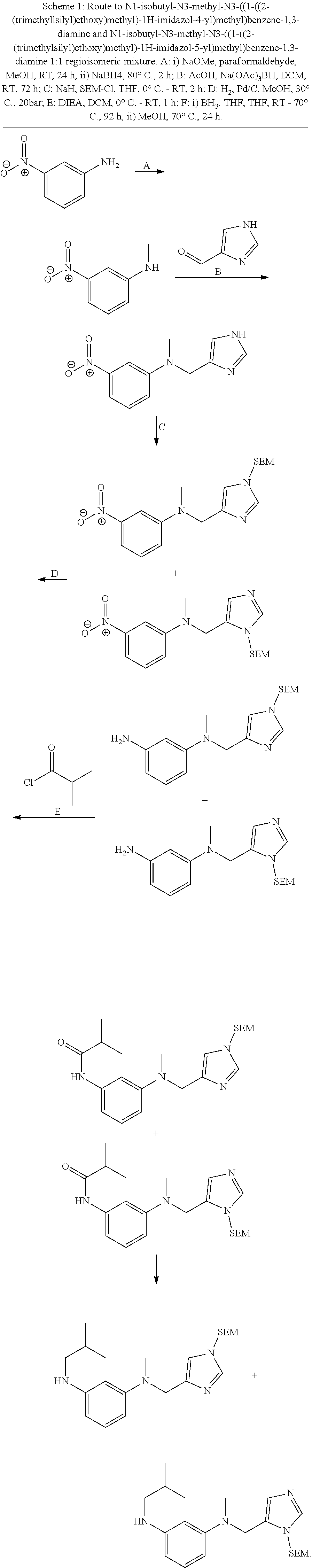

### Synthesis of N-Methyl-3-Nitroaniline

3-nitroaniline (5 g, 36.2 mmol) was added to a suspension of NaOMe (95%, 10.29 g, 181 mmol) in MeOH (30 mL) and the resulting suspension stirred and then added to a stirring suspension of paraformaldehyde (1.52 g, 50.68 mmol) in methanol (40 mL). The reaction was stirred at room temperature for 18 hours then NaBH₄ (1.37 g, 36.2 mmol) was added and the reaction heated to reflux for 2 hours. The reaction mixture was allowed to cool to room temperature then concentrated in vacuo. The mixture was partitioned between water (100 ml) and EtOAc (150 ml). The aqueous layer was separated and extracted with additional EtOAc (100 ml). Organics were combined, washed with brine, dried over sodium sulphate, filtered and solvent was evaporated in vacuo. The crude product was purified by flash column chromatography (silica column, 0% to 100% DCM in n-heptane gradient) followed by concentration of the appropriate fractions in vacuo to afford N-methyl-3-nitro-aniline as an orange solid (4.84 g, 88%). ¹H NMR (400 MHz, CDCl₃) δ 7.54 (dd, J=1.51, 8.03 Hz, 1H), 7.41 (t, J=2.26 Hz, 1H), 7.26-7.33 (m, 1H), 6.89 (dd, J=2.01, 8.03 Hz, 1H), 4.10 (br. s., 1H), 2.93 (br. s., 3H).

### Synthesis of N-((1H-imidazol-4-yl)methyl)-N-methyl-3-nitroaniline

N-methyl-3-nitro-aniline (4 g, 26.29 mmol) and 1H-imidazole-5-carbaldehyde (3.28 g, 34.18 mmol) were combined in DCM (75 mL) and acetic acid (2.26 mL, 39.43 mmol) was added. The suspension was stirred for 30 minutes then sodium triacetoxyborohydride (11.14 g, 52.58 mmol) was added. Stirring was continued at room temperature for 72 hours. The reaction mixture was diluted with DCM (75 mL) and washed with NaOH (2M aq., 100 mL). The aqueous wash was extracted with DCM (2×100 mL) then organics were combined, washed with brine, dried over sodium sulphate, filtered and solvent was evaporated in vacuo to afford crude product as an orange oil. Purification by flash column chromatography (silica column, DCM 0% to 10% gradient of a solution of 10% NH₄OH in MeOH) followed by concentration of the appropriate fractions in vacuo afforded N-((1H-imidazol-4-yl)methyl)-N-methyl-3-nitroaniline as an orange gum (4.19 g, 69%).

¹H NMR (400 MHz, CDCl₃) δ 7.64 (d, J=0.75 Hz, 1H), 7.57 (t, J=2.38 Hz, 1H), 7.51 (dd, J=1.51, 8.03 Hz, 1H), 7.30 (t, J=8.16 Hz, 1H), 7.05 (dd, J=2.38, 8.41 Hz, 1H), 6.85 (s, 1H), 4.57 (s, 2H), 3.11 (s, 3H).

### Synthesis of N-methyl-3-nitro-N-((1-((2-(trimethylsilyl)ethoxy)methyl)-1H imidazol-4-yl)methyl)aniline and N-methyl-3-nitro-N-((1-((2-(trimethylsilyl)ethoxy)methyl)-1H-imidazol-5-yl)methyl)aniline 1:1 Regioisomeric Mixture

NaH (60%, 1.26 g, 31.39 mmol) was suspended in THF (20 mL) placed under argon and cooled to 0° C. A solution of N-(1H-imidazol-5-ylmethyl)-N-methyl-3-nitro-aniline (6.08 g, 26.16 mmol) in THF (80 mL) was added slowly and the mixture stirred at 0° C. for 45 minutes. 2-(Trimethylsilyl)ethoxymethyl chloride (6.95 mL, 39.24 mmol) was added and the reaction stirred at 0° C. for 30 minutes and at room temperature for 2 hours. The reaction mixture was concentrated in vacuo to remove THF and the resulting suspension partitioned between EtOAc (150 mL) and NaOH (1 M, 100 mL). The aqueous layer was extracted with EtOAc (50 mL) then organics were combined, washed with brine, dried over sodium sulphate, filtered and concentrated in vacuo to afford crude product as an orange solid. Purification by flash column chromatography (silica column, DCM 0% to 10% gradient of a solution of 10% NH₄OH in MeOH) followed by evaporation of solvent from the appropriate fractions afforded a 1:1 regioisomeric mixture of the title compounds as an orange oil (6.02 g, 64%).

LC-MS Analytical Method A: rt=1.52, 1.58 min, m/z 363.2 [M+H]⁺.

### Synthesis of N¹-methyl-N¹-((1-((2-(trimethylsilyl)ethoxy)methyl)-1H-imidazol-4-yl)methyl)benzene-1,3-diamine and N¹-methyl-N¹-((1-((2-(trimethylsilyl)ethoxy)methyl)-1H-imidazol-5-yl)methyl)benzene-1,3-diamine 1:1 Regioisomeric Mixture

A solution of N-methyl-3-nitro-N-((1-((2-(trimethylsilyl)ethoxy)methyl)-1H-imidazol-4-yl)methyl)aniline and N-methyl-3-nitro-N-((1-((2-(trimethylsilyl)ethoxy)methyl)-1H-imidazol-5-yl)methyl)aniline 1:1 Regioisomeric Mixture (1.13 g, 3.12 mmol) in MeOH (100 ml) was passed through a H-Cube hydrogenation system equipped with a 10% Pd/C cartridge at 1 ml/min flow rate, 30° C. and a hydrogen pressure of 20 bar. The eluant was concentrated in vacuo to afford a brown oil. Purification by flash column chromatography (silica column, DCM 0% to 10% gradient of a solution of 10% NH₄OH in MeOH) followed by evaporation of solvent from the appropriate fractions afforded the title compound regioisomeric mixture as a yellow oil (0.86 g, 83%).

LC-MS Analytical Method A: rt=1.29 min, m/z 333.2 [M+H]⁺.

### Synthesis of N-(3-(methyl((1-((2-(trimethylsilyl)ethoxy)methyl)-1H-imidazol-4-yl)methyl)amino)phenyl) isobutyramide and N-(3-(methyl((1-((2-(trimethylsilyl)ethoxy)methyl)-1H-imidazol-5-yl)methyl)amino)phenyl) isobutyramide 1:1 Regioisomeric Mixture

N¹-methyl-N¹-((1-((2-(trimethylsilyl)ethoxy)methyl)-1H-imidazol-4-yl)methyl)benzene-1,3-diamine and N¹-methyl-N¹-((1-((2-(trimethylsilyl)ethoxy)methyl)-1H-imidazol-5-yl)methyl)benzene-1,3-diamine 1:1 regioisomeric mixture (1.36 g, 4.08 mmol) was dissolved in DCM (20 mL) and DIEA (2.13 mL, 12.23 mmol) was added. The solution was cooled to 0° C., 2-methylpropanoyl chloride (0.56 mL, 5.3 mmol) was added and the reaction was stirred at 0° C. for 1 hour. The reaction mixture was diluted with DCM (20 mL) and washed with water (20 ml) saturated sodium bicarbonate solution (20 mL) and brine. Organics were dried over sodium sulphate, filtered and solvent was evaporated in vacuo to afford a brown oil. Purification by flash column chromatography (silica column, DCM 0% to 10% gradient of a solution of 10% NH₄OH in MeOH) followed by evaporation of solvent from the appropriate fractions afforded the title compound regioisomeric mixture as a pale brown oil (1.1 g, 67%).

LCMS Analytical Method A: rt=1.49 min, m/z 403.2 [M+H]⁺.

### Synthesis of N¹-isobutyl-N³-methyl-N³-((1-((2-(trimethylsilyl)ethoxy)methyl)-1H-imidazol-4-yl)methyl)benzene-1,3-diamine and N¹-isobutyl-N³-methyl-N³-((1-((2-(trimethylsilyl)ethoxy)methyl)-1H-imidazol-5-yl)methyl)benzene-1,3-diamine 1:1 Regioisomeric Mixture

N-(3-(methyl((1-((2-(trimethylsilyl)ethoxy)methyl)-1H-imidazol-4-yl)methyl)amino)phenyl) isobutyramide and N-(3-(methyl((1-((2-(trimethylsilyl)ethoxy)methyl)-1H-imidazol-5-yl)methyl)amino)phenyl) isobutyramide 1:1 regioisomeric mixture (1.1 g, 2.73 mmol) was dissolved in 2-MeTHF (7.5 mL) and borane THF complex solution (1 M, 7.5 mL, 7.50 mmol) was added slowly at room temperature. The reaction was heated to 70° C. for 20 hours then additional borane THF complex solution (1 M, 7.5 mL, 7.50 mmol) was added and heating continued for 72 hours. MeOH (20 mL) was added and heating continued at 70° C. for 24 hours. Solvent was evaporated in vacuo and the resulting brown oil loaded onto silica and purified by flash column chromatography (silica column, DCM 0% to 10% gradient of a solution of 10% NH₄OH in MeOH). Evaporation of solvent from the appropriate fractions afforded the title compound regioisomeric mixture as a pale brown gum (0.68 g, 64%).

LC-MS Analytical Method A: rt=1.54 min, m/z 389.2 [M+H]⁺.

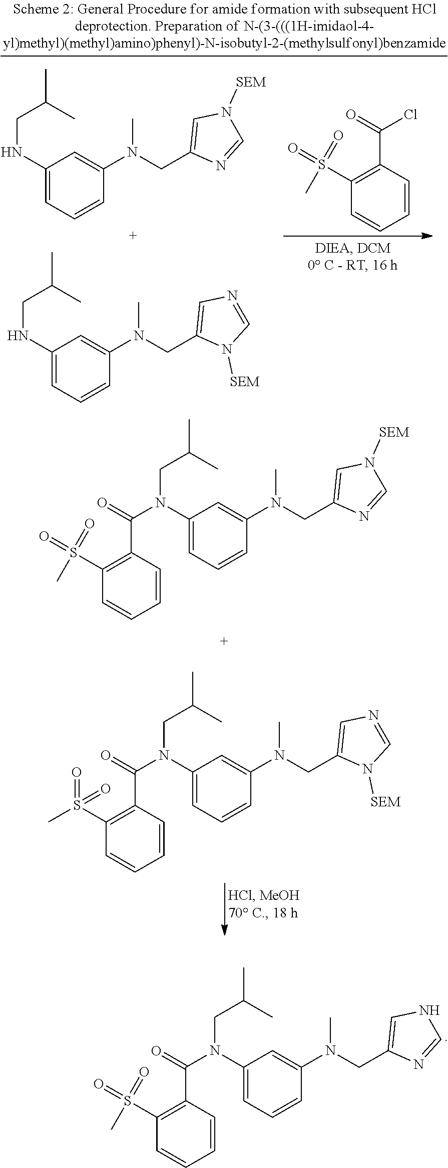

### N-isobutyl-N-(3-(methyl((1-((2-(trimethylsilyl)ethoxy)methyl)-1H-imidazol-4-yl)methyl)amino)phenyl)-2-(methylsulfonyl)benzamide and N-isobutyl-N-(3-(methyl((1-((2-(trimethylsilyl)ethoxy)methyl)-1H-imidazol-5-yl)methyl)amino)phenyl)-2-(methylsulfonyl)benzamide 1:1 Regioisomeric Mixture

N¹-isobutyl-N³-methyl-N³-((1-((2-(trimethylsilyl)ethoxy)methyl)-1H-imidazol-4-yl)methyl)benzene-1,3-diamine and N¹-isobutyl-N³-methyl-N³-((1-((2-(trimethylsilyl)ethoxy)methyl)-1H-imidazol-5-yl)methyl)benzene-1,3-diamine 1:1 regioisomeric mixture (47 mg, 0.12 mmol) was dissolved in DCM (1 mL) and DIEA (0.21 mL, 1.21 mmol. The solution was cooled to 0° C. and 2-methylsulfonylbenzoyl chloride (52.89 mg, 0.24 mmol) was added. The reaction was stirred at 0° C. for 30 minutes then 2-methylsulfonylbenzoyl chloride (52.89 mg, 0.24 mmol) was added and the reaction allowed to warm to room temperature and stirred for 16 hours. The reaction mixture was diluted with DCM (3 mL) and washed with HCl (0.5 M, 2 mL). Organics were filtered through a hydrophobic frit and solvent was evaporated in vacuo to afford crude product. Purification by flash column chromatography (silica column, DCM 0% to 2.5% gradient of a solution of 10% NH₄OH in MeOH) followed by evaporation of solvent from the appropriate fractions afforded the title compound regioisomeric mixture (60 mg, 87%).

LC-MS Analytical Method A: short method, rt=1.56 min, m/z 571.0 [M+H]⁺.

### N-(3-(((1H-imidazol-4-yl)methyl)(methyl)amino)phenyl)-N-isobutyl-2-(methylsulfonyl)benzamide

N-isobutyl-N-(3-(methyl((1-((2-(trimethylsilyl)ethoxy)methyl)-1H-imidazol-4-yl)methyl)amino)phenyl)-2-(methylsulfonyl)benzamide and N-isobutyl-N-(3-(methyl((1-((2-(trimethylsilyl)ethoxy)methyl)-1H-imidazol-5-yl)methyl)amino)phenyl)-2-(methylsulfonyl)benzamide 1:1 regioisomeric mixture (60 mg, 0.11 mmol) was dissolved in MeOH (1 mL) and HCl (5 M, 0.5 mL) added. The reaction was heated to 70° C. for 18 hours then concentrated in vacuo. The afforded residue was dissolved in DCM (2 ml) and washed with saturated sodium bicarbonate solution (2 ml). The 44queous wash was re-extracted with DCM (2 mL) then organics were combined, filtered through a hydrophobic frit and solvent was evaporated in vacuo to afford crude product, 54 mg.

Purification by preparative HPLC (basic) followed by evaporation of solvent and drying afforded N-(3-(((1H-imidazol-4-yl)methyl)(methyl)amino)phenyl)-N-isobutyl-2-(methylsulfonyl)benzamide (29 mg, 63%).

¹H NMR at 298K (400 MHz, DMSO-d₆) δ 11.81 (br. S., 1H), 7.85 (d, J=7.78 Hz, 1H), 7.52 (s, 1H), 7.37-7.49 (m, 2H), 7.14 (d, J=6.53 Hz, 1H), 6.94 (t, J=8.03 Hz, 1H), 6.83 (br. S., 1H), 6.68 (s, 1H), 6.60 (d, J=7.53 Hz, 1H), 6.44-6.53 (m, 1H), 4.23 (br. S., 2H), 3.95 (br. S., 1H), 3.41-3.56 (br. S., 1H), 3.37 (s, 3H), 2.70-2.85 (m, 3H), 1.68-1.83 (m, 1H), 0.92 (br. S., 6H).

High T NMR obtained at 373K to confirm structure:

¹H NMR at 373K (400 MHz, DMSO-d₆) δ 11.52 (br. S., 1H), 7.87 (d, J=7.28 Hz, 1H), 7.35-7.54 (m, 3H), 7.16 (br. S., 1H), 6.95 (br. S., 1H), 6.84 (s, 1H), 6.46-6.74 (m, 3H), 4.25 (br. S., 2H), 3.73 (br. S., 2H), 3.30 (s, 3H), 2.81 (br. S., 3H), 1.91 (br. S., 1H), 0.80-1.08 (m, 6H).

LC-MS Analytical Method B: rt=1.69 min, m/z 441.2 [M+H]⁺.

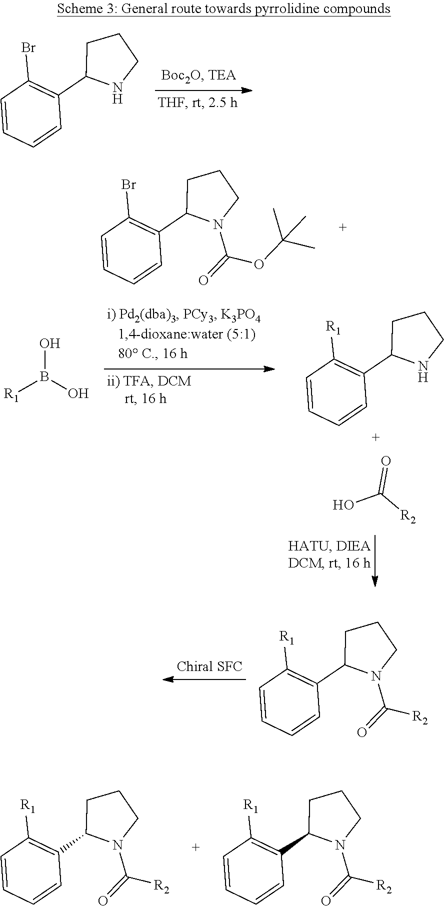

Preparation of tert-butyl 2-(2-bromophenyl)pyrrolidine-1-carboxylate

To a solution of 2-(2-bromophenyl)pyrrolidine (1500 mg, 6.63 mmol) in tetrahydrofuran (20 ml) was added di-tert-butyl dicarbonate (1737.37 mg, 7.96 mmol) and TEA (1.11 mL, 7.96 mmol) and the mixture was stirred at room temperature for 2.5 h. The mixture was reduced and charged on a 25 g silica gel cartridge. The compound was purified by flash column chromatography using heptane:EtOAc (0-100%) gradient elution. The desired fractions were reduced and collected to afford an oil 2.16 g (100%) of tert-butyl 2-(2-bromophenyl)pyrrolidine-1-carboxylate.

¹H NMR (273K, 400 MHz, CDCl₃) δ: 7.52 (dd, J=8.0, 1.2 Hz, 1H), 7.29-7.22 (m, 1H), 7.18-7.04 (m, 2H), 5.25-5.20 (m, 0.3H), 5.12 (dd, J=7.9, 4.1 Hz, 0.7H), 3.73-3.59 (m, 1.8H), 3.57-3.48 (m, 0.2H), 2.46-2.29 (m, 1H), 1.94-1.74 (m, 3H), 1.53 (s, 3H), 1.18 (s, 6H).

LC-MS Analytical Method B, RT=3.00 min, m/z 270.0, 272.0 [M+H]⁺.

Preparation of 2-(3′-methoxy-[1,1′-biphenyl]-2-yl)pyrrolidine

To a microwave vial were added tert-butyl 2-(2-bromophenyl)pyrrolidine-1-carboxylate (2440 mg, 7.48 mmol), (3-methoxyphenyl) boronic acid (1704.79 mg, 11.22 mmol), 1,4-dioxane (5 ml) and water (1 ml) then the mixture was bubbled with argon for ˜5 min. A mixture of Pd₂(dba)₃ (172.6 mg, 0.18 mmol), PCy₃ (126.8 mg, 0.45 mmol), and K₃PO₄ (1600 mg, 7.5 mmol), was added to the reaction mixture and the vessel was sealed before heating at 80° C. overnight. The mixture was partitioned between water and EtOAc and extracted with EtOAc (3×25 ml). The organic phases were dried over Na₂SO₄ and filtered through a plug of Celite before reducing it to an oil. The oil was diluted with dichloromethane (5 ml), and TFA (5 ml, 65.34 mmol) was added to the reaction mixture before stirring it at room temperature for 2 h. The mixture was washed with water and reduced after drying it over Na₂SO₄. The crude mixture was charged on a 25 g silica cartridge and purified using DCM:MeOH (0-10%) on an Isolera. The desired fraction were reduced to obtain a yellow oil, 2560 mg (93.2%) of 2-[2-(3-methoxyphenyl)phenyl]pyrrolidin-1-ium with a trace of residual trifluoroacetic acid.

¹H NMR (273K, 400 MHz, CDCl₃) δ: 10.25 (br, 1H); 8.95 (br, 1H); 7.70 (m, 1H); 7.31-7.27 (m, 4H); 7.26-7.22 (m, 1H); 6.93 (dd, J=8.4, 2.6 Hz, 1H); 6.79-6.73 (m, 1H); 4.60-4.48 (m, 1H); 3.83 (s, 3H); 3.40 (m, 1H); 3.16-3.04 (m, 1H); 2.27-2.12 (m, 2H); 2.10-2.02 (m, 1H); 1.99-1.88 (m, 1H).

LC-MS Analytical Method B, RT=1.58 min, m/z 254.0 [M+H]⁺.

Preparation of (3-chlorothiophen-2-yl)(2-(3′-methoxy-[1,1′-biphenyl]-2-yl)pyrrolidin-1-yl)methanone

To a vial were added 2-[2-(3-methoxyphenyl)phenyl]pyrrolidine (50.67 mg, 0.2 mmol), 3-chlorothiophene-2-carboxylic acid (42 mg, 0.26 mmol), dichloromethane (4 ml), DIEA (0.09 ml, 0.5 mmol) and O-(7-azabenzotriazol-1-yl)-N,N,N′,N′-tetramethyluronium hexafluorophosphate (114.07 mg, 0.3 mmol) then the mixture was stirred at room temperature overnight. The solvents were removed by centrifugal evaporation, then purified on preparative HPLC (acidic conditions). The desired fractions were collected and evaporated to give the title compound, 34.5 mg (9%).

¹H NMR (273K, 400 MHz, DMSO-d₆) δ: 7.64 (d, J=4.8 Hz, 1H); 7.40-7.30 (m, 3H); 7.27 (td, J=7.3, 1.8 Hz, 1H); 7.1 (d, J=7.3 Hz, 1H); 6.99-6.92 (m, 2H); 6.75 (br, 1H); 5.13 (dd, J=7.5, 5.4 Hz, 1H); 3.89-3.79 (m, 4H); 3.67-3.57 (m, 1H); 2.23-2.11 (m, 1H); 2.04-1.93 (m, 1H); 1.90-1.72 (m, 2H);

LC-MS Analytical Method B, RT=2.71 min, m/z 398.2 [M+H]⁺.

Preparation of (3-chlorothiophen-2-yl)(2-(3′-hydroxy-[1,1′-biphenyl]-2-yl)pyrrolidin-1-yl)methanone

To a solution of (3-chlorothiophen-2-yl)(2-(3′-methoxy-[1,1′-biphenyl]-2-yl)pyrrolidin-1-yl)methanone (50 mg, 0.126 mmol) in DCM at −78° C. was added 1 M BBr₃ in dichloromethane (0.09 ml). The mixture was left to slowly warm to room temperature, a further portion of 1 M BBr₃, 0.1 ml, was then added to the reaction mixture and stirred overnight. After 16 h a further portion of 1 M BBr₃ (0.1 ml) was added and the mixture left to stir for a further 5 hours at room temperature. The product was quenched with 10 ml of 1M HCl and extracted with DCM (3×20 ml). The organic layers were dried over a phase separator and reduced. The crude material was charged on a 10 g silica cartridge and purified flash column chromatography Heptane:EtOAc as a gradient elution. The desired fractions were reduced and dried to obtain the title compound as a white powder, 42 mg (85% yield).

¹H NMR (373K, 400 MHz, DMSO-d₆) δ: 9.05 (1H, OH, s); 7.62 (d, J=4.4 Hz, 1H); 7.37-7.27 (m, 2H); 7.26-7.15 (m, 2H); 7.06 (d, J=7.3 Hz, 1H); 6.96 (d, J=5.2 Hz, 1H); 6.78 (dd, J=7.8, 1.8 Hz, 1H); 6.60 (br, 2H); 5.18-5.09 (m, 1H); 3.87-3.76 (m, 1H); 3.68-3.57 (m, 1H); 2.23-2.14 (m, 1H); 2.02-1.92 (m, 1H); 1.88-1.69 (m, 2H);

LC-MS Analytical Method B, RT=2.56 min, m/z 384.2 [M+H]⁺.

### Separation of Enantiomer

Preparation of(S)-(3-chlorothiophen-2-yl)(2-(3′-hydroxy-[1,1′-biphenyl]-2-yl)pyrrolidin-1-yl)methanone

The compound was obtained by separation of a racemic mixture using a Waters Investigator supercritical fluid (SFC). Separation was carried out on a Chiracel AD-H column 10×250 mm, using a flow rate of 10 ml/min, an injection volume 100 microlitres (concentration 10 mg/mL) and 13% methanol as a co-solvent. After repeated injections the appropriate fractions were collected, methanol was evaporated to provide the title compound as a solid.

¹H NMR (373K, 400 MHz, DMSO-d₆) δ: 9.05 (1H, OH, s); 7.62 (d, J=4.4 Hz, 1H); 7.37-7.27 (m, 2H); 7.26-7.15 (m, 2H); 7.06 (d, J=7.3 Hz, 1H); 6.96 (d, J=5.2 Hz, 1H); 6.78 (dd, J=7.8, 1.8 Hz, 1H); 6.60 (br, 2H); 5.18-5.09 (m, 1H); 3.87-3.76 (m, 1H); 3.68-3.57 (m, 1H); 2.23-2.14 (m, 1H); 2.02-1.92 (m, 1H); 1.88-1.69 (m, 2H);

LC-MS Analytical Method B, RT=2.56 min, m/z 384.2 [M+H]⁺.

Chiral SFC (AD-H column, 5 ml/min, 13% MeOH), RT=6.64 min, 99% ee

### Synthetic Procedures for Compounds Referred to in Table 6

### Procedure 1 (ESC1002019)

HCl (4M in dioxane, 1 ml) was added to tert-butyl 4-(2-carbamoyl-1-propyl-indol-5-yl) pyrazole-1-carboxylate (34 mg, 0.09 mmol) and the resulting suspension stirred for 18 hours (LCMS). Solvent was evaporated under reduced pressure and the resulting residue partitioned between EtOAc (20 ml) and saturated sodium bicarbonate solution (20 ml). Organics were washed with brine and solvent was evaporated under reduced pressure to afford a pale brown solid (35 mg). The solid was combined with product (12 mg) for purification. Purification by prep. HPLC (basic) followed by evaporation of solvent from the appropriate fractions afforded product 5916A, 24 mg.

### Procedure 2 (ESC1002024)

tert-butyl 4-(4,4,5,5-tetramethyl-1,3,2-dioxaborolan-2-yl) pyrazole-1-carboxylate (49.59 mg, 0.17 mmol) was charged to a microwave vial with CatKit (‘dtbpf’ 78 mg comprising 0.05 equiv. Pd(OAc)₂, 0.1 equiv. dtbpf, 3 equiv. K₃PO₄). A solution of 2-bromo-5-(2,3,4-trimethoxyphenyl)thiophene (37 mg, 0.11 mmol) in dioxane (1.0 ml) was added followed by water (0.2 ml). The vial was sealed, placed under argon and the reaction mixture de-gassed with a flow of argon for 10 minutes. The reaction was heated to 80° C. for 16 hours (LCMS). The reaction mixture was allowed to cool and diluted with EtOAc (10 ml) and water (10 ml). Organics were washed with water (2×10 ml) and brine, dried over sodium sulphate, filtered and solvent was evaporated under reduced pressure to afford crude product (40 mg). The crude product was dissolved in HCl (4M in dioxane, 2 ml) and stirred at room temperature for 5 hours. The solvent was evaporated under reduced pressure and the resulting residue dissolved in EtOAc (10 ml) and washed with saturated sodium bicarbonate solution (10 ml). Organics were dried over sodium sulphate, filtered and solvent was evaporated under reduced pressure to afford crude product (38 mg). Purification by prep. HPLC (basic) followed by evaporation of solvent from the appropriate fractions afforded product as a white solid, 16 mg (45%) of 4-[5-(2,3,4-trimethoxyphenyl)-2-thienyl]-1H-pyrazole.

### Procedure 3 (ESC1002032)

tert-butyl 5-[[3-(diisobutylamino)-N-methyl-anilino]methyl]imidazole-1-carboxylate (37 mg, 0.09 mmol) was dissolved in HCl (4M in dioxane, 2 ml) and the reaction stirred at room temperature for 4 hours (LCMS). The reaction mixture was concentrated under reduced pressure then the residue dissolved in EtOAc (10 ml) and washed with saturated sodium bicarbonate solution (10 ml). Organics were dried over sodium sulphate, filtered and solvent was evaporated under reduced pressure to afford crude product (28 mg). Purification by prep. HPLC (basic) followed by evaporation of solvent from the appropriate fractions afforded product as an off-white solid, 12 mg (42.8%) of N3-(1H-imidazol-5-ylmethyl)-N1,N1-diisobutyl-N3-methyl-benzene-1,3-diamine.

### Procedure 4 (ESC1002033)

tert-butyl 5-[[3-(isobutylamino)-N-methyl-anilino]methyl]imidazole-1-carboxylate (85%, 65 mg, 0.15 mmol), 2-fluorobenzoic acid (32.39 mg, 0.23 mmol) and O-(7-Azabenzotriazol-1-yl)-N,N,N′,N′-tetramethyluronium hexafluorophosphate (87.9 mg, 0.23 mmol) were charged to a reaction tube and dichloromethane (2 ml) added followed by DIEA (0.07 ml, 0.39 mmol). The reaction was stirred at room temperature for 16 hours. The reaction mixture was diluted with DCM (5 ml) and washed with water (2 ml). Organics were filtered through a hydrophobic frit and solvent was evaporated under reduced pressure to afford crude product (134 mg). Purification by flash chromatography (silica column heptane 10% to 60% EtOAc gradient) followed by evaporation of solvent from the appropriate fractions afforded product 5923A (26 mg, Boc-protected). Further eluting the column with a solution of DCM:MeOH:NH4OH (180:10:1) followed by evaporation of solvent from the appropriate fractions afforded product 5923C (6 mg, de-protected, LCMS). Product 5923A was dissolved in HCl (4M diox., 1 ml) and stirred at room temperature for 4 hours, when no protected material remained. Solvent was evaporated under reduced pressure then the resulting residue dissolved in EtOAc (10 ml) and washed with saturated sodium bicarbonate solution (10 ml). Organics were dried over sodium sulphate, filtered and solvent was evaporated under reduced pressure to afford crude product 5923D (22 mg). Products 5923C and 5923D were combined for purification by prep. HPLC (basic). Evaporation of solvent from the appropriate fractions and drying under vacuum afforded product as a colourless glass, 20 mg (34.1%) of 2-fluoro-N-[3-[1H-imidazol-5-ylmethyl(methyl)amino]phenyl]-N-isobutyl-benzamide.

### ESC1002032

Procedure 4 with 4-methylbenzoic acid (31.48 mg, 0.23 mmol) in place of 2-fluorobenzoic acid and additional 4-methylbenzoic acid (15 mg)O-(7-Azabenzotriazol-1-yl)-N,N,N′,N′-tetramethyluronium hexafluorophosphate (44 mg) and DIEA (40 μl) added after 16 hours, with stirring continued for 5 days. The reaction mixture was diluted with DCM (5 ml) and washed with 0.5M HCl (aq., 5 ml). Crude product (258 mg). Flash chromatography (silica column, heptane 0% to 100% EtOAc gradient) afforded product 5922A (42 mg, boc-protected product, contains tetramethylurea impurity). 5922A was dissolved in HCl (4M in dioxane, 1 ml), stirred at room temperature for one hour and allowed to stand for 16 hours. Crude product (29 mg) was extracted as in procedure D. Purification by prep. HPLC (basic method) followed by evaporation of solvent from the appropriate fractions under reduced pressure afforded product as a white solid, 23 mg (39.6%) of N-[3-[1H-imidazol-5-ylmethyl(methyl)amino]phenyl]-N-isobutyl-4-methyl-benzamide

### Procedure 5 (ESC1002038)

tert-butyl 5-[[3-(isobutylamino)-N-methyl-anilino]methyl]imidazole-1-carboxylate (65 mg, 0.18 mmol) was dissolved in DCM (1 ml) and DIEA (0.08 ml, 0.45 mmol) added. The solution was cooled to 0° C. then a solution of 3-methoxybenzoyl chloride (46.4 mg, 0.27 mmol) in DCM (0.5 ml) was added. The reaction was allowed to warm to room temperature and stirred for 16 hours (LCMS). The reaction mixture was partitioned between DCM (5 ml) and HCl (aq. 0.5M, 5 ml) then organics were filtered through a hydrophobic frit and solvent was evaporated to afford crude intermediate product (121 mg). Purification by flash chromatography (silica column, heptane 0% to 100% EtOAc gradient) followed by evaporation of solvent afforded two products, 5933A and 5933B. Product 5933A consisting of mainly a side product but also some desired boc-protected product present, product 5933B being mainly desired boc-protected product. Each intermediate product was separately dissolved in HCl (4M in dioxane, 2 ml) stirred at room temperature for one hour and allowed to stand for 16 hours. Solvent was evaporated from each under reduced pressure then each residue was dissolved in EtOAc (5 ml) and washed with saturated sodium bicarbonate solution (2 ml). Organics were dried over sodium sulphate, filtered and solvent was evaporated under reduced pressure to afford a yellow oil in each case. 5933A afforded 38 mg (LCMS) and 5933B afforded 26 mg (LCMS). Both residues were purified by prep. HPLC (basic). Product fractions from each were combined and solvent was evaporated under reduced pressure to afford product as a pale yellow solid, 20 mg (28.1%) of N-[3-[1H-imidazol-5-ylmethyl(methyl)amino]phenyl]-N-isobutyl-3-methoxy-benzamide.

### (ESC1002034)

Procedure 5 with pyridine-4-carbonyl chloride hydrochloride (48.42 mg, 0.27 mmol) in place of a solution of 3-methoxybenzoyl chloride. Crude intermediate product (64 mg, LCMS), mainly boc-protected desired product). The aqueous wash was basified with addition of NaOH then extracted with DCM (2×3 ml). Organics were combined, filtered through a hydrophobic frit and solvent was evaporated under reduced pressure to afford a second crude product (26 mg, LCMS, mainly de-protected desired product). The first (boc-protected) crude product was isolated as a yellow oil (LCMS) as described in procedure E for the intermediate products. The two crude residues were purified by prep. HPLC (basic) to afford product as a pale yellow solid, 17 mg (25.8%) of N-[3-[1H-imidazol-5-ylmethyl(methyl)amino]phenyl]-N-isobutyl-pyridine-4-carboxamide.

### ESC1002042

Procedure 5 with 4-chloro-2-fluoro-benzoyl chloride (52.49 mg, 0.27 mmol) in place of 3-methoxybenzoyl chloride. Crude intermediate product (114 mg). Purification by flash chromatography (silica column, heptane 0% to 50% EtOAc gradient) afforded two products, 5932A and 5932B. Product 5932A consisting of mainly a side product but also some desired boc-protected product present, product 5932B (LCMS) being mainly desired boc-protected product. Each intermediate product was isolated as a yellow oil following the methods described in procedure E. 5932A afforded 26 mg (LCMS) and 5932B afforded 33 mg (LCMS). Both residues were purified by prep. HPLC to afford product as a pale yellow solid, 28 mg (37.2%) of 4-chloro-2-fluoro-N-[3-[1H-imidazol-5-ylmethyl(methyl)amino]phenyl]-N-isobutyl-benzamide.

### Procedure 6 (ESC1002054)

Procedure 4 with tert-butyl 5-[(3-amino-N-methyl-anilino)methyl]imidazole-1-carboxylate (89%, 60 mg, 0.18 mmol), O-(7-Azabenzotriazol-1-yl)-N,N,N′,N′-tetramethyluronium hexafluorophosphate (100.72 mg, 0.26 mmol), and 4-methylbenzoic acid (36.07 mg, 0.26 mmol). The reaction mixture was diluted with DCM (5 ml) and washed with 0.5M HCl (aq., 2.5 ml) instead of water. Crude intermediate was isolated as a brown oil. The oil was dissolved in HCl (4M in dioxane, 1.5 ml) and stirred at room temperature for 2 hours (LCMS). Solvent was evaporated under reduced pressure and the resulting residue dissolved in DCM (5 ml) and washed with saturated sodium bicarbonate solution (5 ml). Organics were filtered through a hydrophobic frit and solvent was evaporated under reduced pressure to afford crude product. Purification by prep. HPLC (basic) followed by evaporation of solvent from the appropriate fractions afforded product as a pale yellow solid, 18 mg (31.8%) of N-[3-[1H-imidazol-5-ylmethyl(methyl)amino]phenyl]-4-methyl-benzamide.

### ESC1002056

Procedure F with 2-fluorobenzoic acid (37.12 mg, 0.26 mmol) in place of 4-methylbenzoic acid. Product isolated as a pale yellow solid, 23 mg (40.2%) of 2-fluoro-N-[3-[1H-imidazol-5-ylmethyl(methyl)amino]phenyl]benzamide.

### Procedure 7 (ESC1002065

N-benzyl-2-chloro-5-(morpholinomethyl)pyrimidin-4-amine (178 mg, 0.56 mmol), 4-amino-2-chloro-phenol (96.19 mg, 0.67 mmol), NaHCO₃ (234.52 mg, 2.79 mmol) and tetrahydrofuran (10 ml), were added to a flask and the mixture was stirred at 60° C. overnight. The mixture was reduced, and partitioned between DCM and Water and further extracted with DCM (3×15 ml). The organic phases were dried over Na₂SO₄. The solvents were removed and further 4-amino-2-chloro-phenol (96.19 mg, 0.67 mmol) was added to the reaction mixture followed by dioxane (40 ml) and the mixture was heated to 100° C. for 3 h. The mixture was reduced and loaded on a 25 g silica gel cartridge. The compound was purified on an Isolera using Hep:EtOAc (0-100%). The desired fractions were collected, reduced and the oil was further purified by Prep HPLC (basic). The collected fractions were dried, diluted in EtOAc and washed with 2M NaOH solution (3×25 ml). The organic layer was dried over Na₂SO₄ and the solvents were removed.

### Procedure 8 (ESC1002078)

tert-butyl 5-[[3-(isopropylamino)-N-methyl-anilino]methyl]imidazole-1-carboxylate (45 mg, 0.13 mmol) was dissolved in DCM (2 ml) and DIEA (0.07 ml, 0.39 mmol) was added. The solution was cooled to 0° C. then a solution of 2-fluorobenzoyl chloride (31.07 mg, 0.2 mmol) in DCM (0.5 ml) was added dropwise. The reaction was allowed to warm to room temperature and stirred for one hour, then allowed to stand for 16 hours. The reaction mixture was diluted with DCM (5 ml) and washed with saturated sodium bicarbonate solution (5 ml). Organics were filtered through a hydrophobic frit and solvent was evaporated under reduced pressure to afford crude intermediate as a yellow gum. The intermediate was dissolved in HCl (4M in dioxane, 1.5 ml) and stirred for 3 hours (LCMS). Solvent was evaporated under reduced pressure to afford a yellow gum. Purification by prep. HPLC (basic) followed by evaporation of solvent from the appropriate fractions and drying under vacuum afforded product, 18 mg (37.6%) of 2-fluoro-N-[3-[1H-imidazol-5-ylmethyl(methyl)amino]phenyl]-N-isopropyl-benzamide.

### ESC1002080

Procedure 8 with 4-methylbenzoyl chloride (30.29 mg, 0.2 mmol) in place of 2-fluorobenzoyl chloride. Product isolated as 28 mg (59.1%) of N-[3-[1H-imidazol-5-ylmethyl(methyl)amino]phenyl]-N-isopropyl-4-methyl-benzamide.

### Procedure 9 (ESC1002081)

(7-amino-3,4-dihydro-2H-quinolin-1-yl)-(2-fluorophenyl)methanone (35 mg, 0.13 mmol) and 1H-imidazole-5-carbaldehyde (14.93 mg, 0.16 mmol) were suspended in DCM (1.5 ml) and acetic acid (0.01 ml, 0.19 mmol) was added. The mixture was stirred for 15 minutes then sodium triacetoxyborohydride (54.89 mg, 0.26 mmol) was added. Stirring was continued at room temperature for 16 hours (LCMS). The reaction mixture was diluted with DCM (20 ml) and washed with NaOH (1M aq., 10 ml) and brine (10 ml). Organics were dried over sodium sulphate, filtered and solvent was evaporated under reduced pressure to afford crude product as an off white gum (74 mg). Purification by prep. HPLC (basic) followed by evaporation of solvent from the appropriate fractions and drying under vacuum afforded product, 20 mg (44.1%) of (2-fluorophenyl)-[7-(1H-imidazol-5-ylmethylamino)-3,4-dihydro-2H-quinolin-1-yl]methanone.

### Procedure 10 (ESC1002082)

[2-(2-bromophenyl)pyrrolidin-1-yl]-(3-methyl-2-thienyl)methanone (110 mg, 0.31 mmol) (6-methoxy-3-pyridyl) boronic acid (57.64 mg, 0.38 mmol) and ‘first choice’ catkit (158 mg consisting of Pd₂(dba)₃ 0.05 equiv., P(cy)₃ 0.2 equiv., and K₃PO₄ 2 equiv.) were charged to a sealable vial. 1,4-Dioxane (1 ml) was added followed by water (0.2 ml). The vial was sealed and placed under argon then the solution was de-gassed with a flow of argon for 10 minutes. The reaction was heated to 80° C. for 20 hours (LCMS t=5 hours, LCMS t=20 hours). The reaction mixture was diluted with EtOAc (5 ml) and water (2 ml) and filtered through celite. Organics were separated, washed with water (5 ml) and brine, dried over sodium sulphate, filtered and solvent was evaporated under reduced pressure to afford crude product, 126 mg. Purification by flash chromatography (silica column, heptane 0% to 100% gradient) followed by evaporation of solvent from the appropriate fractions under reduced pressure and drying under vacuum afforded product, 80 mg (67.3%) of [2-[2-(6-methoxy-3-pyridyl)phenyl]pyrrolidin-1-yl]-(3-methyl-2-thienyl)methanone.

ESC1002161 and ESC1002162 were obtained by SFC chiral separation of ESC1002082.

### ESC1002083

Procedure 9 with (2-fluorophenyl)-[7-(methylamino)-3,4-dihydro-2H-quinolin-1-yl]methanone (130 mg, 0.46 mmol), 1H-imidazole-5-carbaldehyde (52.72 mg, 0.55 mmol) and acetic acid (0.04 ml, 0.69 mmol) and sodium triacetoxyborohydride (193.8 mg, 0.91 mmol). Crude product, 190 mg. Product was isolated as 71 mg (42.6%) of (2-fluorophenyl)-[7-[1H-imidazol-5-ylmethyl(methyl)amino]-3,4-dihydro-2H-quinolin-1-yl]methanone.

### Procedure 11 (ESC 1002089, ESC1002090, ESC1002091 and ESC1002092)

Suzuki cross coupling: aryl bromide, the desired boronic acid, the solvents (dioxane+water) and the mixture was added to a microwave vial and bubbled with argon for ˜5 min. Then the catkit first choice was added to the reaction mixture and the vessel was sealed before heating it to 80° C. for the required time. Once completed the mixture was diluted with EtOAc and Water and extracted with EtOAc (3×15 ml). The org. layers were dried over Na₂SO₄, and reduced before charging the mixture on a 10 g silica cartridge. The mixture was purified on an Isolera and the desired fractions were collected. The solids obtained were further dried in a vac. oven (50° C. overnight).

ESC1002331 and ESC1002332 were obtained by SFC chiral separation of ESC1002089.

### ESC1002119

Procedure 10 with [2-(2-bromophenyl)pyrrolidin-1-yl]-(2-fluorophenyl)methanone (85.0 mg, 0.244 mmol), (6-methoxy-3-pyridyl) boronic acid (44.8 mg, 0.293 mmol) and first choice catkit (127 mg, 0.252 mmol). The desired compound was obtained as a yellow white in solid 49.1 mg (53.5%) of (2-fluorophenyl)-[2-[2-(6-methoxy-3-pyridyl)phenyl]pyrrolidin-1-yl]methanone

### ESC1002120

Procedure 10 with [2-(2-bromophenyl)pyrrolidin-1-yl]-(p-tolyl)methanone (65.0 mg, 0.189 mmol), (6-methoxy-3-pyridyl) boronic acid (34.7 mg, 0.227 mmol) and first choice catkit (98.0 mg, 0.194 mmol). The desired compound was obtained as a yellow-white solid in 40.8 mg (58.0%) of [2-[2-(6-methoxy-3-pyridyl)phenyl]pyrrolidin-1-yl]-(p-tolyl)methanone.

### Procedure 12 (ESC1002163 and ESC1002164)

tert-butyl 5-[[3-[(2-fluorobenzoyl)amino]-N-methyl-anilino]methyl]imidazole-1-carboxylate (44 mg, 0.1 mmol) was dissolved in DMF (1 ml) and NaH (60%, 4.98 mg, 0.12 mmol) was added. The reaction was stirred at room temperature for 20 minutes. 1-bromo-2-methoxy-ethane (0.01 ml, 0.1 mmol) was added and stirring continued at room temperature for 4 hours then the reaction was heated to 60° C. for 16 hours (LCMS). Additional NaH (60%, 4.98 mg, 0.12 mmol) and 1-bromo-2-methoxy-ethane (0.01 ml, 0.1 mmol) were added and heating to 60° C. was continued for 5 hours. The reaction was allowed to cool to room temperature. The reaction mixture was diluted with EtOAc (20 ml) and washed with water (3×20 ml) and brine (10 ml). Organics were dried over sodium sulphate, filtered and solvent was evaporated under reduced pressure to afford crude product as a pale yellow gum, 59 mg. Purification by prep. HPLC (basic, ‘late’ focussed gradient) followed by evaporation of solvent under reduced pressure and drying afforded products EXP-16-FB5962A and EXP-16-FB5962B. EXP-16-FB5962A product of alkylation on imidazole. Regiochemistry of addition confirmed by NMR analysis. Some residual DCM still present so the product was dried under vacuum to afford final product EXP-16-FB5962C, confirmed as ESC1002163. EXP-16-FB5962B product of double alkylation: 4 mg (8.8%) of 2-fluoro-N-(2-methoxyethyl)-N-[3-[[1-(2-methoxyethyl)imidazol-4-yl]methyl-methylamino]phenyl]benzamide. Product EXP-16-FB5962B confirmed as ESC1002164-01.

### Procedure 13 (ESC1002165)

tert-butyl 5-[[3-(isobutylamino)-N-methyl-anilino]methyl]imidazole-1-carboxylate (45 mg, 0.13 mmol) was dissolved in DCM (1 ml) and DIEA (0.11 ml, 0.63 mmol) was added. The solution was cooled in an ice-bath and 2-methylpropanoyl chloride (0.02 ml, 0.19 mmol) was added. The reaction was allowed to warm to room temperature and stirred for 3 hours (LCMS). The reaction mixture was diluted with DCM (5 ml) and washed with water (2 ml). Organics were filtered through a hydrophobic frit and solvent was evaporated to afford crude intermediate. The intermediate was dissolved in HCl (4M diox., 1 ml) and stirred at room temperature for 3 hours. Solvent was evaporated under reduced pressure. The afforded residue was dissolved in 1:1 DMSO/MeOH and sodium carbonate added. The suspension was stirred for 30 minutes and filtered. Purification by prep. HPLC (basic) followed by evaporation of solvent from the appropriate fractions. The resulting residues were dissolved in DCM and combined in a vial. Solvent was evaporated and the resulting gum dried under vacuum. The afforded gum was difficult to handle so was dissolved in MeOH/water then solvent was evaporated (genevac, HPLC lyophilisation program). The afforded pale yellow gum was dried under vacuum to afford product, 23 mg (55.8%) of N-[3-[1H-imidazol-5-ylmethyl(methyl)amino]phenyl]-N-isobutyl-2-methyl-propanamide.

### ESC1002166

Procedure 13 with cyclohexanecarbonyl chloride (0.03 ml, 0.19 mmol) in place of 2-methylpropanoyl chloride. The product was obtained as 27 mg (58.4%) of N-[3-[1H-imidazol-5-ylmethyl(methyl)amino]phenyl]-N-isobutyl-cyclohexanecarboxamide.

### ESC1002167

Procedure 13 with 2-phenylacetyl chloride (0.02 ml, 0.19 mmol) in place of 2-methylpropanoyl chloride. The product was obtained as a pale yellow gum, 25 mg (52.9%) of N-[3-[1H-imidazol-5-ylmethyl(methyl)amino]phenyl]-N-isobutyl-2-phenyl-acetamide.

### ESC1002168

Procedure 13 with 3-phenylpropanoyl chloride (0.03 ml, 0.19 mmol) in place of 2-methylpropanoyl chloride. The product was obtained as a pale yellow gum, 39 mg (79.6%) of N-[3-[1H-imidazol-5-ylmethyl(methyl)amino]phenyl]-N-isobutyl-3-phenyl-propanamide.

### ESC1002169

Procedure 13 with benzoyl chloride (0.02 ml, 0.19 mmol) in place of 2-methylpropanoyl chloride. The product was obtained as a yellow gum, 26 mg (57.1%) of N-[3-[1H-imidazol-5-ylmethyl(methyl)amino]phenyl]-N-isobutyl-benzamide.

### Procedure 14 (ESC 10021710)

1-(2-methylbenzofuran-3-yl)-2-phenyl-ethanone (110 mg, 0.44 mmol) was suspended in ethanol (2 ml) and NH₂NH₂·H₂O (0.04 ml, 0.88 mmol) was added. The reaction was sealed in a vial and heated to 60° C. for 2 hours (LCMS) then allowed to stand at room temperature for 16 hours. Solvent was evaporated under reduced pressure and the resulting residue partitioned between EtOAc (15 ml) and water (10 ml). Organics were washed with brine, dried over sodium sulphate, filtered and solvent was evaporated under reduced pressure to afford crude product, 140 mg. Purification by flash chromatography (silica column, heptane 10% to 100% EtOAc gradient) followed by evaporation of solvent from the appropriate fractions and drying under vacuum afforded product 5978A as a sticky white solid, 34 mg (note-yield sacrificed for purity). NMR analysis (1H, HSQC, NOE) confirmed desired product formation. Weighing the solid was problematic so the solid was dissolved in MeOH/water, transferred to a vial and the solution concentrated under reduced pressure (genevac, HPLC lyophilisation program) and dried under vacuum to afford product, 21 mg (18.1%) of 2-(5-benzyl-3-methyl-1H-pyrazol-4-yl) phenol.

### ESC1002171

Procedure 13 with acetyl chloride (0.01 ml, 0.18 mmol) in place of 2-methylpropanoyl chloride. The product was isolated as a yellow gum, 22 mg (62.5%) of N-[3-[1H-imidazol-5-ylmethyl(methyl)amino]phenyl]-N-isobutyl-acetamide.

### ESC1002172

Procedure 13 with naphthalene-1-carbonyl chloride (0.03 ml, 0.19 mmol) in place of 2-methylpropanoyl chloride. The product was isolated as a pale yellow solid, 22 mg (42.5%) of N-[3-[1H-imidazol-5-ylmethyl(methyl)amino]phenyl]-N-isobutyl-naphthalene-1-carboxamide.

### ESC1002182

Procedure 13 with 4-methoxybenzoyl chloride (32.12 mg, 0.19 mmol) in place of 2-methylpropanoyl chloride. ¹H NMR/LCMS indicated product was not pure. Purification by prep. HPLC (basic, ‘late’ focussed gradient method) followed by evaporation of solvent from the appropriate fractions afforded the product as a sticky gum. The gum was dissolved in EtOH and water and solvent evaporated (genevac, lyophilisation program) to afford a solid. Drying the solid under vacuum caused the solid to melt and form a gum-product, 14 mg (28.4%) of N-[3-[1H-imidazol-5-ylmethyl(methyl)amino]phenyl]-N-isobutyl-4-methoxy-benzamide.

### ESC1002183

Procedure 13 with 4-chlorobenzoyl chloride (0.02 ml, 0.19 mmol) in place of 2-methylpropanoyl chloride. The product was isolated as 9 mg (18.1%) of 4-chloro-N-[3-[1H-imidazol-5-ylmethyl(methyl)amino]phenyl]-N-isobutyl-benzamide.

### Procedure 15 (ESC1002199 to ESC1002214)

2-fluoro-N-isobutyl-N-[3-(methylamino)phenyl]benzamide (198.25 mg, 0.66 mmol) was added to a vial containing the desired aldehyde (24.9 mg, 0.86 mmol) and dissolved in CH₃CO₂H (1 ml, 17.48 mmol). To the mixture was added (CH₃COO)₃BHNa (209.82 mg, 0.99 mmol). The mixture was stirred overnight at rt. The solutions were quenched with 2M NaOH (2 ml) and extracted with EtOAc (3×10 ml). The organic layers were dried over Na₂SO₄ then reduced. Some compounds were purified on an Isolera (Biotage) whereas other were purified on a basic prep. Most compounds were gums, therefore it was decided to salt them by addition of 2 ml Et₂O and 1.2 eq of 4M HCl in Dioxane. Most compounds seems to have salted successfully, although some showed sign of degradation. Therefore, they were re-purified by prep HPLC (basic) and submitted as free base.

### Procedure 16 (ESC1002234)

tert-butyl 5-[[3-[(2-fluorobenzoyl)amino]-N-methyl-anilino]methyl]imidazole-1-carboxylate (114 mg, 0.27 mmol) was dissolved in DMF (1 ml) and cooled to 0° C. NaH (60%, 12.89 mg, 0.32 mmol) was added and the mixture stirred for 15 minutes. Ethyliodide (0.03 ml, 0.34 mmol) was added then the reaction mixture placed under argon, stirred at 0° C. for 10 minutes and then allowed to warm to room temperature. DMF (1 ml) was added to aid stirring and the reaction stirred for 80 hours (LCMS t=5 hours, t=80 hours. The reaction mixture was partitioned between EtOAc (30 ml) and water (20 ml). Organics were separated, washed with water (2×20 ml) and brine (10 ml) dried over sodium sulphate, filtered and solvent was evaporated under reduced pressure to afford crude product. The crude product was partially deprotected so the residue was dissolved in HCl (4M in dioxane, 2 ml) and stirred at room temperature for 3 hours to afford complete deprotection. Solvent was evaporated under reduced pressure and the resulting residue dissolved in 1:1 DMSO/MeOH. Sodium carbonate was added and the mixture stirred for 30 minutes and then filtered. Purification of the resulting solution by prep. HPLC (basic, late focussed gradient, multiple very small quantity injections required for peak separation) followed by evaporation of solvent from the appropriate fractions afforded product, 25 mg. Analytical data consistent with desired product (¹H NMR at 298K, ¹⁹F NMR and COSY NMR, LCMS; ¹H NMR at 373K but product contains some EtOAc residue. Product was dissolved in EtOH and water and solvent was evaporated (genevac, lyophilisation program). The afforded solid was dried under vacuum to afford product, 20 mg, ¹H NMR indicates no EtOAc but other aliphatic impurities are present. Purification by prep. HPLC (basic, late focussed gradient) followed by evaporation of solvent from the appropriate fractions and drying under vacuum afforded product, 14 mg (14.8%) of N-ethyl-2-fluoro-N-[3-[1H-imidazol-5-ylmethyl(methyl)amino]phenyl]benzamide.

### Procedure 17 (ESC1002235)

To a solution of tert-butyl 5-[[3-(isobutylamino)-N-methyl-anilino]methyl]imidazole-1-carboxylate (42 mg, 0.12 mmol) in NMP (1 ml) was added DIEA (0.06 ml, 0.35 mmol). The solution was cooled to 0° C. and butanoyl chloride (18.73 mg, 0.18 mmol) added. The reaction was allowed to warm to room temperature and stirred for 90 minutes (LCMS). The reaction mixture was diluted with DCM (5 ml) and washed with NaOH (2M aq., 4 ml). Organics were filtered through a hydrophobic frit and the mixture concentrated to afford an NMP solution of crude product. The solution was diluted with HCl (4M diox., 2 ml) and the mixture stirred for 2 hours and concentrated to remove dioxane and excess HCl. Sodium carbonate was added to the resulting NMP solution and the mixture stirred for 30 minutes and then filtered. Purification by prep. HPLC (basic) followed by evaporation of solvent from the appropriate fractions and drying afforded product as a gum, 18 mg, ¹H NMR indicates product is not sufficiently pure for submission for testing. Purification by prep. HPLC (basic, late focussed gradient) followed by evaporation of solvent from the appropriate fractions and drying under vacuum product, 15 mg (39%) afforded of N-[3-[1H-imidazol-5-ylmethyl(methyl)amino]phenyl]-N-isobutyl-butanamide.

### Procedure 18 (ESC1002236)

tert-butyl 5-[[7-(isobutylamino)-3,4-dihydro-2H-quinolin-1-yl]methyl]imidazole-1-carboxylate (50%, 79 mg, 0.1 mmol) was dissolved in DCM (1 ml) and DIEA (0.18 ml, 1.03 mmol) was added. The solution was cooled to 0° C. and 2-fluorobenzoyl chloride (0.02 ml, 0.15 mmol) was added. The reaction was allowed to warm to room temperature and stirred for 3 hours (LCMS). The reaction mixture was diluted with DCM (5 ml) and washed with water (2 ml). Organics were filtered through a hydrophobic frit and solvent was evaporated to afford crude intermediate. Purification by prep. HPLC (basic) followed by evaporation of solvent from the appropriate fractions afforded product. ¹H NMR/LCMS indicates product is not sufficiently pure for submission for testing. Purification by flash chromatography (silica column, DCM 0% to 75% gradient of a solution of DCM:MeOH:NH₄OH 90:10:1) followed by evaporation of solvent from the appropriate fractions and drying under vacuum afforded product, 10 mg (23.9%) of 2-fluoro-N-[1-(1H-imidazol-5-ylmethyl)-3,4-dihydro-2H-quinolin-7-yl]-N-isobutyl-benzamide.

### Procedure 19 (ESC1002266)

2-fluoro-N-(2-methoxyethyl)-N-[3-(methylamino)phenyl]benzamide (200 mg, 0.66 mmol) and 1H-imidazole-5-carbaldehyde (76.27 mg, 0.79 mmol) were suspended in DCM (2 ml) and acetic acid (0.06 ml, 0.99 mmol) was added. The mixture was stirred for 20 minutes then sodium triacetoxyborohydride (280.39 mg, 1.32 mmol) was added and stirring continued at room temperature for 18 hours (LCMS). The reaction mixture was diluted with DCM (30 ml) and washed with NaOH (1M aq., 10 ml). The aqueous wash was extracted with DCM (10 ml) then organics were combined, washed with brine, dried over sodium sulphate, filtered and solvent was evaporated under reduced pressure to afford crude product, 362 mg. Purification by flash chromatography (silica column, DCM 0% to 100% gradient of a solution of DCM:MeOH:NH₄OH 100:10:1) followed by evaporation of solvent from the appropriate fractions afforded product 0500A as a colourless gum. Not sufficiently pure for submission for testing. Purification by prep. HPLC (basic) followed by evaporation of solvent from the appropriate fractions afforded product 0500B. Analysis confirms product is pure for testing but contains residual MeOH. Further drying under vacuum afforded product as a colourless gum, 180 mg (71.2%) of 2-fluoro-N-[3-[1H-imidazol-4-ylmethyl(methyl)amino]phenyl]-N-(2-methoxyethyl)benzamide.

### ESC1002267

Procedure 19 with 2-fluoro-N-(2-isopropoxyethyl)-N-[3-(methylamino)phenyl]benzamide (151 mg, 0.46 mmol), 1H-imidazole-5-carbaldehyde (52.7 mg, 0.55 mmol), acetic acid (0.04 ml, 0.69 mmol) and sodium triacetoxyborohydride (193.72 mg, 0.91 mmol).

### Procedure 20 (ESC1002314 to ESC1002327, ESC1002337 to ESC1002341 and ESC1002422 to ESC1002424)

Suzuki Coupling: To a microwave vial was added the aryl bromide, the desired boronic acid the solvents (dioxane+water) and the mixture was bubbled with argon for ˜5 min. Then the cat mix first choice was added to the reaction mixture and the vessel was sealed before heating it to 80° C. overnight. The vial was let to cool to rt then, plugged on silica gel eluting it with EtOAc. The solvents were removed via a Genevac then the mixture was purified by HPLC (acidic). The desired fraction were collected and re-purified if necessary. The mixtures were further dried overnight in a Vac oven (4 mbar, at 50° C.)

ESC1002420 and ESC1002421 were obtained by SFC chiral separation of ESC1002338.

ESC1002766 and ESC1002767 were obtained by SFC chiral separation of ESC1002324.

ESC1002768 and ESC1002769 were obtained by SFC chiral separation of ESC1002321.

### Procedure 21 (ESC1002334)

N-[2-(dimethylamino)ethyl]-2-fluoro-N-[3-(methylamino)phenyl]benzamide (35 mg, 0.11 mmol) and 1H-imidazole-5-carbaldehyde (12.8 mg, 0.13 mmol) were combined in DCM (2 ml) and acetic acid (0.01 ml, 0.17 mmol) was added. The mixture was stirred at room temperature for 20 minutes then sodium triacetoxyborohydride (47.04 mg, 0.22 mmol) was added and stirring was continued for 16 hours (LCMS). Additional 1H-imidazole-5-carbaldehyde (5.33 mg, 0.06 mmol) was added and stirring continued for 24 hours (LCMS). The reaction mixture was diluted with DCM (15 ml) and washed with NaOH (1M aq., 10 ml). The aqueous wash was extracted with DCM (10 ml) then organics were combined, washed with brine, dried over sodium sulphate, filtered and solvent was evaporated under reduced pressure to afford crude product, 42 mg. Purification by prep. HPLC (basic, late focussed gradient) followed by evaporation of solvent from the appropriate fractions and drying under vacuum afforded product as a colourless gum, 20 mg (45.6%) of N-[2-(dimethylamino)ethyl]-2-fluoro-N-[3-[1H-imidazol-4-ylmethyl(methyl)amino]phenyl]benzamide.

### Procedure 22 (ESC1002344)

N-(cyclopropylmethyl)-2-fluoro-N-[3-[methyl-[[1-(2-trimethylsilylethoxymethyl)imidazol-4-yl]methyl]amino]phenyl]benzamide (20 mg, 0.04 mmol) was dissolved in MeOH (0.25 ml) and THF (0.25 ml) and HCl (5M aq., 0.2 ml) was added. The reaction was heated to reflux for 24 hours. The reaction mixture was concentrated under reduced pressure and the resulting residue dissolved in DCM and washed with saturated sodium bicarbonate solution. Organics were filtered through a hydrophobic frit and solvent was evaporated under reduced pressure to afford crude product. Purification by prep. HPLC (basic, late focussed gradient method) afforded two major products (largest peak due to boronate complex impurity brought through from previous reaction, structure as shown). Evaporation of solvent from the appropriate fractions and drying afforded desired product, 3.2 mg (21.5%) of N-(cyclopropylmethyl)-2-fluoro-N-[3-[1H-imidazol-4-ylmethyl(methyl)amino]phenyl]benzamide.

### Procedure 23 (ESC1002344)

To a flask were added 2-fluoro-N-isobutyl-N-[3-(methylamino)phenyl]benzamide (100 mg, 0.33 mmol), 2-(3-bromopropyl) isoindoline-1,3-dione (100 mg, 0.37 mmol), K₂CO₃ (138.03 mg, 1 mmol) and diluted in acetonitrile (15 ml). The mixture was refluxed for 5 days. A portion of NaH in mineral oil was added and the mixture was stirred at rt for 6 h. The mixture was quenched with water and extracted with EtOAc (3×25 ml). The organic layers were dried over Na₂SO₄ and reduced before being charged on a 10 g silica cartridge and eluted with Hep:EtOAc (0-50%). Although separation occurred it wasn't clean therefore the mixture was purified on HPLC (acidic). The desired fractions were collected and reduced to obtain 10 g of the desired compound. Due to the greasy character it was decided to salt the compound by diluting it in 2 ml of Ether and adding 0.1 ml of 3M HCl in dioxane. The mixture was reduced to form a clear white solid.

### Procedure 24 (ESC1002399)

N-(cyclopropylmethyl)-4-methyl-N-[3-[methyl-[[1-(2-trimethylsilylethoxymethyl)imidazol-4-yl]methyl]amino]phenyl]benzamide (27 mg, 0.05 mmol) was dissolved in MeOH (1 ml) and THF (1 ml) and 5M HCl (1 ml) was added. The reaction was heated in a microwave to 150° C. for 30 minutes. The reaction mixture was concentrated under reduced pressure and the resulting brown oil partitioned between DCM and saturated sodium bicarbonate solution. Organics were filtered through a hydrophobic frit and solvent was evaporated under reduced pressure to afford crude product (18 mg). Purification by prep. HPLC (basic, late focussed gradient) followed by evaporation of solvent from the appropriate fractions afforded product as a pale yellow gum, 2.6 mg (13%) of N-(cyclopropylmethyl)-N-[3-[1H-imidazol-4-ylmethyl(methyl)amino]phenyl]-4-methyl-benzamide.

### Procedure 25 (ESC1002125 to ESC1002437, ESC1002624 to ESC1002627, ESC1002661 to ESC1002666)

Amide coupling: to a vial were added 2-[2-(3-methoxyphenyl)phenyl]pyrrolidine (50.67 mg, 0.2 mmol), the acid, dichloromethane (4 ml), DIEA (0.09 ml, 0.5 mmol) and O-(7-Azabenzotriazol-1-yl)-N,N,N′,N′-tetramethyluronium hexafluorophosphate (114.07 mg, 0.3 mmol) then the mixture was stirred at rt overnight. The solvents were removed (Genevac) then purified on HPLC (prep acidic). The desired fractions were collected and dried (Genevac). The products were transferred to a vial then dried over Vac oven (50° C., 3 mbar) for 72 h. NB: Most compounds present a very blurry ¹H NMR spectra due to rotameric effects. A high temp NMR was therefore carried out. Some compounds presented clear spectra at rt as a mixture of diastereoisomers, but upon heating became blurry.

ESC1002754 and ESC1002755 were obtained by SFC chiral separation of ESC1002666.

ESC1002756 and ESC1002757 were obtained by SFC chiral separation of ESC1002626.

### Procedure 26 (ESC1002454)

2,4-difluoro-N-isobutyl-N-[3-[methyl-[[1-(2-trimethylsilylethoxymethyl)imidazol-4-yl]methyl]amino]phenyl]benzamide (37 mg, 0.07 mmol) was dissolved in MeOH (1 ml) and 5M HCl (0.5 ml) added. The reaction was heated to reflux for 6 hours then stirred at room temperature for 16 hours, overnight. Heating was continued for 5 hours (LCMS) then the reaction mixture was concentrated under reduced pressure. The crude HCl salt was dissolved in DCM (2 ml) and washed with saturated sodium bicarbonate solution (2 ml). The aqueous wash was re-extracted with DCM (2 ml) then organics were combined, filtered through a hydrophobic frit and solvent was evaporated under reduced pressure to afford crude product, 27 mg. Purification by prep. HPLC (basic, late focussed gradient method) followed by evaporation of solvent from the appropriate fractions and drying afforded product, 15 mg (53.8%) of 2,4-difluoro-N-[3-[1H-imidazol-4-ylmethyl(methyl)amino]phenyl]-N-isobutyl-benzamide.

### ESC1002455

Procedure 26 with 2-fluoro-N-isobutyl-N-[3-[methyl-[[1-(2-trimethylsilylethoxymethyl)imidazol-4-yl]methyl]amino]phenyl]-4-(t rifluoromethyl)benzamide (73%, 43 mg, 0.05 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 13 mg (53.4%) of 2-fluoro-N-[3-[1H-imidazol-4-ylmethyl(methyl)amino]phenyl]-N-isobutyl-4-(trifluoromethyl)benzamide.

### ESC1002456

Procedure 26 with 2,6-difluoro-N-isobutyl-N-[3-[methyl-[[1-(2-trimethylsilylethoxymethyl)imidazol-4-yl]methyl]amino]phenyl]benzamide (25 mg, 0.05 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 8 mg (42.5%) of 2,6-difluoro-N-[3-[1H-imidazol-4-ylmethyl(methyl)amino]phenyl]-N-isobutyl-benzamide.

### ESC1002457

Procedure 26 with 2-chloro-N-isobutyl-N-[3-[methyl-[[1-(2-trimethylsilylethoxymethyl)imidazol-4-yl]methyl]amino]phenyl]benzamide (86%, 33 mg, 0.05 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 9 mg (42.1%) of 2-chloro-N-[3-[1H-imidazol-4-ylmethyl(methyl)amino]phenyl]-N-isobutyl-benzamide.

### ESC1002459

Procedure 26 with 4-ethyl-N-isobutyl-N-[3-[methyl-[[1-(2-trimethylsilylethoxymethyl)imidazol-4-yl]methyl]amino]phenyl]benzamide (83%, 34 mg, 0.05 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 13.5 mg (63.8%) of 4-ethyl-N-[3-[1H-imidazol-4-ylmethyl(methyl)amino]phenyl]-N-isobutyl-benzamide.

### ESC1002460

Procedure 26 with 5-chloro-N-isobutyl-N-[3-[methyl-[[1-(2-trimethylsilylethoxymethyl)imidazol-4-yl]methyl]amino]phenyl]thiophene-2-carboxamide (82%, 35 mg, 0.05 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 16.2 mg (74.7%) of 5-chloro-N-[3-[1H-imidazol-4-ylmethyl(methyl)amino]phenyl]-N-isobutyl-thiophene-2-carboxamide.

### Procedure 27 (ESC1002462 and ESC1002698 to ESC1002702 and ESC1002704)

To a microwave vial were added [2-(2-bromophenyl)pyrrolidin-1-yl]-(3-methyl-2-thienyl)methanone (80 mg, 0.23 mmol) (or generally the desired aryl bromide), phenylboronic acid (55.7 mg, 0.46 mmol) (or generally the desired boronic acid) the solvents (dioxane+water) then the mixture was bubbled with argon for ˜5 min. Catkit first choice was added to the reaction mixture and the vessel was sealed before heating it to 80° C. overnight. Once completed the mixture was diluted with EtOAc and water and extracted with EtOAc (3×15 ml). The org. layers are dried over Na₂SO₄, and reduced before charging the mixture on a 10 g silica cartridge. The mixture was purified on an Isolera and the desired fractions were collected. The solids obtained were further dried in a vac. oven (at 50° C. over night). The compounds was analysed by ¹H NMR at 373K to resolve rotameric effects.

### Procedure 28 (ESC1002463)

To a microwave vial were added (3-methyl-2-thienyl)-[2-[2-(1H-pyrazol-4-yl)phenyl]pyrrolidin-1-yl]methanone (32 mg, 0.09 mmol), 2-bromoethanol (8.07 μl, 0.11 mmol), K₂CO₃ (19.66 mg, 0.14 mmol) and acetonitrile (2 ml). The vessel was sealed and the subjected to microwave radiation for 20 min at 150° C. 0.1 ml of 2-bromoethanol was added and the mixture was subjected to 150° C. for 30 min. The mixture was subjected to 45 min then 3 h at 150° C. The mixture was filtered reduced and purified by HPLC (basic) the desired fraction were collected and reduced. The compound was further dried over Genevac (high bp) and vac oven (3 mbar at 52° C. overnight). The compounds was analysed by ¹H NMR at 373K to resolve rotameric effects.

### Procedure 29 (ESC 1002164 to ESC1002466)

Suzuki cross coupling method. To a vial microwave vial were added the aryl bromide, the desired boronic acid the solvents (dioxane+water) and then the mixture was bubbled with argon for ˜5 min. Cat mix first choice was added to the reaction mixture and the vessel was sealed before heating it to 80° C. for 16 h. The mixture was allowed to cool to rt then diluted with EtOAc and Water and extracted with EtOAc (3×15 ml). The org. layers were filtered through celite, dried over Na2SO4, reduced. To the crude intermediate was added TFA (17.1 mg, 0.15 mmol) and Dichloromethane (2 ml) and the mixture was stirred at rt for 3 h. The mixtures were reduced (Genevac) then purified by HPLC prep. acidic (except reaction 4). the desired fractions were collected and reduced before flushing them through a 10 g silica cartridge on an Isolera using Hep:EtOAc (0-100%). the desired fractions were collected, reduced and dried over Geneva and vac oven (3 mbar at 52° C.).

ESC1002789 and ESC1002790 were obtained by SFC chiral separation of ESC1002320.

### ESC1002491

Procedure 26 with N-isobutyl-2-methyl-N-[3-[methyl-[[1-(2-trimethylsilylethoxymethyl)imidazol-4-yl]methyl]amino]phenyl]benzamide (19 mg, 0.04 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 6.1 mg (43.2%) of N-[3-[1H-imidazol-4-ylmethyl(methyl)amino]phenyl]-N-isobutyl-2-methyl-benzamide.

### ESC1002492

Procedure 26 with N-isobutyl-2,4-dimethyl-N-[3-[methyl-[[1-(2-trimethylsilylethoxymethyl)imidazol-4-yl]methyl]amino]phenyl]benzamide (13 mg, 0.02 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 3.9 mg (40%) of N-[3-[1H-imidazol-4-ylmethyl(methyl)amino]phenyl]-N-isobutyl-2,4-dimethyl-benzamide.

### ESC1002493

Procedure 26 with N-isobutyl-N-[3-[methyl-[[1-(2-trimethylsilylethoxymethyl)imidazol-4-yl]methyl]amino]phenyl]-2-(trifluorom ethyl)benzamide (24 mg, 0.04 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 4.6 mg (25%) of N-[3-[1H-imidazol 4-ylmethyl(methyl)amino]phenyl]-N-isobutyl-2-(trifluoromethyl)benzamide.

### Procedure 30 (ESC1002504 to ESC1002506 and ESC1002597)

Amine and the desired halogen was added to a flask, and the mixture was diluted in tetrahydrofuran (2 ml) and TEA (0.07 ml, 0.5 mmol) and the mixture was heated to 70° C. overnight. LCMS revealed >50% conversion. The mixture was diluted in EtOAc and sat NaHCO₃ solution then extracted with EtOAc (3*3 ml). The org. phases were dried over Na₂SO₄ and reduced before being charged on a 10 g silica cartridge and purified on an Isolera (Hep:EtOAc 0-50%). The purification was repeated. The compound was further purified on HPLC and the desired fractions were dried. The paste was slated using 0.1 ml of 4M HCl in Dioxane, and the compound was dried before submission (Genevac and vac oven 3 mbar at 52° C.).

### Procedure 31 (ESC1002507)

N-[3-[3-aminopropyl(methyl)amino]phenyl]-2-fluoro-N-isobutyl-benzamide (40 mg, 0.11 mmol) was added to a flask, Acetic anhydride (0.02 ml, 0.25 mmol) and Pyridine (2 ml) as solvent. The mixture was stirred at rt for 3 h. The mixture was reduced and purified by a prep. HPLC, under basic conditions. The obtained compound was salted using HCl in dioxane (0.1 ml) and further reduced.

### ESC1002508

Procedure 26 with N-butyl-2-fluoro-N-[3-[methyl-[[1-(2-trimethylsilylethoxymethyl)imidazol-4-yl]methyl]amino]phenyl]benzamide (64 mg, 0.13 mmol) in place of 2,4-difluoro-N-isobutyl-N-[3-[methyl-[[1-(2-trimethylsilylethoxymethyl)imidazol-4-yl]methyl]amino]phenyl]benzamide. The product was obtained as 18 mg (37.8%) of N-butyl-2-fluoro-N-[3-[1H-imidazol-4-ylmethyl(methyl)amino]phenyl]benzamide.

### ESC1002516

Procedure 26 with 2-fluoro-N-[(5-methylisoxazol-3-yl)methyl]-N-[3-[methyl-[[1-(2-trimethylsilylethoxymethyl)imidazol-4-yl]methyl]amino]phenyl]benzamide (20 mg, 0.04 mmol) in place of 2,4-difluoro-N-isobutyl-N-[3-[methyl-[[1-(2-trimethylsilylethoxymethyl)imidazol-4-yl]methyl]amino]phenyl]benzamide. Purification by prep. HPLC (acidic method) and SCX, product eluted with 2M methanolic ammonia followed by evaporation of solvent and drying afforded product as 5 mg (32.8%) of 2-fluoro-N-[3-[1H-imidazol-4-ylmethyl(methyl)amino]phenyl]-N-[(5-methylisoxazol-3-yl)methyl]benzamide. Trace MeOH impurity but sufficiently pure for submission for testing.

### ESC1002517

Procedure 26 with 2 N-(2,2-dimethylpropyl)-2-fluoro-N-[3-[methyl-[[1-(2-trimethylsilylethoxymethyl)imidazol-4-yl]methyl]amino]phenyl]benzamide (26 mg, 0.05 in place mmol) of 2,4-difluoro-N-isobutyl-N-[3-[methyl-[[1-(2-trimethylsilylethoxymethyl)imidazol-4-yl]methyl]amino]phenyl]benzamide. Product obtained as 11.4 mg (58.3%) of N-(2,2-dimethylpropyl)-2-fluoro-N-[3-[1H-imidazol-4-ylmethyl(methyl)amino]phenyl]benzamide.

### Procedure 32 (ESC1002575)

2-fluoro-N-isobutyl-4-methyl-N-[3-[methyl-[[1-(2-trimethylsilylethoxymethyl)imidazol-4-yl]methyl]amino]phenyl]benzamide (39 mg, 0.07 mmol) was dissolved in MeOH (1 ml) and 5M HCl (0.5 ml) added. The solution was heated to 70° C. for 18 hours. The reaction mixture was basified with addition of NaOH (2M aq.) and extracted with DCM (2×25 ml). Organics were combined, dried over sodium sulphate, filtered and solvent was evaporated under reduced pressure to afford crude product, 30 mg. LCMS indicated recovered starting material (suggests HCl also evaporated during reaction). The recovered material was dissolved in MeOH (1 ml) and HCl (5M, 0.5 ml) added. The reaction was heated to 70° C. for 18 hours (LCMS) then concentrated in a genevac. The resulting residue was dissolved in DCM (2 ml) and washed with saturated sodium bicarbonate solution (2 ml). The wash was re-extracted with DCM (2 ml) then organics were combined, filtered through a hydrophobic frit and solvent was evaporated in a genevac. The crude residue was purified by prep. HPLC (basic, late focussed gradient) followed by evaporation of solvent and drying to afford product, 11 mg (37.5%) of 2-fluoro-N-[3-[1H-imidazol-4-ylmethyl(methyl)amino]phenyl]-N-isobutyl-4-methyl-benzam

### ESC1002614

Procedure 26 with N-isobutyl-N-[3-[methyl-[[1-(2-trimethylsilylethoxymethyl)imidazol-4-yl]methyl]amino]phenyl]-2-oxo-1H-pyridine-3-carboxamide (7 mg, 0.01 mmol) in place of 2,4-difluoro-N-isobutyl-N-[3-[methyl-[[1-(2-trimethylsilylethoxymethyl)imidazol-4-yl]methyl]amino]phenyl]benzamide. Product obtained as 1.2 mg (23%) of N-[3-[1H-imidazol-4-ylmethyl(methyl)amino]phenyl]-N-isobutyl-2-oxo-1H-pyridine-3-carboxamide.

### ESC1002624

Procedure 26 with N-isobutyl-2-methylsulfonyl-N-[3-[methyl-[[1-(2-trimethylsilylethoxymethyl)imidazol-4-yl]methyl]amino]phenyl]benzamide (60 mg, 0.11 mmol) in place of 2,4-difluoro-N-isobutyl-N-[3-[methyl-[[1-(2-trimethylsilylethoxymethyl)imidazol-4-yl]methyl]amino]phenyl]benzamide. Product obtained as 29 mg (62.6%) of N-[3-[1H-imidazol-4 ylmethyl(methyl)amino]phenyl]-N-isobutyl-2-methylsulfonyl-benzamide.

### Procedure 33 (ESC1002656)

N-isobutyl-N-[3-[methyl-[[1-(2-trimethylsilylethoxymethyl)imidazol-4-yl]methyl]amino]phenyl]pyrazine-2-carboxamide (54 mg, 0.11 mmol) was dissolved in TBAF (1M in THF, 2.5 ml) and the reaction heated to 120° C. in a microwave for 60 minutes (LCMS). The reaction mixture was concentrated under reduced pressure and the afforded residue partitioned between DCM (10 ml) and 10% citric acid solution (10 ml). The acid solution was separated (organics discarded) and made basic with addition of NaOH (2M aq.) then extracted with DCM (2×10 ml). Organics were combined, filtered through a hydrophobic frit and solvent was evaporated under reduced pressure to afford crude product, 95 mg. Purification by prep. HPLC afforded the desired product. The product was purified by flash chromatography (silica column, elution with isocratic 90:10:1 DCM:MeOH:NH₄OH). Evaporation of solvent from the appropriate fractions and drying afforded desired product as a pale yellow solid, 18 mg (45.2%) of N-[3-[1H-imidazol-4-ylmethyl(methyl)amino]phenyl]-N-isobutyl-pyrazine-2-carboxamide.

### Procedure 34 (ESC1002696 and ESC1002697)

To a solution of Reactant 0 (37 mg, 0.1 mmol) in 2-MeTHF was added 1M (CH₃)₂S. BH₃ in 2-MeTHF (0.29 ml) and the mixture was heated to reflux. After 16 h the mixture was analysed by LCMS which indicated ˜60% conversion. More 1M (CH₃)₂S. BH₃ in 2-MeTHF (0.5 ml) was added and the mixture was refluxed for a further 5 h. The solution was allowed to cool to rt and quenched with 25 ml of 1M HCl solution. The crude was then extracted with EtOAc (3×20 ml), dried over Na₂SO₄ and reduced. The crude was first purified on prep HPCL (acidic conditions). With the desired fraction dried over a Genevac, the compound was charged on a 10 g silica cartridge and purified on an Isolera, using DCM:MeOH (0-10%) as eluant. The compound was then intensively dried on a Genevac then a Vac Oven (50° C., 3bar, >16 h).

### Procedure 35 (ESC1002696)

Reactant 1 (73 mg, 0.21 mmol), Reactant 2 (43.64 mg, 0.27 mmol), dichloromethane (5 ml), DIEA (0.09 ml, 0.52 mmol) and O-(7-Azabenzotriazol-1-yl)-N,N,N′,N′-tetramethyluronium hexafluorophosphate (117.75 mg, 0.31 mmol) were added to a vial then the mixture was stirred at rt overnight. The mixture was partitioned with water and DCM and further extracted with DCM. The org phase was dried over a phase separator and reduced. The mixture was charged on a 10 g silica cartridge and purified on an Isolera (Hep:EtOAc 0-40%). The desired fractions were collected dried and set to the following step. Product 1 (68 mg, 0.14 mmol) was diluted with Acetonitrile (9.5 ml) and water (0.5 ml), and the mixture was homogenised at rt. Then DBU (0.02 ml, 0.14 mmol) and the mixture was stirred at rt for 1 h. DBU (0.1 ml) was added and the mixture was stirred for a further 2 h. The mixture was partitioned between water and EtOAc and further extracted with EtOAc. The organic layers were dried over Na₂SO₄ then the mixture was reduced and purified on a prep HPLC (Acidic). The desired fractions were dried on a Genevac and charged on a 10 g silica cartridge and further purified on an Isolera using Hep:EtOAc (0-100%) as eluant. The desired fractions were collected and extensively dried over Genevac and Vac Oven (50° C., 3 mbar, >16 h).

### Procedure 36 (ESC1002762)

To a solution of the appropriate N-Methylaniline analogue in toluene is added Cl—CH₂CN followed by K₂CO₃. The mixture is heated at reflux overnight. After cooling to RT The mixture is diluted with DCM and filtered. The filter residue is washed with DCM and the combined filtrates are evap. i.vac. The residual material is repurified on a 5 g Si-SPE column: Rf=0.2 in heptane/MeCO₂Et=2/1 (unreacted starting material elutes slightly before with an Rf=0.3). Final purification is carried out on basic prepHPLC where the desired product elutes between 8-8.4 min.

### Procedure 37 (ESC1002764)

2-fluoro-{N}-[3-(1,2,4-triazol-4-yl)phenyl]benzamide (90.0 mg, 0.319 mmol) was dissolved in DMF (1 ml). Cs₂CO₃ (0.312 g, 0.957 mmol), NaI (0.00956 g, 0.0638 mmol) and 1-bromo-2-methyl-propane (0.104 ml) 0.957 mmol) were added to a vial. The vial was sealed and heated to 80° C. for 3 hours. LCMS indicated that product was forming. Further 1-bromo-2-methyl-propane (0.208 ml) was added and the reaction heated for a further 3 hours. The reaction was diluted with ethyl acetate and water. The organic was washed with water, brine, dried over Na2SO4 and the solvent removed at reduced pressure. The resulting residue was purified by flash chromatography (5 g Biotage zip) eluting with a gradient 0-5% methanol in DCM. The appropriate fractions were combined, and the solvent removed at reduced pressure to afford product which was found to be impure. Product was further purified by acidic prep HPLC. The appropriate fractions were combined and the solvent removed in a Genevac to afford recovered starting amide and desired product.

### Procedure 38 (ESC1002765)

2-fluoro-N-(3-imidazol-1-ylphenyl)benzamide (60 mg, 0.21 mmol) was stirred in DMF (1 mL). NaH disp. in mineral oil (60%, 12.8 mg, 0.32 mmol) was added. The reaction was placed under an argon atmosphere and stirred for 10 minutes. 1-bromo-2-methyl-propane (46.39 μl, 0.43 mmol) was added and the reaction heated to 60° C. for 1 hour. Further 1-bromo-2-methyl-propane (46.39 μl, 0.43 mmol) was added and the reaction heated for a further 30 minutes. The reaction was diluted with ethyl acetate and quenched with water. The organic layer washed with water (×3), brine, dried over Na₂SO₄ and concentrated at reduced pressure. The resulting residue was purified by flash chromatography (5 g Biotage SiO2 zip) eluting with a gradient 0-95:5:05 DCM:MeOH:NH₄OH. The appropriate fractions were combined and the solvent removed at reduced pressure to afford product.

### Procedure 39 (ESC1002775)

Suzuki: (2-(2-bromophenyl)pyrrolidin-1-yl)(3-chlorothiophen-2-yl)methanone (0.1 g, 0.27 mmol) and (4-fluoro-3-hydroxyphenyl) boronic acid (63.09 mg, 0.4 mmol) were suspended in MeTHF (1 mL). First choice catkit (Pd₂(dba)₃, PCy₃, K₃PO₄ (135.96 mg, 0.27 mmol) was added to the reaction mixture which was heated to 150° C. for 20 min. The reaction mixture was filtered through celite which was washed with EtOAc. The EtOAc was washed with water, dried (Na₂SO₄), filtered and concentrated in vacuo. The resulting material was purified by HPLC (acidic prep.). The material was further purified by HPLC (base prep.). The relevant fractions were collected and concentrated in vacuo, and dried in vacuo for 18 hours at 50° C. giving the desired compound as a white solid (28 mg, 26%). NMR experiments indicated that the material contains conformational isomers.

ESC1002838 and ESC1002839 were obtained by SFC chiral separation of ESC1002775.

### ESC1002776

Procedure 39 with (3-fluoro-5-hydroxyphenyl) boronic acid in place of (4-fluoro-3-hydroxyphenyl) boronic acid. The reaction mixture was irradiated to 150° C. for 20 min. The reaction mixture was filtered through celite which was washed with EtOAc. The EtOAc was washed with water, dried (Na₂SO₄), filtered and concentrated in vacuo. The resulting material was purified by HPLC (acidic prep). The material was further purified by HPLC (base prep.). The relevant fractions were collected and concentrate in vacuo, and dried for 18 h at 50° C. in vacuo giving the desired compound (11.7 mg, 11%). NMR studies indicated that the product existed as a mixture of rotamers. High temperature NMR studies were conducted to confirm this.

### ESC1002800 and ESC102801

Procedure 27. Once heating was completed, the mixture was plugged on silica (˜1 g), and eluted with EtOAc. Then the organic phases were reduced and charged on a 10 g silica cartridge and purified on an Isolera using Hep:EtOAc (0-50%) as eluant. The desired fractions were collected and reduced to afford the product in 72% yield. 30 mg was kept and the remaining 48 mg were separated. The compound was separated via chiral SFC. On an Analytical Ad-H column 4.6×250 mm: flow rate 5 ml/min-injection volume 20 microL-Co solvent Methanol 14%.

### Procedure 40 (ESC1002802)

2-fluoro-N-isobutyl-N-(5-(methylamino)pyridin-3-yl)benzamide (5 mg, 0.02 mmol) and 4-(chloromethyl)-4H-imidazole (0.01 g, 0.03 mmol) were dissolved in MeTHF. DIPEA (0.02 ml, 0.15 mmol) was added and the reaction mixture was allowed to stir at room temperature for 1 hour. The reaction mixture was heated to 70° C. and allowed to stir for three hours. MeCN (2 mL) was added to the reaction mixture which was allowed to stir at room temp for 2 hours. The reaction mixture was partitioned between water and EtOAc and the phases were separated. The aqueous layer was extracted with EtOAc and the combined organic layers were dried (Na₂SO₄), filtered and concentrated in vacuo giving 30 mg of residue. The mixture was purified by HPLC (acidic prep, rT=5.22 min), the relevant fractions were collected and concentrated in vacuo giving <5 mg of product. The aqueous layer (pH 6-7) was basified to pH 9 with 2M NaOH (aq.) and extracted with EtOAc. The aqueous layer was further basified to pH 14 and extracted with EtOAc. The aqueous layer was concentrated in vacuo giving a solid residue. This was extracted with MeOH, filtered and concentrated in vacuo. The resulting residue was purified by HPLC (acidic prep, rT=5.22 min), and the relevant fractions were concentrated in vacuo. This material was combined with the material from earlier HPLC runs, the combined material was dried at 50° C. for 18 h in vacuo giving a white solid (17 mg, 21%). NMR experiments indicated that this material exists as rotamers. High temperature NMR experiments confirmed that the structure was correct.

### Procedure 41 (ESC1002804)

(R)-2-(3′-methoxy-[1,1′-biphenyl]-2-yl)pyrrolidine (330 mg, 1.3 mmol) and 3-chlorothiophene-2-carboxylic acid (0.42 g, 2.61 mmol) were dissolved in MeTHF (5 mL). DIPEA (0.53 ml, 3.91 mmol) and HATU (0.49 g, 1.3 mmol) were added to the reaction mixture which was allowed to stir at room temperature for 18 hours. The reaction mixture was diluted with water and EtOAc and the phases were separated. The aqueous phase was extracted with EtOAc (×2) and the combined organic layer was dried (Na₂SO₄), filtered and concentrated in vacuo onto silica gel. The material was purified by flash column chromatography (0-40% EtOAc in n-heptanes). Giving material with minor impurities (270 mg, 52%). An aliquot of material was purified further by HPLC (basic prep, rT=9.33 min), the relevant fractions were collected and concentrated in vacuo. The material was dried at 50° C. in vacuo for 18 h giving the desired compound as a white solid.

### ESC1002817

Procedure 37 with N-isobutyl-4-methyl-N-[4-(methylamino)pyrimidin-2-yl]benzamide (25 mg, 0.08 mmol), 4-(chloromethyl)-1H-imidazole; hydrochloride (25.64 mg, 0.17 mmol), Cs₂CO₃ (40.95 mg, 0.13 mmol) and NaI (0.31 mg, 0 mmol). Heating was continued over the weekend. An additional 1.5 eq of base was added and heating continued. Additional base (3 eq) and chloride (2 eq) were added and heating continued overnight. The crude mix was filtered and purified on basic HPLC and the fraction corresponding to the product (by LCMS) was collected and concentrated in the genevac. NMR and LCMS suggest material is consistent with desired product and of sufficient purity for testing (though small baseline impurities are visible in the NMR). ˜5 mg obtained.

### ESC1002840 and ESC1002841

Procedure 39 with 2-fluoro-3-hydroxyphenyl) boronic acid (46 mg, 0.29 mmol) in place of (4-fluoro-3-hydroxyphenyl) boronic acid. (1 mL). The resulting material was purified by flash column chromatography (0-30% EtOAc in n-heptanes) giving the desired compound as a mixture of enantiomers (35 mg, 32%). The material was resolved by SFC (OD-H, 10 mm×250 mm) giving two enantiomers (peak 1, rT=4.52 min, ESC1002840-01) (peak 2, rT=5.36 min, ESC1002841-01). NMR indicates that the compounds have rotational isomers. High temperature NMRs indicate that the compounds are correct.

### Protein Expression and Purification for Crystallography

Human 17β-HSD10 (residues 1-261) was cloned into pNIC-CTHF vector with a TEV-cleavable C-terminal hexa-histidine tag and Flag tag. After transformation into E. coli (BL21(DE3) pRARE2), expression was performed in TB auto induction medium (FroMedium), supplemented with 20 g/L glycerol, anti-foam, 50 μg/mL kanamycin and 34 μg/mL chloramphenicol. Cultures were grown for four hours at 37° C., then the temperature was dropped to 18° C. and the cultures were grown for another 42 hours. Cells were spun at 4,000×g for 20 minutes, and after discarding the supernatant, frozen for 2 hours. After thawing, the cell pellets were resuspended in a buffer (10 mM HEPES, 5% Glycerol, 500 mM NaCl, 0.5 mM TCEP, pH 7.5) supplemented with 0.5 mg/mL lysozyme and 1 μg/mL benzonase, vortexed and incubated at room temperature for 30 mins. 2% Triton X-100 was added and the cells were frozen overnight at −80° C. On thawing, the cells were supplemented with 10 mM imidazole and stirred for 1 hour at room temperature. Cells were then centrifuged for one hour at 4,000×g and the supernatant applied to Hi GraviTrap column (GE healthcare) equilibrated with binding buffer (10 mM HEPES, 5% glycerol, 500 mM NaCl, 0.5 mM TCEP, pH 7.5). The column was washed twice with the binding buffer supplemented with 10 mM imidazole. ABAD was eluted with the binding buffer supplemented with 300 mM imidazole. The eluted protein was applied to a PD-10 desalting column (GE Healthcare) and eluted with binding buffer supplemented with 10 mM imidazole. The C-terminal affinity tag was removed by TEV cleavage overnight and uncleaved protein was removed by reverse IMAC by applying it again to a His GraviTrap column. The flow-through of ABAD was supplemented with 1/10^{th} the volume of 1M Arginine/1M Glutamine mix (pH 7.5) and was concentrated and purified further by size exclusion chromatography using a YARRA SEC-2000 PREP column (Phenomenex), equilibrated with binding buffer. Fractions containing ABAD were pooled, concentrated and stored at −80° C.

### Crystallisation

0.45 mM of 17β-HSD10 was incubated with 5 mM each of NADH and ESC1002033 compound at room temperature for 10 minutes. Sitting drop crystallization plate with 150 nL drop volume was setup with Hampton index screen (Hampton Research) at 20° C. Co-crystals were obtained in condition containing 5 mM magnesium chloride, 5 mM cobalt chloride, 5 mM nickel chloride, 5 mM cadmium chloride, 12% PEG3350 and 0.1M HEPES, pH 7.5. All crystals were harvested with 20% ethylene glycol as cryoprotectant and flash cooled in liquid nitrogen.

To obtain crystals with compound ESC1002332, a soaking out technique was used. 5 mM of compound ESC1002332 was soaked into a crystallization drop containing co-crystals with ESC1002033 for 10 mins. The crystals were then harvested as before.

### Synthesis of Compounds S1-Tn and S2-Tn

### LCMS Analytical Methods

### Analytical Method A (LCMS Monitoring)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 KV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (3.5 μm, 50×3 mm) with a flow rate of 1.20 ml/min. Two mobile phases were used, mobile phase A: 5 Mm NH₄CH₃CO₂ in water; mobile phase B: 5 Mm NH₄CH₃CO₂ in acetonitrile (ACN):water (90:10)], and they were employed to run a gradient conditions from 5% B for 0.75 minutes, from 5% to 30% in 1.25 minutes, and from 30% to 98% in 1.75 minutes, 98% B for 2.25 minutes and 5% B in 2.75 minutes and hold these conditions for 3.25 minutes in order to re-equilibrate the column (Total Run Time 3.25 minutes). An injection volume of 0.5 μL was used. The sample was monitored at 260 nm & 220 nm.

### Analytical Method B (LCMS Final)

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 seconds. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C8 column (1.7 μm, 50×2.1 mm) with a flow rate of 0.800 ml/minute. Two mobile phases were used, mobile phase A: 0.05% HCOOH in water; mobile phase B: 0.05% HCOOH in ACN:water (90:10)], and they were employed to run at gradient conditions from 5% B for 0.75 minutes, from 5% to 25% in 0.75 minutes, and from 25% to 95% in 1.50 minutes, 95% B for 1.00 minutes and 5% B in 0.50 minutes and hold these conditions for 0.60 minutes in order to re-equilibrate the column (Total Run Time 5.10 minutes). An injection volume of 0.5 μl was used. The sample was monitored at 260 nm & 220 nm.

### Preparative HPLC Method

Preparative HPLC was done on a Waters auto purification instrument. Column name: YMC-Actus Triat (250×20 mm, 5μ) operating at ambient temperature and flow rate of 16 mL/minute. Mobile phase: A=20 mM Ammonium bicarbonate in water, B=Acetonitrile; Gradient Profile: Mobile phase initial composition of 80% A and 20% B, then 70% A and 30% B in 3 minutes, then to 50% A and 50% B in 20 min., then to 5% A and 95% B in 21 minutes, held this composition up to 22 minutes. for column washing, then returned to initial composition in 23 minutes. and held till 25 minutes. UV detection e.g. 254 nM is used for the collection of fractions from HPLC. This description gives general method and variations in types of equipment, columns, mobile phase, detection wavelength, solvent gradient and run time also used to purify compounds.

### Preparative TLC Method

In preparative TLC, Silica gel 60 F254 analytical Prep thin layer chromatography (Prep-TLC) plates from Merck (Darmstadt, Germany) were used & crude material was run under a developed solvent system (30%-50% ethyl acetate hexane) separated are often applied as long streaks, rather than spots, in the sample application zone. After visualization under UV light, specific components were recovered by scraping the sorbent layer from the plate in the region of interest and eluting the separated material from the sorbent using a strong solvent (10% MeOH-DCM).

(S1 Series, DirectAmidation with (S)-2-Aryl Pyrrolidine and 5 & 6-Membered Het-Acids)

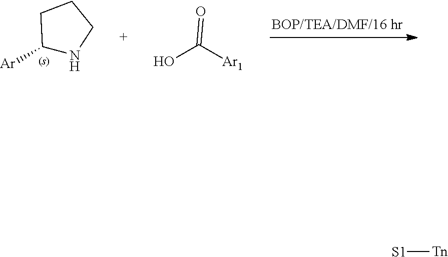

### General Procedure for Amidation Using BOP Reagent.

To a stirred solution of the acid (0.51 mmol, 1.0 eq) in dry dimethylformamide (DMF) (1.5 ml) at ice cooled condition, BOP reagent (0.51 mmol, 1.0 eq) was added portion wise then triethylamine (2.551 mmol, 5.0 eq) was added dropwise. Reaction mixture was stirred for few minutes at ice cold condition then(S)-aryl amine (0.51 mmol, 1.0 eq) in 0.5 ml DMF was added and reaction mixture was stirred at 23° C. for 16 h.

The reaction was monitored by TLC, and LCMS showed desired product obtained along with some impurities. The reaction mixture was quenched with saturated solution of NaHCO₃ and extracted with ethyl-acetate (2×25 ml). The combined organic layer was washed with brine, dried over anhydrous Na₂SO₄ and purified by prep-TLC (40% ethyl acetate in hexane) and lyophilized to afford desired compound.

For all final compounds high temperature NMR was recorded at 373K.

S1-T1: 4-methyl-5-{[(2S)-2-phenylpyrrolidin-1-yl]carbonyl}-1H-pyrazole (Off White solid, 26.95 mg, 20.69%)

¹H NMR (400 MHz, DMSO-d₆ at 373K) δ 12.54 (bs, 1H), 7.16-7.45 (m, 6H), 5.27 (bs, 1H), 3.84 (bs, 2H), 2.33 (bs, 1H), 2.04 (bs, 3H), 1.87 (bs, 3H)

LCMS Analytical Method B: rt=2.48 minutes, m/z 256.17 [M+H]⁺.

S1-T2: 2-{[(2S)-2-phenylpyrrolidin-1-yl]carbonyl}-1H-imidazole (White solid, 23.52 mg, 19.11%)

¹H NMR (400 MHz, DMSO-d₆ at 373K) δ 12.52 (bs, 1H), 7.18-7.27 (m, 7H), 5.28 (bs, 1H), 4.30 (bs, 2H), 2.33 (bs, 1H), 1.87 (bs, 3H).

LCMS Analytical Method B: rt=2.27 minutes, m/z 242.10 [M+H]⁺.

S1-T3: 5-{[(2S)-2-phenylpyrrolidin-1-yl]carbonyl}-1H-1,2,4-triazole (Off White solid, 25.41 mg, 20.56%)

¹H NMR (400 MHz, DMSO-d₆ at 373K) δ 14.15 (bs, 1H), 8.07-8.28 (m, 1H), 7.20-7.25 (m, 5H), 5.28 (bs, 1H), 3.82-4.07 (m, 2H), 2.33 (bs, 1H), 1.86 (bs, 3H).

LCMS Analytical Method B: rt=2.31 minutes, m/z 243.10 [M+H]⁺.

S1-T4: 5-{[(2S)-2-phenylpyrrolidin-1-yl]carbonyl}-1,2-oxazole (Off White solid, 61.69 mg, 37.43%)

¹H NMR (400 MHz, DMSO-d₆ at 373K) δ 8.43-8.66 (bd, 1H), 7.21-7.30 (m, 5H), 6.50-6.92 (bd, 1H), 5.27 (bs, 1H), 3.82-4.07 (m, 2H), 2.33 (bs, 1H), 1.86 (bs, 3H).

LCMS Analytical Method B: rt=2.33 minutes, m/z 243.10 [M+H]⁺.

S1-T5: 3-{[(2S)-2-phenylpyrrolidin-1-yl]carbonyl}-1,2-oxazole (Off White solid, 15.07 mg, 12.19%)

¹H NMR (400 MHz, DMSO-d₆ at 373K) δ 8.95 (bs, 1H), 7.07-7.29 (m, 5H), 6.77 (bs, 1H), 5.25 (bs, 1H), 3.82-3.93 (m, 2H), 2.40 (bs, 1H), 1.86 (bs, 3H).

LCMS Analytical Method B: rt=2.40 minutes, m/z 243.10 [M+H]⁺.

S1-T10: 4-{[(2S)-2-phenylpyrrolidin-1-yl]carbonyl}-1,3-oxazole (Off White solid, 26.43 mg, 21.38%)

¹H NMR (400 MHz, DMSO-d₆ at 373K) δ 8.29 (bs, 2H), 7.18-7.30 (m, 5H), 5.44 (bs, 1H), 3.92 (bs, 2H), 2.33 (bs, 1H), 1.86 (bs, 3H).

LCMS Analytical Method B: rt=1.67 minutes, m/z 243.10 [M+H]⁺.

S1-T11: 2-{[(2S)-2-phenylpyrrolidin-1-yl]carbonyl}pyridine (Brown gum, 32.77 mg, 25.46%)

¹H NMR (400 MHz, DMSO-d₆ at 373K) δ 8.40-8.62 (m, 1H), 6.97-7.88 (m, 8H), 5.47 (bs, 1H), 3.75-3.90 (m, 2H), 2.33 (bs, 1H), 1.86 (bs, 3H).

LCMS Analytical Method B: rt=2.56 minutes, m/z 253.15 [M+H]⁺.

S1-T12: 3-{[(2S)-2-phenylpyrrolidin-1-yl]carbonyl}pyridine (Off White solid, 40.38 mg, 31.37%)

¹H NMR (400 MHz, DMSO-d₆ at 373K) δ 8.56 (bs, 2H), 7.78 (bs, 1H), 7.19-7.28 (m, 6H), 5.11 (bs, 1H), 3.62-3.80 (m, 2H), 2.37 (bs, 1H), 1.87 (bs, 3H).

LCMS Analytical Method B: rt=2.20 minutes, m/z 253.15 [M+H]⁺.

S1-T13: 4-{[(2S)-2-phenylpyrrolidin-1-yl]carbonyl}pyridine (White Gum, 13.36 mg, 10.38%)

¹H NMR (400 MHz, DMSO-d₆ at 373K) δ 8.63 (bs, 2H), 7.20-7.28 (m, 7H), 5.11 (bs, 1H), 3.75 (bs, 2H), 2.32 (bs, 1H), 1.87 (bs, 3H).

LCMS Analytical Method B: rt=2.03 minutes, m/z 253.15 [M+H]⁺.

S1-T14: 4-chloro-5-{[(2S)-2-phenylpyrrolidin-1-yl]carbonyl}-1H-pyrazole (Off white solid, 45.19 mg, 32.12%)

¹H NMR (400 MHz, DMSO-d₆ at 373K) δ 13.10 (bs, 1H), 7.79-7.97 (m, 1H), 7.18-7.26 (m, 5H), 5.28 (bs, 1H), 3.80 (bs, 2H), 2.32 (bs, 1H), 1.87 (bs, 3H).

LCMS Analytical Method B: rt=2.59 minutes, m/z 276.14 [M+H]⁺.

S1-T15: 4-chloro-5-{[(2S)-2-(2-fluorophenyl)pyrrolidin-1-yl]carbonyl}-1H-pyrazole (white solid, 25.03 mg, 21.9%)

¹H NMR (400 MHz, DMSO-d₆ at 373K) δ 13.15 (bs, 1H), 7.89 (bs, 1H), 7.11-7.24 (m, 4H), 5.40 (bs, 1H), 3.80 (bs, 2H), 2.36 (bs, 1H), 1.92 (bs, 3H).

LCMS Analytical Method B: rt=2.31 minutes, m/z 294.15 [M+H]⁺.

S1-T16: 4-chloro-5-{[(2S)-2-(2-methoxyphenyl)pyrrolidin-1-yl]carbonyl}-1H-pyrazole (off white solid, 30.48 mg, 23.59%)

¹H NMR (400 MHz, DMSO-d₆ at 373K) δ 13.11 (bs, 1H), 7.89 (bs, 1H), 6.86-7.17 (m, 4H), 5.43 (bs, 1H), 3.69-3.83 (m, 6H), 2.30 (bs, 1H), 1.85 (bs, 3H).

LCMS Analytical Method B: rt=2.64 minutes, m/z 306.15 [M+H]⁺.

S1-T17: 2-[(2S)-1-[(4-chloro-1H-pyrazol-5-yl)carbonyl]pyrrolidin-2-yl]pyridine (off white solid, 49.22 mg, 24.92%)

¹H NMR (400 MHz, DMSO-d₆ at 373K) δ 11.56 (bs, 1H), 8.47 (bs, 1H), 7.67-7.88 (m, 2H), 7.18-7.28 (m, 2H), 5.28 (bs, 1H), 3.71-3.83 (m, 2H), 2.36 (bs, 1H), 1.93 (bs, 3H).

LCMS Analytical Method B: rt=1.96 minutes, m/z 277.15 [M+H]⁺.

S1-T22: 5-{[(2S)-2-phenylpyrrolidin-1-yl]carbonyl}-1H-imidazole (off white solid, 56.09 mg, 45.56%)

¹H NMR (400 MHz, DMSO-d₆ at 373K) δ 12.10 (bs, 1H), 7.59 (s, 1H), 8.47 (bs, 1H), 7.18-7.39 (m, 6H), 5.49 (bs, 1H), 2.96 (bs, 2H), 2.33 (bs, 1H), 1.85 (bs, 3H).

LCMS Analytical Method B: rt=2.04 minutes, m/z 242.10 [M+H]⁺.

S1-T23: 5-{[(2S)-2-[2-(propan-2-yl)phenyl]pyrrolidin-1-yl]carbonyl}-1H-imidazole (Brown Solid, 34.36 mg, 30.56%)

¹H NMR (400 MHz, DMSO-d₆ at 373K) δ 12.12 (bs, 1H), 7.00-7.59 (m, 6H), 5.57 (bs, 1H), 4.01 (bs, 2H), 3.29-3.36 (m, 1H), 2.33 (bs, 1H), 1.85 (bs, 3H), 1.25-1.29 (m, 6H).

LCMS Analytical Method B: rt=2.37 minutes, m/z 284.22 [M+H]⁺.

S1-T24: 5-{[(2S)-2-(2-fluorophenyl)pyrrolidin-1-yl]carbonyl}-1H-imidazole (off white solid, 23.69 mg, 20.47%)

¹H NMR (400 MHz, DMSO-d₆ at 373K) δ 12.25 (bs, 1H), 7.61 (s, 1H), 7.06-7.45 (m, 5H), 5.56 (bs, 1H), 4.02 (bs, 2H), 2.33 (bs, 1H), 1.85 (bs, 3H).

LCMS Analytical Method B: rt=1.95 minutes, m/z 260.17 [M+H]⁺.

S1 Series Ester Hydrolysis Followed by Amidation with (S)-2 Phenylpyrrolidine

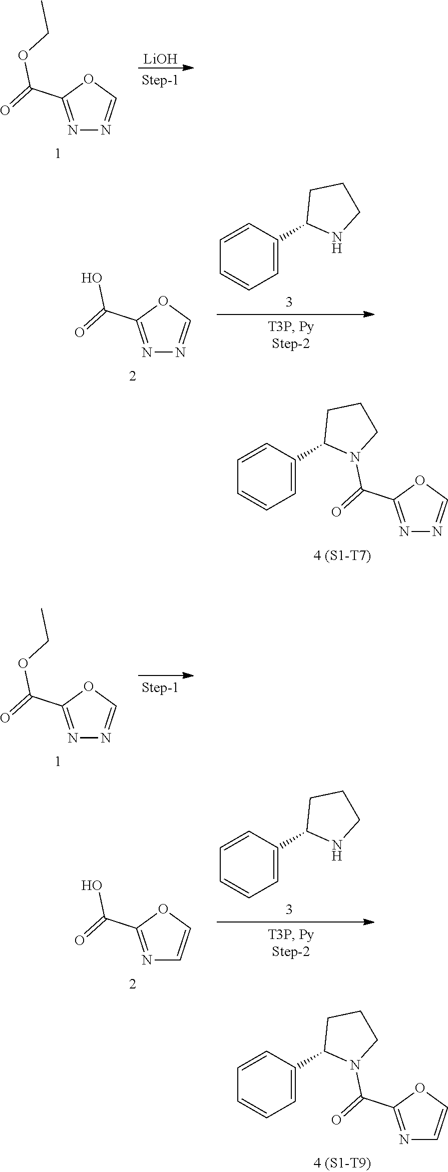

### General Procedure for Ester Hydrolysis

To a stirred solution of ester (250 mg, 1.0 eq) in tetrahydrofuran (THF):water (1.5 ml, 1:1) at ice cold condition, a solution of lithium hydroxide (0.51 mmol, 1.0 eq) in water was added. Reaction mixture was stirred at ice cold condition for 4 h. TLC was checked, 1 M of HCl was added until the pH reached pH=5 & the resultant mixture was lyophilised to afford desired acid contaminated with LiCl salt. The crude product was used for the amidation step.

### General Procedure for Amidation Using T3P Reagent

To a stirred solution of acid (0.51 mmol, 1 eq) and(S)-2 Phenylpyrrolidine (0.51 mmol, 1.0 eq) in DMF (0.5 ml) were added T3P (50% in ethyl acetate, 1.531 mmol, 3 eq) and pyridine (3.061 mmol, 6.0 eq) and heated at 70° C. for 3 h.

The reaction mixture was diluted with ethyl acetate (20 ml) and washed with water. The organic layer was washed with brine, dried over anhydrous Na₂SO₄, filtered and concentrated. The crude material was purified by prep-TLC (in 40% EA+HEX) followed by lyophilization to afford desired compound.

S1-T7: 2-[(2S)-1-[(4-chloro-1H-pyrazol-5-yl)carbonyl]pyrrolidin-2-yl]pyridine (off white solid, 49.22 mg, 24.92%)

¹H NMR (400 MHz, DMSO-d₆ at 373K) δ 9.03-9.28 (bd, 1H), 7.10-7.30 (m, 5H), 5.27-5.66 (bs, 1H), 4.02 (bs, 2H), 2.33 (bs, 1H), 1.89 (bs, 3H).

LCMS Analytical Method B: rt=2.21 minutes, m/z 244.12 [M+H]⁺.

S1-T9: 2-{[(2S)-2-phenylpyrrolidin-1-yl]carbonyl}-1,3-oxazole (off white solid, 33.23 mg, 26.88%)

¹H NMR (400 MHz, DMSO-d₆ at 373K) δ 7.96-8.20 (bd, 1H), 7.10-7.42 (m, 6H), 5.25-5.85 (bd, 1H), 4.12 (bd, 2H), 2.38 (bs, 1H), 1.95 (bs, 3H).

LCMS Analytical Method B: rt=2.57 minutes, m/z 243.2 [M+H]⁺.

### Synthesis of N-ethyl-2-fluoro-N-[1-(1H-imidazol-4-ylmethyl)-2,3-dihydro-1H-indol-5-yl]benzamide (S2-T1)

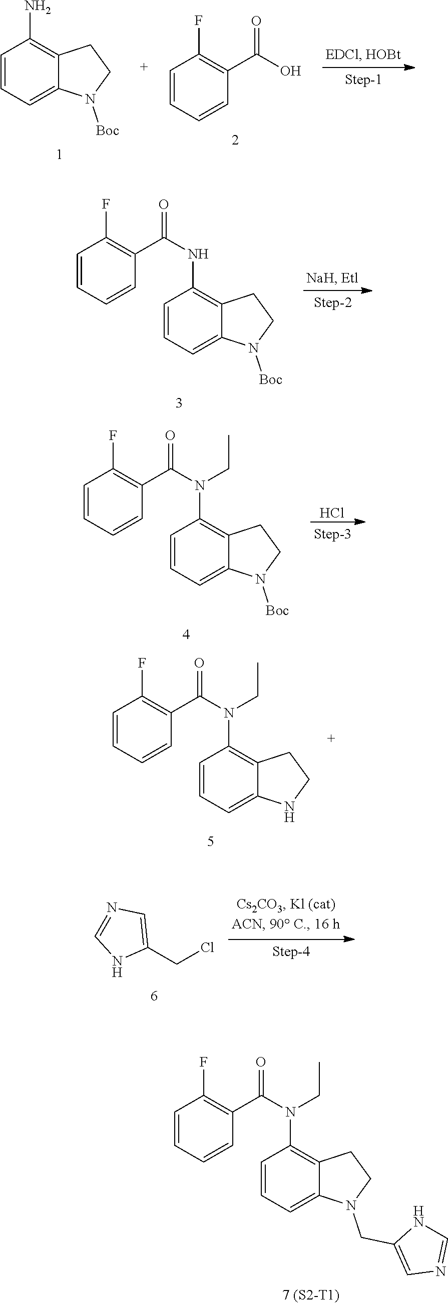

Step-1: Synthesis of tert-butyl 4-[(2-fluorobenzene)amido]-2,3-dihydro-1H-indole-1-carboxylate (CR652-18239-87-1-P)

To a stirred solution of 2-fluorobenzoic acid (299.145 mg, 2.137 mmol) in dichloromethane (DCM) (3 ml) at 0° C. were added triethylamine (1.236 ml, 8.547 mmol), EDC·HCl (614.423 mg, 3.205 mmol) and HOBT (433.077 mg, 3.205 mmol) and stirred for 15 minutes. Then a solution of tert-butyl 4-aminoindoline-1-carboxylate (500.0 mg, 2.137 mmol) in DCM (2 ml) was added dropwise to the reaction mixture. The reaction mixture was stirred at 23° C. for 16 h. The reaction mixture was diluted with DCM (50 mL) and washed water, saturated NaHCO₃ and brine solution. The organic layer was washed with brine, dried over anhydrous Na₂SO₄, filtered and concentrated. The crude material was purified by combi-flash chromatography to get desired tert-butyl 4-[(2-fluorobenzene)amido]-2,3-dihydro-1H-indole-1-carboxylate (650 mg, 85.36% yield).

Step-2: Synthesis of tert-butyl 4-[N-ethyl(2-fluorobenzene)amido]-2,3-dihydro-1H-indole-1-carboxylate (CR652-18239-93-1-P)

To a stirred solution of tert-butyl 4-[(2-fluorobenzene)amido]-2,3-dihydro-1H-indole-1-carboxylate (650 mg, 1.825 mmol) in THF (10 ml), NaH (60% suspension in mineral oil, 180 mg, 3.651 mmol) was added and the mixture was stirred at 0° C. for 45 minutes. Ethyl Iodide (0.293 ml, 3.651 mmol) was added and the reaction mixture was stirred at 0° C. for 30 minutes followed by at 23° C. for 16 h. The reaction mixture was quenched with saturated NH₄Cl solution and extracted with ethyl-acetate (50 ml). The combined organic layer was washed with brine, dried over anhydrous Na₂SO₄ filtered and concentrated in vacuo to afford tert-butyl 4-[N-ethyl(2-fluorobenzene)amido]-2,3-dihydro-1H-indole-1-carboxylate (610 mg, 81.49% yield) as brown gum.

Step-3: Synthesis of N-ethyl-2-fluoro-N-(indolin-4-yl)benzamide (CR652-18239-95-1-C1)

To a stirred solution of tert-butyl 4-[N-ethyl(2-fluorobenzene)amido]-2,3-dihydro-1H-indole-1-carboxylate (2 g, 5.208 mmol) in dioxane (10 ml) at ice cold condition, HCl in dioxane (4M, 15 ml) was added dropwise and the reaction mixture was stirred at 23° C. for 16 h. The reaction mass was neutralized with saturated NaHCO₃ and extracted with ethyl acetate, dried over anhydrous Na₂SO₄, filtered and concentrated in vacuo to afford N-ethyl-2-fluoro-N-(indolin-4-yl)benzamide (1.2 g, 81.03%) as an orange solid.

Step-4: Synthesis of N-ethyl-2-fluoro-N-[1-(1H-imidazol-4-ylmethyl)-2,3-dihydro-1H-indol-5-yl]benzamide (CR652-18239-98-1-P)

To a stirred solution of N-ethyl-2-fluoro-N-(indolin-4-yl)benzamide (200 mg, 0.704 mmol, 1.0 eq) in dry acetonitrile (7 ml), Cs₂CO₃ (457.746 mg, 1.408 mmol, 2.0 eq) was added followed by 5-(chloromethyl)-1H-imidazole (215 mg, 1.408 mmol, 2.0 eq) in acetonitrile with cat KI. The reaction mass was heated at 80° C. for 3 hours. The reaction mass was filtered and the acetonitrile was evaporated. The afforded residue was dissolved in ethyl acetate and washed with brine and the crude product was purified by Prep-HPLC to get desired N-ethyl-2-fluoro-N-[1-(1H-imidazol-4-ylmethyl)-2,3-dihydro-1H-indol-5-yl]benzamide (47.84 mg, 18.64% yield) as a off white solid.

S2-T1: N-ethyl-2-fluoro-N-[1-(1H-imidazol-4-ylmethyl)-2,3-dihydro-1H-indol-5-yl]benzamide (off white solid, 47.84 mg, 18.64%)

¹H NMR (400 MHz, DMSO-d₆ at 373K) δ 11.63 (bs, 1H), 7.49 (s, 1H), 6.80-7.36 (m, 6H), 6.34-6.42 (bd, 2H), 4.10 (bs, 2H), 3.72 (bs, 1H), 3.31 (bs, 2H), 2.78 (s, 2H), 1.12 (s, 3H).

LCMS Analytical Method B: rt=2.21 minutes, m/z 365.2 [M+H]⁺.

### Synthesis of N-ethyl-2-fluoro-N-[1-(1H-imidazol-5-ylmethyl)-2-oxo-1,2-dihydroquinoxalin-5-yl]benzamide (S2-T2)

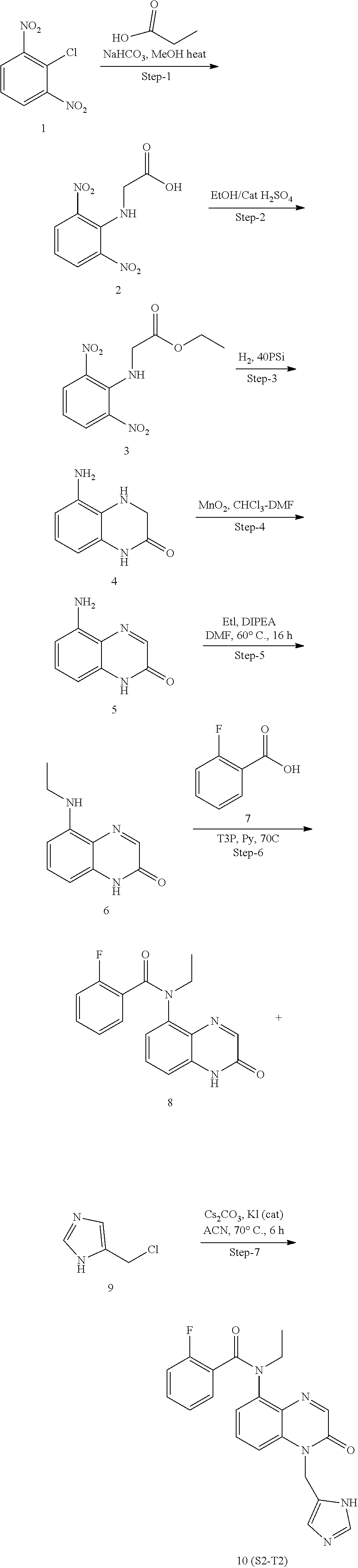

Step-1: Synthesis of 2-[(2,6-dinitrophenyl)amino]acetic acid (CR652-18239-40-1-C)

Glycine (2.22 g, 29.622 mmol, 1.2 eq) and NaHCO₃ (5.12 g, 54.307 mmol, 2.2 eq) were dissolved in water (20 ml) and a methanol solution (50 ml) of 2-chloro-1,3-dinitrobenzene (5.0 g, 24.685 mmol, 1.0 eq) was added dropwise and heated at 80° C. for 6 h. The reaction mixture was concentrated under reduce pressure & pH adjusted to 3-4 with 1M HCl. The aqueous part was extracted with 5% MeOH-ethyl acetate and washed with brine solution and concentrated to get the desired 2-[(2,6-dinitrophenyl)amino]acetic acid (4.25 g, 80.12% yield) as a yellow solid.

Step-2: Synthesis of ethyl 2-[(2,6-dinitrophenyl)amino]acetate (CR652-18239-43-1-P)

To a stirred solution of 2-[(2,6-dinitrophenyl)amino]acetic acid (4.0 g, 4.147 mmol) in ethanol (60 ml) was added concentrated H₂SO₄ (2.5 ml) and the mixture was heated under reflux for 16 h. The reaction mixture was concentrated under reduced pressure, saturated NaHCO₃ solution was added to the residue and extracted with 10% MeOH-DCM to afford ethyl 2-[(2,6-dinitrophenyl)amino]acetate (3.8 g, 78.78% yield) as a yellow solid.

Step-3: Synthesis of 5-amino-1,2,3,4-tetrahydroquinoxalin-2-one (CR652-18239-47-1-C1)

To a stirred solution of ethyl 2-[(2,6-dinitrophenyl)amino]acetate (3.5 g, 13.011 mmol) in ethanol (30 ml) & dimethylformamide (10 ml) was added 10% Pd—C (˜50% moist, 1.7 g) under nitrogen and hydrogenated under balloon pressure at 23° C. for 16 hours. The reaction mixture was filtered through celite and the celite pad was washed with hot 10% DMF-ethanol (7-8 times) to afford 5-amino-1,2,3,4-tetrahydroquinoxalin-2-one (1.7 g, 82.43% yield) as a brownish gum. The crude was carried forward without further purification.

Step-4: Synthesis of 5-amino-1,2-dihydroquinoxalin-2-one (CR652-18239-107-1-P)

To a stirred solution of 5-amino-1,2,3,4-tetrahydroquinoxalin-2-one (2.5 g, 15.337 mmol) in CHCl₃ (50 ml) and dimethylformamide (10 ml), MnO₂ (6.0 g, 69.018 mmol, 4.5 eq) was added and stirred at 23° C. for 16 hours. The reaction mixture was filtered through celite and washed with hot 30% DMF-ethanol (7-8 times) and evaporated to afford 5-amino-1,2-dihydroquinoxalin-2-one (1.9 g, 76.87% yield) as deep yellow solid.

Step-5: Synthesis of 5-(ethylamino)-1,2-dihydroquinoxalin-2-one (CR652-18239-108-1-P4)

To a stirred solution of 5-amino-1,2-dihydroquinoxalin-2-one (1.9 g, 11.801 mmol) in dry THF (25 ml) was added DIPEA (4.383 ml, 23.602 mmol, 2.0 eq) and stirred for 20 minutes. Then ethyl iodide (1.044 ml, 12.981 mmol, 1.1 eq) in THF (5 ml) was added dropwise and the reaction mixture was stirred at 70° C. for 16 h. The reaction was monitored by LCMS, and showed 50% conversion of SM. Another 1.1 eq of ethyl iodide was added and heated for 16 h. The reaction mixture was quenched with saturated NH₄Cl and extracted with MTBE (2×100 ml). The combined organic layer was washed with brine, dried over anhydrous Na₂SO₄, filtered and concentrated in vacuo to afford an orange gum. The crude material was purified by combi-flash chromatography to afford 5-(ethylamino)-1,2-dihydroquinoxalin-2-one (1.2 g, 62.18% yield) as a light yellow solid.

Step-6: Synthesis of N-ethyl-2-fluoro-N-(2-oxo-1,2-dihydroquinoxalin-5-yl)benzamide (CR652-18239-154-1-P)

To a stirred solution of 2-fluorobenzoic acid (773 mg, 5.526 mmol, 1.5 eq) and 5-(ethylamino)-1,2-dihydroquinoxalin-2-one (700 mg, 3.684 mmol, 1.0 eq) in DMF (1 ml) were added T3P (˜50% in ethyl acetate, 1.531 mmol, 3 eq) and pyridine (3.061 mmol, 6.0 eq). The resulting mixture was heated at 70° C. for 3 hours. The reaction mixture was diluted with ethyl-acetate (20 ml) and washed with water. The organic layer was washed with saturated NaHCO₃ solution, brine, dried over anhydrous Na₂SO₄, filtered and concentrated in vacuo to afford N-ethyl-2-fluoro-N-(2-oxo-1,2-dihydroquinoxalin-5-yl)benzamide (300 mg, 64% crude yield) as brown gum.

Step-7: Synthesis of N-ethyl-2-fluoro-N-[1-(1H-imidazol-5-ylmethyl)-2-oxo-1,2 dihydroquinoxalin-5-yl]benzamide (CR652-18239-157-1-P)

To a stirred solution of N-ethyl-2-fluoro-N-(indolin-4-yl)benzamide (300.0 mg, 0.965 mmol, 1.0 eq) in dry acetonitrile (7 ml) was added Cs₂CO₃ (627.01 mg, 1.929 mmol, 2.0 eq) followed by 5-(chloromethyl)-1H-imidazole (177.106 mg, 1.158 mmol, 1.2 eq) in acetonitrile with cat KI. The reaction mass was heated at 80° C. for 3 h. The reaction mass was filtered and acetonitrile was evaporated. The crude material was purified by prep-HPLC (using NH₄HCO₃ buffer) followed by lyophilisation to afford N-ethyl-2-fluoro-N-[1-(1H-imidazol-5-ylmethyl)-2-oxo-1,2-dihydroquinoxalin-5-yl]benzamide (26.0 mg, 6.89% yield) as a light yellow solid.

S2-T2: N-ethyl-2-fluoro-N-[1-(1H-imidazol-5-ylmethyl)-2-oxo-1,2 dihydroquinoxalin-5-yl]benzamide (light yellow solid, 26.0 mg, 6.89%)

¹H NMR (400 MHz, DMSO-d₆ at 373K) δ 11.68 (bs, 1H), 8.28 (s, 1H), 7.73 (bs, 1H), 7.47-7.69 (m, 2H), 7.17-7.25 (m, 3H), 6.91-7.00 (m, 2H), 6.82 (s, 1H), 5.26 (s, 2H), 3.89 (bs, 2H), 1.12 (t, 3H, J=8 Hz).

LCMS Analytical Method B: rt=2.05 minutes, m/z 392.2 [M+H]⁺.

### Synthesis of S2-T3, S2-T4, S2-T5, S2-T6 & S2-T7

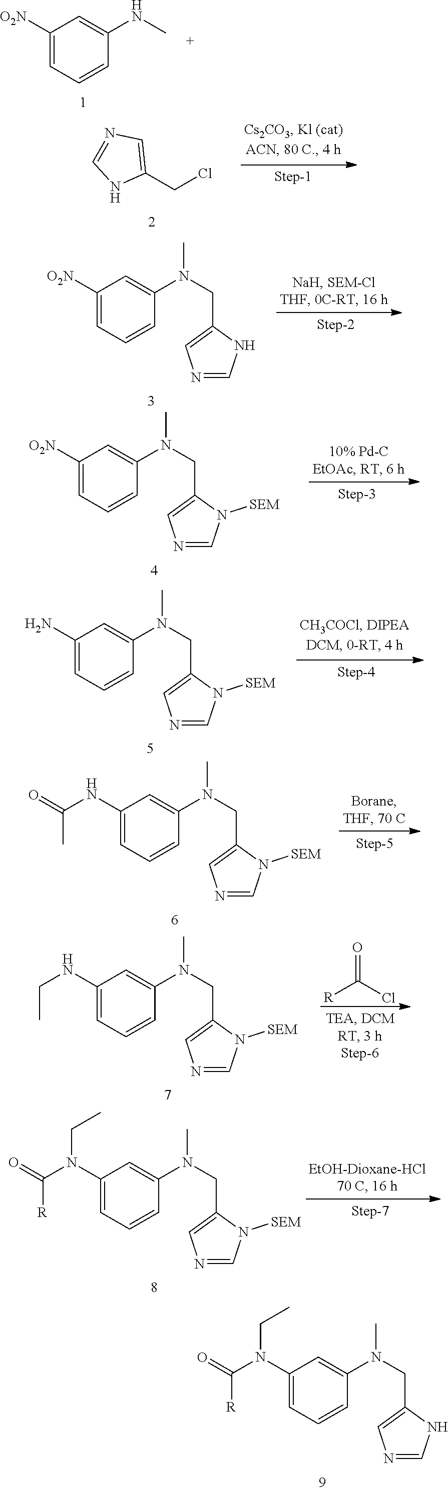

Step-1: Synthesis of N-((1H-imidazol-4-yl)methyl)-N-methyl-3-nitroaniline (CR652-18239-120-1-P)

To a stirred solution of N-methyl-3-nitro-aniline (10 g, 26.29 mmol, 1 eq) in dry acetontrile (20 ml) was added caesium carbonate (3.28 g, 34.18 mmol, 1.3 eq) followed by 4-(chloromethyl)-1H-imidazole·hydrochloride (3.28 g, 34.18 mmol, 1.3 eq) in acetonitrile with cat KI. The reaction mass was heated at 80° C. for 3 h. The reaction mixture was filtered and concentrated. Ethyl acetate was added (75 ml) and washed with water (100 ml). The aqueous part was further extracted with ethyl acetate (2×100 ml). The combined organic part was washed with brine, dried over anhydrous sodium sulphate, filtered and solvent was evaporated in vacuo to afford crude product as an orange oil. The crude was triturated to afforded N-((1H-imidazol-4-yl)methyl)-N-methyl-3-nitroaniline as an orange gum (12.1 g, 85.08%).

Step-2: Synthesis of N-methyl-3-nitro-N-((1-((2-(trimethylsilyl)ethoxy)methyl)-1H-imidazol-4-yl)methyl)aniline and N-methyl-3-nitro-N-((1-((2-(trimethylsilyl)ethoxy)methyl)-1H-imidazol-5-yl)methyl)aniline 1:1 Regioisomeric Mixture (CR652-18239-148-1-P2)

To a stirred suspension of NaH (60% suspension in oil, 1.26 g, 31.39 mmol) in THF (20 ml) at 0° C. was slowly added a solution of N-(1H-imidazol-5-ylmethyl)-N-methyl-3-nitro-aniline (6.08 g, 26.16 mmol) in THF (60 ml) and the mixture was stirred at 0° C. for 45 minutes. A THF solution of 2-(Trimethylsilyl)ethoxymethyl chloride (6.95 ml, 39.24 mmol) was then added and the reaction mixture was stirred at 0° C. for 30 minutes and at 23° C. for 2 hours. The reaction mixture was quenched with water and concentrated to remove THF. The resulting suspension was partitioned between ethyl acetate (150 ml) and NaOH (1 M, 100 ml). The aqueous layer was further extracted with ethyl acetate (50 ml) then the organics were combined, washed with brine, dried over sodium sulphate, filtered and concentrated in vacuo to afford crude product as an orange solid. Purification by flash column chromatography (silica column, DCM 0% to 10% gradient of a solution) afforded a 1:1 regioisomeric mixture of the title compounds as an orange oil (3.74 g, 40%).

LCMS Analytical Method A: rt=1.52, 1.58 minutes, m/z 363.2 [M+H]⁺.

Step-3: Synthesis of N¹-methyl-N¹-((1-((2-(trimethylsilyl)ethoxy)methyl)-1H-imidazol-4-yl)methyl)benzene-1,3-diamine and N¹-methyl-N¹-((1-((2-(trimethylsilyl)ethoxy)methyl)-1H-imidazol-5-yl)methyl)benzene-1,3-diamine 1:1 Regioisomeric Mixture (CR652-18239-149-1-C1)

A solution of N-methyl-3-nitro-N-((1-((2-(trimethylsilyl)ethoxy)methyl)-1H-imidazol-4-yl)methyl)aniline and N-methyl-3-nitro-N-((1-((2-(trimethylsilyl)ethoxy)methyl)-1H-imidazol-5-yl)methyl)aniline (1:1 regioisomeric mixture, 3.6 g) in ethyl acetate (100 ml) was hydrogenated using 10% Pd/C (˜50% moist, 1.8 g) under balloon pressure. The reaction mass was filtered using celite bed followed by evaporation of solvent in vacuo to afford crude product as regioisomeric mixture as a yellow oil (2.6 g, 80%).

LCMS Analytical Method A: rt=1.29 minutes, m/z 333.2 [M+H]⁺.

Step-4: Synthesis of N-(3-(methyl((1-((2-(trimethylsilyl)ethoxy)methyl)-1H-imidazol-4-yl)methyl)amino)phenyl) acetamide and N-(3-(methyl((1-((2-(trimethylsilyl)ethoxy)methyl)-1H-imidazol-5-yl)methyl)amino)phenyl) acetamide 1:1 Regioisomeric Mixture (CR652-18239-150-1-C3)

N¹-methyl-N¹-((1-((2-(trimethylsilyl)ethoxy)methyl)-1H-imidazol-4-yl)methyl)benzene-1,3-diamine and N¹-methyl-N¹-((1-((2-(trimethylsilyl)ethoxy)methyl)-1H-imidazol-5-yl)methyl)benzene-1,3-diamine (1:1 regioisomeric mixture, 2.5 g, 7.534 mmol) was dissolved in DCM (20 ml) and DIPEA (5.4 ml, 30.120 mmol) was added. The solution was cooled to 0° C., acetyl chloride (0.7 ml, 9.789 mmol) was added and the reaction was stirred at 0° C. for 1 h. The reaction mixture was diluted with DCM (20 ml) and washed with water (20 ml), saturated sodium bicarbonate solution (20 ml) and brine. Organic part was dried over anhydrous sodium sulphate, filtered and solvent was evaporated in vacuo to afford as a pale brown oil (2.39 g, 83%).

LCMS Analytical Method A: rt=1.49 minutes, m/z 375.2 [M+H]⁺.

Step-5: Synthesis of N¹-ethyl-N³-methyl-N³-((1-((2-(trimethylsilyl)ethoxy)methyl)-1H-imidazol-4-yl)methyl)benzene-1,3-diamine and N¹-ethyl-N³-methyl-N³-((1-((2-(trimethylsilyl)ethoxy)methyl)-1H-imidazol-5-yl)methyl)benzene-1,3-diamine 1:1 Regioisomeric Mixture (CR652-18239-153-1-P)

N-(3-(methyl((1-((2-(trimethylsilyl)ethoxy)methyl)-1H-imidazol-4-yl)methyl)amino)phenyl) acetamide and N-(3-(methyl((1-((2-(trimethylsilyl)ethoxy)methyl)-1H-imidazol-5-yl)methyl)amino)phenyl) acetamide (1:1 regioisomeric mixture, 2.2 g, 5.888 mmol) was dissolved in THF (7.5 ml) and borane-THF complex solution (1 M, 35 ml, 35.294 mmol) was added slowly at room temperature. The reaction was heated at 70° C. for 16 hours. MeOH (50 ml) was added and heating was continued at 70° C. for another 24 hours. Solvent was evaporated in vacuo and the resulting brown oil loaded onto silica gel and purified by flash column chromatography (silica column, DCM 0% to 10% gradient of a solution) to afford the title compound regioisomeric mixture as a pale brown gum (1.8 g, 85%).

LCMS Analytical Method A: rt=1.54 minutes, m/z 361.2 [M+H]⁺.

### General Procedure for Amidation Reaction Using Acid Chloride & Subsequent SEM Deprotection

N¹-ethyl-N³-methyl-N³-((1-((2-(trimethylsilyl)ethoxy)methyl)-1H-imidazol-4-yl)methyl)benzene-1,3-diamine and N¹-ethyl-N³-methyl-N³-((1-((2-(trimethylsilyl) ethoxy)methyl)-1H-imidazol-5-yl)methyl)benzene-1,3-diamine (1:1 regioisomeric mixture, 300 mg, 0.833 mmol) was dissolved in DCM (2.5 ml) and DIPEA (0.21 ml, 1.21 mmol) was added. The reaction was stirred at 0° C. for 30 minutes then aryl acid chloride (1.24 mmol) was added and the reaction mixture was allowed to warm to 23° C. over 16 h. The reaction mixture was diluted with DCM (20 ml) and washed with saturated NaHCO₃ solution. The aqueous part was re-extracted with DCM (2×15 ml) and the combined organic part was dried over anhydrous sodium sulphate, filtered and concentrated in vacuo to afford SEM-protected amide.

SEM deprotection was done using EtOH/4 M dioxane-HCl at 23° C. for 16 hours. Purification by preparative HPLC (basic NH₄HCO₃ buffer) followed by lyophilization afforded the desired compound.

S2-T3: N-ethyl-N-[3-[1H-imidazol-5-ylmethyl(methyl)amino]phenyl]pyridine-2-carboxamide (Brown sticky solid, 19.91 mg, 18.44%)

¹H NMR (400 MHz, DMSO-d₆ at 373K) δ 11.57 (bs, 1H), 8.34 (s, 1H), 7.64 (d, 1H, J=8 Hz), 7.48 (s, 1H), 7.32 (d, 1H, J=8 Hz), 7.21 (s, 1H), 6.98 (bs, 1H), 6.70 (bs, 1H), 6.57-6.59 (d, 1H, J=8 Hz), 6.51 (s, 1H), 6.36 (bs, 1H), 4.27 (s, 2H), 3.82 (t, 3H, J=8 Hz), 2.82 (s, 3H), 1.14 (t, 3H, J=8 Hz).

LCMS Analytical Method B: rt=2.01 minutes, m/z 336.22 [M+H]⁺.

S2-T4: N-ethyl-4-fluoro-N-[3-[1H-imidazol-5-ylmethyl(methyl)amino]phenyl]benzamide (Light brown sticky gum, 15.01 mg, 11.76%)

¹H NMR (400 MHz, DMSO-d₆ at 373K) δ 11.60 (bs, 1H), 7.49 (s, 1H), 7.26-7.29 (m, 2H), 7.21 (s, 1H), 6.92-7.00 (m, 3H), 6.69 (s, 1H), 6.49-6.60 (m, 1H), 6.30 (s, 1H), 4.28 (s, 2H), 3.77-3.82 (m, 2H), 2.83 (s, 3H), 1.10 (t, 3H, J=8 Hz).

LCMS Analytical Method B: rt=2.28 minutes, m/z 353.2 [M+H]⁺.

S2-T5: N-ethyl-3-fluoro-N-[3-[1H-imidazol-5-ylmethyl(methyl)amino]phenyl]benzamide (Brown sticky solid, 6.51 mg, 5.25%)

¹H NMR (400 MHz, DMSO-d₆ at 298K) δ 11.82 (bs, 1H), 7.52 (s, 1H), 7.21 (bs, 1H), 6.97-7.07 (m, 4H), 6.69 (bs, 1H), 6.57 (bs, 2H), 6.33 (bs, 1H), 4.30 (s, 2H), 3.79-3.81 (m, 2H), 2.84 (s, 3H), 1.08 (t, 3H, J=8 Hz).

LCMS Analytical Method B: rt=2.29 minutes, m/z 353.2 [M+H]⁺.

S2-T6: N-ethyl-N-[3-[1H-imidazol-5-ylmethyl(methyl)amino]phenyl]tetrahydropyran-4-carboxamide (Brown sticky solid, 22.52 mg, 20.74%)

¹H NMR (400 MHz, DMSO-d₆ at 373K) δ 11.63 (bs, 1H), 7.48 (s, 1H), 7.19-7.21 (m, 1H) 6.80 (bs, 2H), 6.61 (s, 1H), 6.46 (s, 1H), 4.41 (s, 2H), 3.74-3.76 (m, 2H), 3.57-3.60 (m, 2H), 1.58-1.64 (m, 2H), 1.37-1.41 (m, 2H), 1.01 (t, 3H, J=8 Hz).

LCMS Analytical Method B: rt=2.06 minutes, m/z 343.2 [M+H]⁺.

S2-T7: N-ethyl-N-[3-[1H-imidazol-5-ylmethyl(methyl)amino]phenyl]-1-methyl-piperidine-2-carboxamide (Brown sticky gum, 3.61 mg, 3.39%)

¹H NMR (400 MHz, DMSO-d₆ at 373K) δ 11.59 (bs, 1H), 7.48 (s, 1H), 7.18-7.22 (m, 1H) 6.79 (bs, 2H), 6.56 (s, 1H), 6.41 (s, 1H), 4.40 (s, 2H), 3.55-3.72 (m, 2H), 2.80-2.83 (m, 3H), 2.66 (s, 1H), 2.04 (s, 3H), 1.82 (s, 1H), 1.27-1.59 (m, 6H), 0.99 (t, 3H, J=8 Hz).

LCMS Analytical Method B: rt=1.77 minutes, m/z 356.2 [M+H]⁺.

### Synthesis of S2-T8, S2-T9, S2-T10, S2-T11 & S2-T12

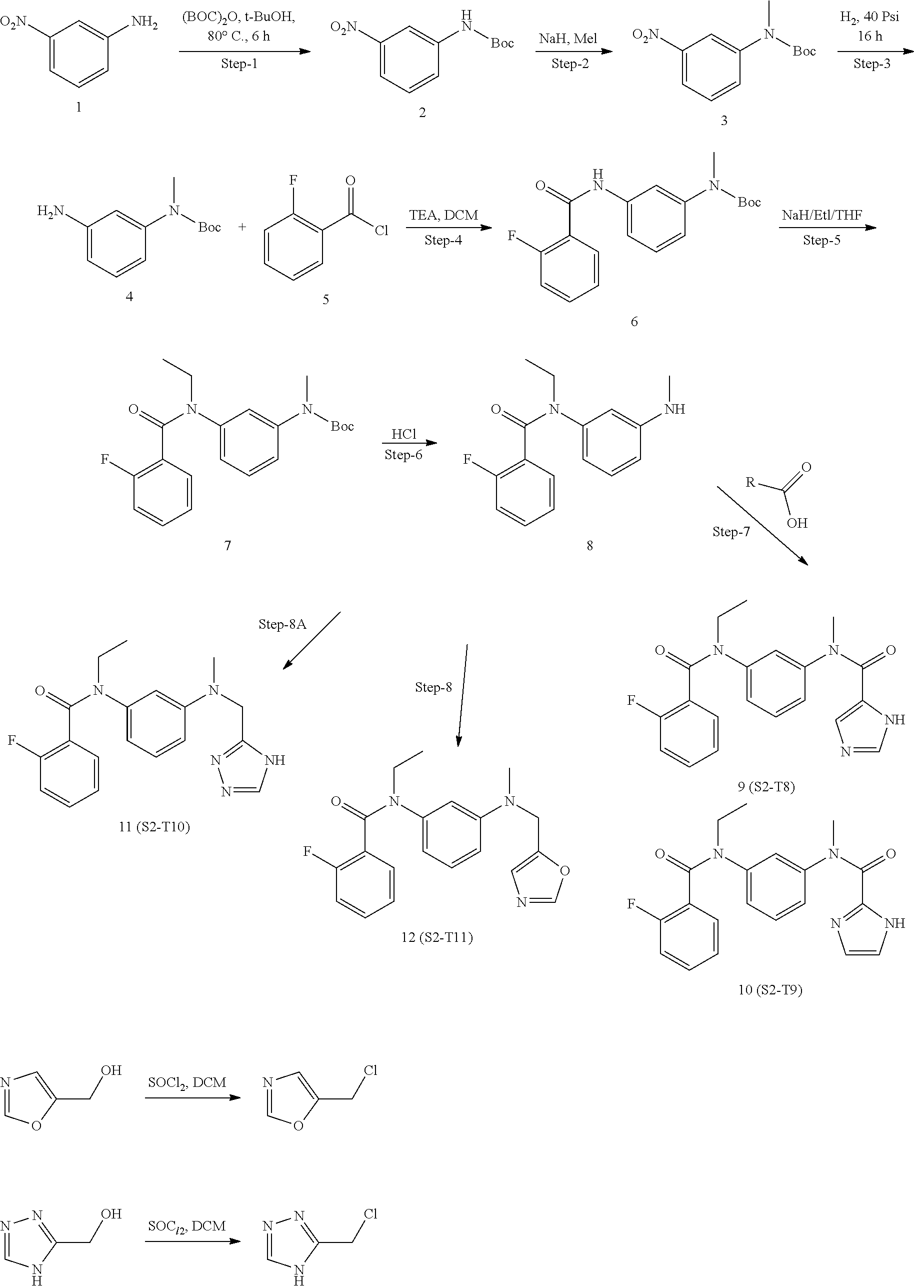

Step-1: Synthesis of tert-butyl N-(3-nitrophenyl)carbamate (CR652-18239-29-1-P)

To a stirred solution of 3-nitroaniline (10.0 g, 72.39 mmol) in t-BuOH (15 ml) was added Boc-anhydride (21.066 ml, 218.25 mmol). The resulting suspension was stirred at 80° C. for 16 hours. The solvent was removed under vacuum and the residue was washed with n-pentane to afford t-butyl N-(3-nitrophenyl)carbamate as light yellow solid (9.0 g, 52.19% yield).

Step-2: Synthesis of tert-butyl N-methyl-N-(3-nitrophenyl)carbamate (CR652-18239-46-1-P)

NaH (60% suspension in oil, 0.504 g, 8.403 mmol) was taken in dry DMF under nitrogen atmosphere & cooled to 0° C. To it was added t-butyl N-(3-nitrophenyl) carbamate (5.0 g, 20.202 mmol) and the reaction mixture was stirred for 30 minutes at 0° C. Then methyl iodide (5.5 ml, 30.606 mmol) was added slowly to the reaction mixture and allowed to warm to 23° C. over 16 hours. The reaction mixture was quenched with saturated NH₄Cl solution and extracted with ethyl-acetate (2×250 ml). Combined organic part was dried over anhydrous sodium sulphate, filtered and concentrated in vacuo and triturated with ether to afford tert-butyl N-methyl-N-(3-nitrophenyl)carbamate (5.2 g, 90.57% yield) as a light yellow solid.

Step-3: Synthesis of tert-butyl N-(3-aminophenyl)-N-methylcarbamate (CR652-18239-48-1-P)

A parr flask was charged with a degassed solution of tert-butyl N-methyl-N-(3-nitrophenyl)carbamate (5.1 g, 4.762 mmol) in methanol (60 ml) followed by addition of 10% Pd—C (˜50% moist, 2.5 g). The reaction was hydrogenated at 40 psi for 16 hours. The reaction mass was filtered through sintered funnel using a pad of celite, washed repeatedly with methanol and concentrated under reduce pressure to afford tert-butyl N-(3-aminophenyl)-N-methylcarbamate as brown solid (3.2 g, 75.58% yield) which was used as such for the next step without any further purification.

Step-4: Synthesis of tert-butyl N-{3-[(2-fluorobenzene)amido]phenyl}-N-methyl carbamate (CR652-18239-63-1-C)

To a stirred solution of tert-butyl N-(3-aminophenyl)-N-methylcarbamate (3.0 g, 13.513 mmol) in DCM (25 ml), TEA (5.76 ml, 40.539 mmol) was added. The reaction mixture was stirred at 0° C. for 30 minutes then 2-fluorobenzoyl chloride (1.606 ml, 13.513 mmol) was added and the reaction mixture was allowed to warm to 23° C. over 16 h. The reaction mixture was diluted with DCM (20 ml) and washed with saturated NaHCO₃ solution. The aqeuous part was re-extracted with DCM (2×15 ml) and combined organic part was dried over anhydrous sodium sulphate, filtered and concentrated in vacuo to afford a brown gum. Purification by combiflash column chromatography (silica column, ethyl acetate 0% to 50% gradient of a solution) afforded N-{3-[(2-fluorobenzene)amido]phenyl}-N-methylcarbamate (2.5 g, 55% yield) as brown solid.

Step-5: Synthesis of tert-butyl N-{3-[N-ethyl(2-fluorobenzene)amido]phenyl}-N-methylcarbamate (CR652-18239-68-1-P)

To a stirred solution of N-{3-[(2-fluorobenzene)amido]phenyl}-N-methylcarbamate (2.5 g, 7.267 mmol) in THF (10 ml), NaH (60% suspension in mineral oil, 700 mg, 14.534 mmol) was added slowly and the mixture was stirred at 0° C. for 45 minutes. Ethyl Iodide (1.16 ml, 14.534 mmol) was added and the reaction stirred at 0° C. for 30 minutes and at 23° C. for 16 h. The reaction mixture was quenched with saturated NH₄Cl solution and extracted with ethyl-acetate (50 ml). The combined organic layer was washed with brine, dried over anhydrous Na₂SO₄, filtered and concentrated in vacuo to afford tert-butyl N-{3-[N-ethyl(2-fluorobenzene)amido]phenyl}-N-methylcarbamate (220 mg, 81.49% yield) as brown gum.

Step-6: Synthesis of N-ethyl-2-fluoro-N-(3-(methylamino)phenyl)benzamide (CR652-18239-88-1-R1)

To a stirred solution of tert-butyl N-{3-[N-ethyl(2-fluorobenzene)amido]phenyl}-N-methylcarbamate (500 mg, 1.344 mmol) in dioxane (2 ml) at ice cold condition was added HCl-dioxane (4 M, 3 ml) and the reaction mixture was stirred at 23° C. for 16 h. The reaction was neutralized with saturated NaHCO₃ solution and extracted with ethyl acetate to afford N-ethyl-2-fluoro-N-[3-(methylamino)phenyl]benzamide as a free base (320 mg, 87.43% yield).

General Procedure for Amidation with N-ethyl-2-fluoro-N-(3(methylamino)phenyl)benzamide (Cpd-8)

To a stirred solution of 1H-imidazole-5-carboxylic acid/1H-imidazole-2-carboxylic acid (0.551 mmol, 1 eq) and N-ethyl-2-fluoro-N-(3(methylamino)phenyl)benzamide (0.551 mmol, 1.0 eq) in moist DMF (0.1 ml) were added T3P (˜50% in ethyl acetate, 1.654 mmol, 3 eq) and pyridine (3.309 mmol, 6.0 eq) and heated at 70° C. for 3 hours. The reaction mixture was diluted with ethyl-acetate (20 ml) and washed with water and saturated NaHCO₃ solution. The organic layer was washed with brine, dried over anhydrous Na₂SO₄, filtered and concentrated in vacuo to afford crude material. The crude material was purified by prep-HPLC (using NH₄HCO₃ buffer) followed by lyophilization to afford desired compound.

S2-T8: N-[3-[ethyl-(2-fluorobenzoyl)amino]phenyl]-N-methyl-1H-imidazole-5-carboxamide (Off white solid, 38.49 mg, 19.05%)

¹H NMR (400 MHz, DMSO-d₆ at 373K) δ 12.21 (bs, 1H), 7.49 (s, 1H), 7.27-7.31 (m, 3H) 6.99-7.11 (m, 5H), 5.61 (bs, 1H), 3.84 (s, 2H), 3.21 (s, 3H), 1.09 (t, 3H, J=8 Hz).

LCMS Analytical Method B: rt=2.12 minutes, m/z 365.03 [M−H]⁺.

S2-T9: N-[3-[ethyl-(2-fluorobenzoyl)amino]phenyl]-N-methyl-1H-imidazole-2-carboxamide (Off white solid, 45.48 mg, 22.51%)

¹H NMR (400 MHz, DMSO-d₆ at 373K) δ 12.20 (bs, 1H), 7.20-7.29 (m, 3H), 6.96-7.08 (m, 5H), 3.79-3.81 (m, 2H), 3.41 (s, 3H), 1.11 (t, 3H, J=8 Hz).

LCMS Analytical Method B: rt=2.42 minutes, m/z 365.03 [M−H]⁺.

### General Procedure for Chlorination of Substituted Hydroxymethanol Oxazole/Triazole

To a stirred solution of oxazol-5-ylmethanol (430 mg, 4.34 mmol, 1.0 equiv) in DCM (10 mL) was added thionyl chloride (0.48 ml, 6.51 mmol, 1.5 equiv) dropwise over 5 minutes. The reaction was stirred at reflux for 3 hours and then was neutralized with sat. Na₂CO₃ and extracted with ethyl acetate (3×50 ml). The combined organics were passed through a hydrophobic frit and concentrated in vacuo to afford 5-(chloromethyl)-oxazole (448 mg, 3.81 mmol, 88% yield) as a yellow oil, which was used without further purification.

To a stirred solution of 3-(hydroxymethyl)-4H-1,2,4-triazole·hydrochloride (500 mg, 3.526 mmol) in ice cooled condition, thionyl chloride (5 ml) was added dropwise and stirred at 90° C. for 3 hours. The reaction mixture was concentrated in vacuo followed by trituration with ether-pentane (1:1) to afford 3-(chloromethyl)-4H-1,2,4-triazole·hydrochloride (380 mg, 3.75 mmol, 85% yield) as off white solid, which was used without further purification.

General Procedure for N-alkylation on N-ethyl-2-fluoro-N-(3(methylamino)phenyl)benzamide (Cpd-8)

To a stirred solution of N-ethyl-2-fluoro-N-(3-(methylamino)phenyl)benzamide (0.551 mmol, 1.0 eq) in dry acetonitrile (7 ml) was added Cs₂CO₃ (1.103 mmol, 2.0 eq) followed by 5-(chloromethyl)-oxazole/3-(chloromethyl)-4H-1,2,4-triazole (1.103 mmol, 2.0 eq) in acetonitrile with cat KI. The reaction mass was heated at 80° C. for 3 h. The reaction mass was filtered and acetonitrile was evaporated. The crude material was purified by prep-HPLC (using NH₄HCO₃ buffer) followed by lyophiliszation to afford desired compound.

S2-T10: N-ethyl-2-fluoro-N-[3-[methyl (4H-1,2,4-triazol-3-ylmethyl)amino]phenyl]benzamide (Light yellow solid, 91.16 mg, 35.08%)

¹H NMR (400 MHz, DMSO-d₆ at 373K) δ 13.46 (bs, 1H), 8.15 (s, 1H), 7.19-7.22 (m, 2H), 6.95-7.07 (m, 3H), 6.57-6.62 (m, 2H), 6.39-6.41 (m, 1H), 4.44 (s, 2H), 3.78-3.80 (m, 2H), 2.93 (s, 3H), 1.11 (bs, 3H).

LCMS Analytical Method B: rt=2.27 minutes, m/z 354.2 [M+H]⁺.

S2-T11: N-ethyl-2-fluoro-N-[3-[methyl(oxazol-5-ylmethyl)amino]phenyl]benzamide (Off White gum, 53.72 mg, 25.57%)

¹H NMR (400 MHz, DMSO-d₆ at 373K) δ 8.14 (s, 1H), 7.12-7.27 (m, 2H), 6.95-7.05 (m, 3H), 6.86 (s, 1H), 6.46-6.63 (m, 2H), 4.48 (s, 2H), 3.82 (qt, 2H, J=8 Hz), 2.84 (s, 3H), 1.13 (t, 3H, J=8 Hz).

LCMS Analytical Method B: rt=2.76 minutes, m/z 354.19 [M+H]⁺.

### S2-T12

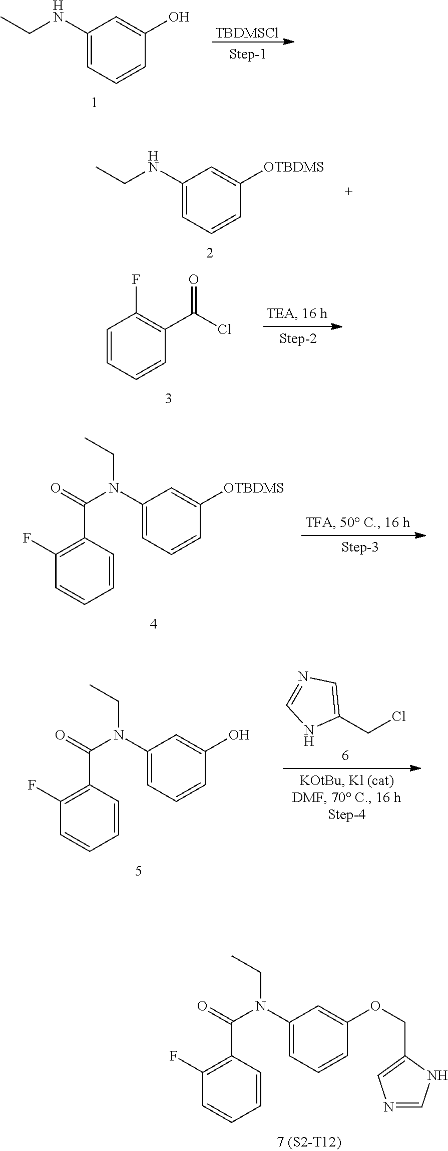

Step-1: Synthesis of 3-[(tert-butyldimethylsilyl)oxy]-N-ethylaniline (CR652-18239-142-1-R)

To a stirred solution of 3-(ethylamino) phenol (600 mg, 4.38 mmol, 1.0 eq) in DCM (10 ml) and THF (10 ml) at 0° C., imidazole (595.62 mg, 8.759 mmol, 2.0 eq) was added portion wise then TBDMS-Cl (990.131 mg, 6.569 mmol, 1.5 eq) was added at 0° C. dropwise and stirred at 23° C. for 16 hours. The reaction mixture was quenched with NH₄Cl solution and extracted with DCM and the organic layer washed with brine and concentrate to afford 3-[(tert-butyldimethylsilyl)oxy]-N-ethylaniline (980 mg, 88.99% yield) as a brownish liquid.

Step-2: Synthesis of N-{3-[(tert-butyldimethylsilyl)oxy]phenyl}-N-ethyl-2-fluoro benzamide (CR652-18239-143-1-P)

A solution of 3-[(tert-butyldimethylsilyl)oxy]-N-ethylaniline (500 mg, 1.989 mmol) in DCM (6 ml) was stirred at 0° C. for 5 minutes. Then TEA (1.15 ml, 7.955 mmol, 4.0 eq) was added dropwise at ice cooled condition. Then 2-fluorobenzoyl chloride (0.236 ml, 1.989 mmol, 1.0 eq) dissolved in DCM (6 ml) and then added to reaction mixture dropwise at 0° C. The reaction mixture was stirred at 23° C. for 16 hours. The reaction mixture was quenched with ice cooled water and extracted with ethyl-acetate (2×100 ml), washed with brine solution, dried over anhydrous Na₂SO₄, filtered and concentrated in vacuo to afford N-{3-[(tert-butyl dimethyl silyl)oxy]phenyl}-N-ethyl-2-fluorobenzamide (660 mg, 88.85%).

Step-3: Synthesis of N-ethyl-2-fluoro-N-(3-hydroxyphenyl)benzamide (CR652-18239-145-1-P)

To a stirred solution of N-{3-[(tert-butyl dimethyl silyl)oxy]phenyl}-N-ethyl-2-fluoro benzamide (600 mg, 1.536 mmol, 1.0 eq) in DCM (4 ml), TFA (1.2 ml, 15.362 mmol, 10.0 eq) was added dropwise at 0° C. and refluxed at 70° C. for 16 hours. The reaction mass was concentrated to remove excess TFA and quenched with aqueous NaHCO₃ solution and extracted with DCM (2×50 ml). The combined organic layer was washed with brine, dried over anhydrous Na₂SO₄ and concentrated in vacuo to afford N-ethyl-2-fluoro-N-(3-hydroxyphenyl)benzamide (300 mg, 71.93%) as a brown liquid.

Step-4: Synthesis of N-ethyl-2-fluoro-N-[3-(1H-imidazol-5-ylmethoxy)phenyl]benzamide (CR652-18239-156-1-P)

To a stirred solution of N-ethyl-2-fluoro-N-(3-hydroxyphenyl)benzamide (250 mg, 0.962 mmol) in dry DMF (10 ml) was added potassium-t-butoxide (1 M solution in THF) (3.28 g, 2.402 mmol, 2.5 eq) followed by 4-(chloromethyl)-1H-imidazole·hydrochloride (103 mg, 0.673 mmol, 0.7 eq) in acetonitrile with cat KI. The reaction mass was heated at 80° C. for 16 hours. The reaction mixture was filtered and concentrated. Ethyl acetate was added (75 ml) and washed with water (100 ml). The aqueous part was further extracted with ethyl acetate (2×100 ml). The combined organic part was washed with brine, dried over anhydrous sodium sulphate, filtered and solvent was evaporated in vacuo to afford crude product as an orange oil. The crude material was purified by prep-HPLC (using NH₄HCO₃ buffer) followed by lyophiliszation to afford N-ethyl-2-fluoro-N-[3-(1H-imidazol-5-ylmethoxy)phenyl]benzamide as off white gum.

S2-T12: N-ethyl-2-fluoro-N-[3-(1H-imidazol-5-ylmethoxy)phenyl]benzamide (Off white gum, 11.32 mg, 8.67%)

¹H NMR (400 MHz, DMSO-d₆ at 373K) δ 11.78 (bs, 1H), 7.56 (s, 1H), 7.25-7.30 (m, 2H), 6.98-7.14 (m, 3H), 6.57-6.83 (m, 4H), 4.87 (s, 2H), 3.77-3.86 (m, 2H), 1.14 (t, 3H, J=8 Hz).

LCMS Analytical Method B: rt=2.32 minutes, m/z 340.2 [M+H]⁺.

### Results and Discussion

Around 350,000 drug-like molecules were screened through a primary screen assay to identify any hits which would inhibit 17β-HSD10. After further validation using orthogonal assays, dose response assays and assessing physicochemical properties a cluster of five molecules featuring an imidazole benzamide as the key structural feature and a singleton molecule were identified as starting points for obtaining lead molecules.

Further analogues were synthesised and tested through various screening assays. Primary screening results are shown in Table 1. Further analogue series were synthesised focussing on optimising each area of the molecule to increase target engagement, potency and simultaneously improving the physicochemical properties.

TABLE 1
Primary screening plC₅₀ values for the resynthesized HTS hit compounds.

| | | Compound | |
|---|---|---|---|
| Compound Origin | Structure | identifier | ABAD plC₅₀ |
| Cluster | 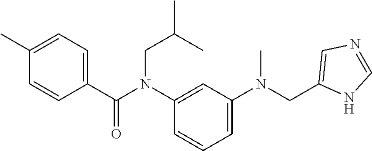 [SMILES: Cc1ccc(C(=O)N(CC(C)C)c2cccc(N(C)Cc3cnc[nH]3)c2)cc1] | ESC1002032 | 6.6 |
| Cluster | 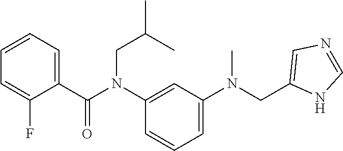 [SMILES: CC(C)CN(C(=O)c1ccccc1F)c1cccc(N(C)Cc2cnc[nH]2)c1] | ESC1002033 | 6.7 |
| Cluster | 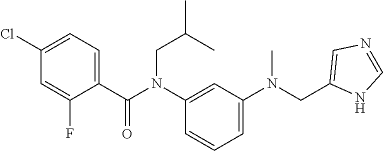 [SMILES: CC(C)CN(C(=O)c1ccc(Cl)cc1F)c1cccc(N(C)Cc2cnc[nH]2)c1] | ESC1002042 | 6.5 |
| Cluster | 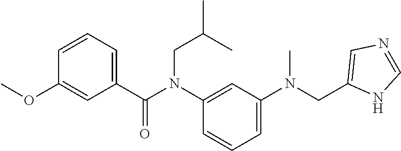 [SMILES: COc1cccc(C(=O)N(CC(C)C)c2cccc(N(C)Cc3cnc[nH]3)c2)c1] | ESC1002038 | 5.7 |
| Cluster | 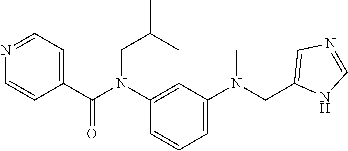 [SMILES: CC(C)CN(C(=O)c1ccncc1)c1cccc(N(C)Cc2cnc[nH]2)c1] | ESC1002034 | 5.8 |
| Singleton | 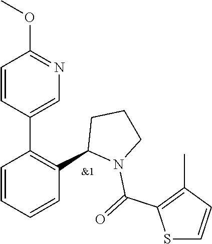 [SMILES: COc1ccc(-c2ccccc2[C@H]2CCCN2C(=O)c2sccc2C)cn1] | ESC1002082 | 6.1 |

The physicochemical properties of the resynthesized hits are detailed in Table 2. The structures of the compounds may be found in Table 6. Overall the compounds are lead-like, although the lipophilicities of some examples are higher than optimal. The physicochemical properties lie within the idealised limits required for brain exposure (see Lipinski, C. A., Lombardo, F. and Dominy, B. W., Adv. Drug Deliv. Rev. 1997, 23, 3-25.

TABLE 2
Physicochemical properties for the resynthesized HTS
compounds. Where MW = molecular weight (Da),
ALogP = atom additive partition coefficient, LogD =
distribution coefficient, HBA = hydrogen bond acceptor,
HBD = hydrogen bond donor, LipE = lipophilic efficiency.

| Identifier | MW | ALogP | LogD | TPSA | HBA | HBD | LipE |
|---|---|---|---|---|---|---|---|
| Optimal | <500 | <5 | <5 | <90 | <7 | <3 | pIC50- |
| | | | | | | | LogP |
| ESC1002033 | 380 | 3.93 | 4.37 | 52 | 3 | 1 | 2.33 |
| ESC1002032 | 376 | 4.21 | 4.65 | 52 | 3 | 1 | 1.95 |
| ESC1002042 | 415 | 4.6 | 5.04 | 52 | 3 | 1 | 1.46 |
| ESC1002034 | 363 | 2.58 | 3.02 | 65 | 4 | 1 | 2.78 |
| ESC1002038 | 392 | 3.71 | 4.15 | 61 | 4 | 1 | 1.55 |
| ESC1002082 | 378 | 4.65 | 4.65 | 71 | 3 | 0 | 1.45 |

### Cluster Chemical Modifications

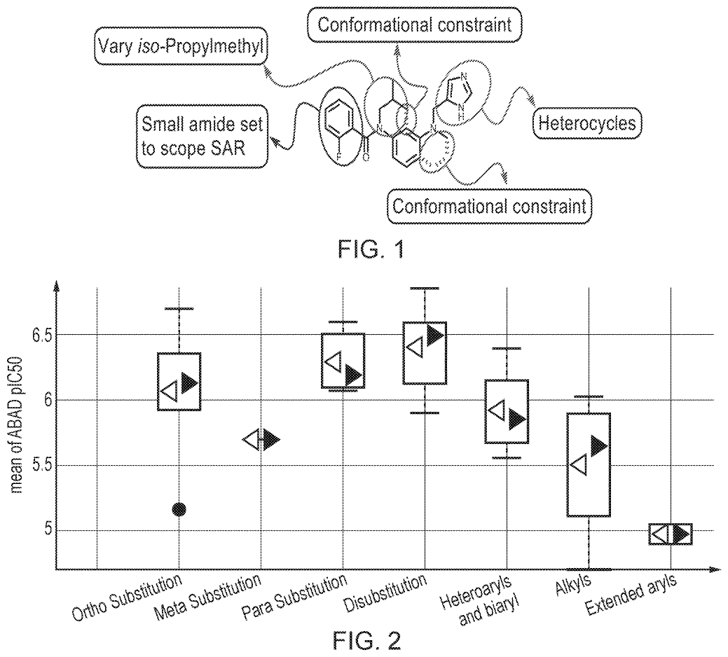

The cluster analogues identified by the primary screen were altered on the amide side of the molecule and initial structure activity relationships (SAR) within the hits suggested that both ortho and/or para substitution with an electron withdrawing group was favourable for potency. Initial analogues focussed on exploring this region further with a small number of additional aryl amides. As well as these changes, initial SAR exploration around the other parts of the hit were also planned to establish some initial SAR around these regions. Chemical alterations are shown in FIG. 1.

Utilising the primary screen and dose response assays, results indicated that the replacement of the iso-propylmethyl results in some loss of potency with relatively subtle changes (Table 6). As well as these subtle changes, SAR would suggest that heteroatoms e.g. ESC1002334 and heteroaryl rings reduce potency e.g. ESC1002516.

Expansion of the SAR around the initial hits indicate that ortho and para substitution of the terminal phenyl ring (e.g. fluorophenyl of FIG. 1) with both electron donating and withdrawing groups is well tolerated. Di-substitution was also well tolerated and may give rise to slightly improved inhibition of ABAD. Replacement of the terminal phenyl ring with an alkyl was also investigated. Although replacement with cyclohexyl (compound ESC1002166) resulted in a less potent compound, this compound may have improved physicochemical and drug metabolism and pharmacokinetic properties. Extending the terminal phenyl ring outward with insertion of methylenes gave rise to losses in potency. The box plot in FIG. 2 summarises the compound type versus 17β-HSD10 activity.

It was also established that the appropriate positioning of a H-bond donor effects 17β-HSD10 activity, c.f. ESC 1002199 and ESC1002033, and ESC1002205 and ESC1002206 (Table 6). Furthermore, comparing the imidazole/pyrazole pair, ESC1002033 and ESC1002205, suggests that an H-bond acceptor (likely to be protonatable) also effects activity. When comparing ESC1002033 and ESC1002208, the thiophene and pyrazole will be un-ionised at physiological pH. In addition, there is some space around the terminal imidazole group (see FIG. 1) for further functionalisation with both methyl substituted compounds, ESC1002203 and ESC1002204 having only slightly reduced ABAD inhibition levels in comparison to ESC1002033. Expansion of the terminal imidazole to a 6-membered ring is also tolerated. It was also noted that when conformational constraints were implemented to lock the molecule into its bioactive conformation (see FIG. 1 and compound ESC1002081 of Table 6), that potency was abolished.

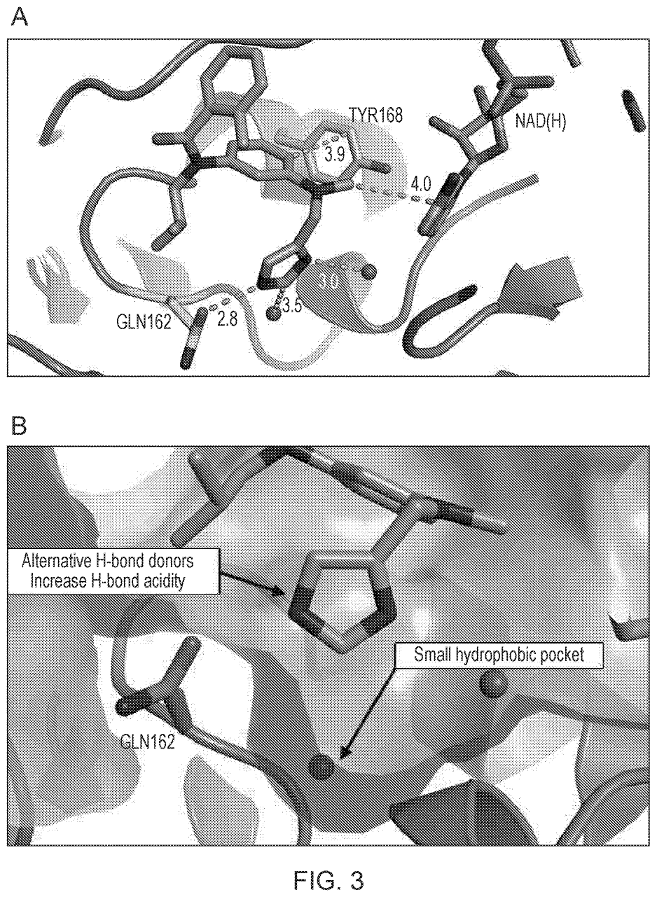

Following the solving of the crystal structure of ESC1002033 within 17β-HSD10 (discussed in more detail later), key interactions between 17β-HSD10 and ESC1002033 were found to include an H-bond from the imidazole to GLN162 and possibly also a H-bond to a well-ordered water molecule (FIG. 3a). In addition, an edge to face interaction between the central aryl ring and TYR168 of the catalytic triad is evident. With the crystal structure available, new opportunities presented themselves to develop further cluster analogues with increased potency. Specifically, a further set of analogues were prepared that maintained a H-bond donor in an equivalent position or increased the acidity of the donor itself.

The crystal structure revealed a small hydrophobic pocket close to the imidazole with a well-ordered water molecule present (FIG. 3b). There was therefore an opportunity to fill the pocket and potentially displace the water molecule to gain potency. In the initial heterocyclic replacement set, the 2-methylsubstituted imidazole, ESC1002203 had already been prepared and was approximately 3-fold less potent than ESC1002033 (Table 6). Increasing the size of the 2-substituent to an iso-propyl, ESC1002633, resulted in further losses in potency. The slightly smaller and more sp²-like cyclo-propyl was better tolerated than its iso-propyl congener (Table 6).

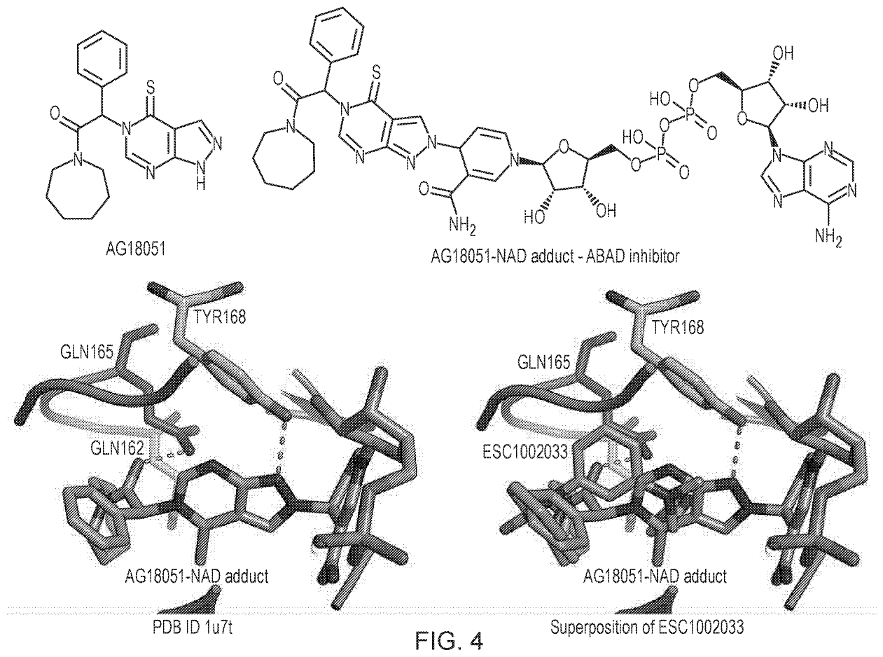

The crystal structure of ESC1002033 was superimposed with another crystal structure. At the time of experimentation, the only other reported crystal structure of a ligand bound to 17β-HSD10 is AG18051 (C. R. Kissinger et al., J. Mol. Biol., 2004, 342 (3), 943-952). This ligand forms a covalent adduct with NADH, catalysed by 17β-HSD10, resulting in a highly potent inhibitor of the protein. Key interactions formed by the protein-ligand complex are a H-bond donor to TYR168 and a H-bond to GLN162. Superposition of ESC1002033 onto the AG18051 crystal structure (FIG. 4) indicates that the TYR168 H-bond is not engaged by the ESC1002033 ligand. Analogues were prepared to try and exploit this interaction.

Attempts to target the H-bond of TYR168 resulted in losses in potency. Interestingly, the unsubstituted imidazole analogue ESC1002631, which was prepared as an intermediate, illustrates the importance of methyl substitution at imidazole (Table 6).

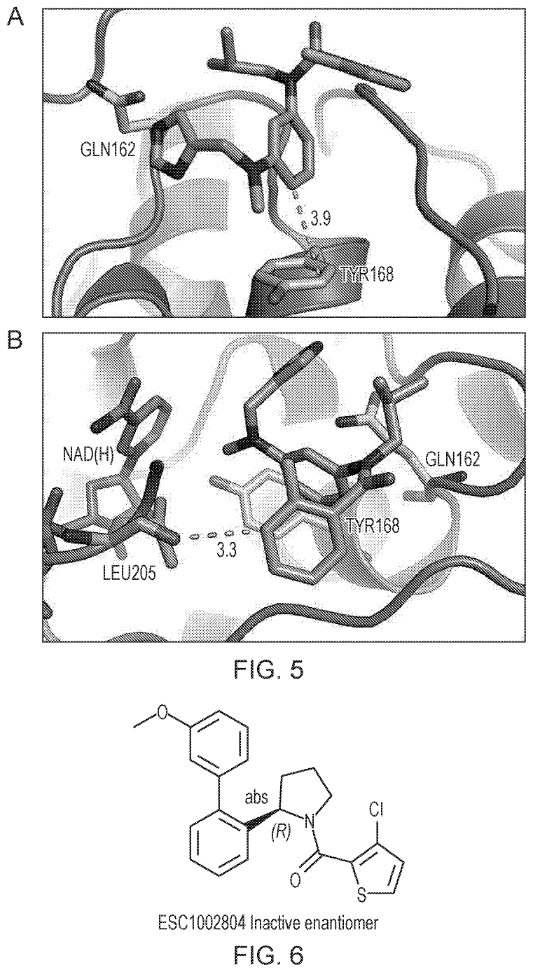

A potentially key interaction identified in the ligand bound crystal structure of ESC1002033 was the edge-to-face π-interaction between TYR168 and the central aryl ring (FIG. 5). This interaction can be strengthened by making the interacting hydrogen atom(s) more acidic. Therefore, a set of heteroaromatic replacements were prepared which, due to their electronic nature, should increase the acidity of the interacting hydrogen. However, no improvement in potency was observed.

The last point of inquiry within this series was a potential H-bond interaction with the backbone carbonyl of LEU205. As detailed earlier in the report, an extensive array of both substituents and substitution patterns had already been prepared and tested. The overall conclusion from that work was that both 2-substitution and 2,6-disubstitution resulted in improved potencies, the original QHL hit ESC1002033 and ESC1002456 were identified as the most potent inhibitors within the series. Fluorine is a strongly electronegative group and will polarize the adjacent proton on the aromatic ring, making it more acidic. This would then be set-up for a weak H-bond between the Aryl-H and the carbonyl of LEU205 and indeed the distance between the Aryl-H and the carbonyl is consistent with a weak H-bond. To explore this further several analogues were prepared that either placed a H-bond donor in this position or a polarised hydrogen of a heterocycle. However, these modifications did not improve potency.

### Cluster Chemical Modification Summary

To summarise, an extensive range of analogues were prepared and tested to explore the SAR around the cluster hit series. In some cases, it was found that replacement of key features in ESC 1002033 resulted in potency loses.

### Singleton Chemical Modifications

The singleton hit ESC1002082 was identified as a moderately potent hit from the primary screen (Table 1). The physicochemical properties were consistent with a molecule capable of entering the central nervous system (CNS), although its lipophilicity was higher than optimum and its LipE was low (Table 2). The ESC1002082 compound contains a single chiral centre and was registered in the database as a racemate. The initial plan was focussed on ascertaining whether the stereochemical configuration of the chiral centre is important for activity and if any SAR trends emerge with simple deletion analogues and amide changes.

### Stereochemistry

A chiral synthetic route or intermediate was not readily available for the preparation of enantiomerically pure material. Therefore, to obtain and test the two constituent enantiomers a semi-preparative chiral SFC method was developed that allowed efficient separation of the enantiomers. The enantiomers were tested in a 17β-HSD10 dose response assay (results are shown in Table 3).

TABLE 3
ESC1002082 enantiomers and their pIC50 values

| Identifier | Enantiomer | 17β-HSD10 pIC50 |
|---|---|---|
| ESC1002082 | Racemic | 6.1 |
| ESC1002161 | Enantiomer 1 | <4.7 (7) |
| ESC1002162 | Enantiomer 2 | 6.31 |

It was found that much of the 17β-HSD10 inhibitory activity resided in a single enantiomer. This not only provided confirmation that the compound is a true inhibitor of 17β-HSD10 activity, but also meant that access to a closely related inactive analogue (often useful in more complex assays) should be readily available. Given that the separation of enantiomers by SFC is not trivial, the analogue plan focussed on initial testing of the racemates with key compounds being separated by SFC for further testing. We have subsequently identified that the active enantiomer has an S configuration

### Deletion Analogues and Amide Changes

The results of the deletion and amide changes indicated that the deletion of the nitrogen from the pyridone of ESC1002082 to the 4-methoxybenzene, ESC1002089, resulted in a just under 3-fold improvement in activity which was boosted further on separation and testing of the single enantiomer, ESC1002332 (Table 6). Deletion of the methoxy was not significantly detrimental to activity, ESC1002090. In contrast to the positive SAR observed with these modifications, changes to the amide side of the molecule were less well-tolerated. Replacement of the 2-methyl thiophene of ESC1002082 for 2-fluorobenzene gave a drop in potency and substitution with 4-methylbenzene resulted in complete ablation of activity. These replacements suggested that the presence of a thiophene and ortho-substitution are potency drivers in this series. The potency enhancements observed with the deletion analogues focussed initial SAR expansion efforts to aryl ring replacements, with the aim of further potency gains. In addition, incorporation of more polar heterocycles and substituents were explored to reduce lipophilicity.

In this series, a variety of substituents and substitution patterns are well tolerated with most compound modifications retaining activity levels within a log unit of the original hit, ESC1002082. Less favoured groups are polar electron withdrawing groups e.g. the sulfones ESC1002322 and ESC1002319 (results Table 6). On changing the position of the methoxy group of ESC1002089 around the benzene ring, it was found that the potency of the ortho-methoxy analogue was similar to ESC1002089 (para-methoxy), and the potency of the meta-methoxy analogue (ESC1002338) was greater than ESC1002089, exhibiting an IC₅₀ of 191 nM. An analogue comprising both ortho-methoxy and meta-methoxy (ESC1002326) did not lead to additive effects (Table 6). Heterocyclic changes were also reasonably well tolerated. The pyrazole derivative ESC1002424 had comparable affinity to the QHL hit (ESC1002082) but improved physicochemical properties (ESC1002082 LipE 1.45 vs ESC1002424 LipE 2.1).

Having expanded the aryl ring SAR and identified ESC1002338 as a more potent 17β-HSD10 inhibitor, the next area of analogue expansion was around the aryl amide. SAR around the amide was found to be limited. Altering the position of the methyl substituent on the thiophene group resulted in a loss of activity, c.f. ESC1002338 vs. ESC1002428 and ESC1002426 (Table 6). In addition, the type of substituent also influenced potency, with greater potencies achieved with small apolar substituents. Of note is the 3-chloro substituted thiophene, ESC1002432 (racemic) and ESC1002606 (chiral), which gave over 3-fold improvement in activity with an IC₅₀ of approximately 100 nM. Some simple heterocyclic changes reduced the potency of the resultant singleton: the bioisosteric equivalents of the thiophene, ESC1002429 and ESC1002430 were significantly less active. Replacing the thiophenyl with 4-chloropyrazolyl (ESC1002626 (racemic) and ESC1002757 (chiral)) resulted in similar activity levels to ESC1002606 but also had improved lipophilicity with a 1-unit improvement in LipE (ESC1002606 LipE 1.52 vs. ESC1002757 LipE 2.76). Direct replacement of the 5-membered heterocycle with its benzene isostere was not tolerated (ESC1002434) nor were cycloalkyl replacements. Finally, the importance of the carbonyl amide was confirmed with the synthesis of ESC1002695, which lost all activity in comparison to ESC1002431. Having identified some improved affinity and LipE ligands in this phase of the project, the distal aryl ring was revisited with these newly identified higher potency substituents with the aim of improving both affinity and physicochemical properties further.

It was shown that increasing the electron richness of the aromatic ring (by introduction of the methoxy groups) resulted in a small decrease in potency as did further substitution in the para position. Lower lipophilicity heterocycles were tolerated but resulted in lower activity. More successful was demethylation to the corresponding phenol which gave a significant improvement in potency in comparison to its methoxy counterpart and resulted in the identification of ESC1002755 with an IC₅₀=19 nM the most potent compound identified in the IHL work. Interestingly, both bioisosteric equivalents of this compound the pyridone (ESC1002697) and indazole (ESC1002702) were less active. Fluorination of the aromatic ring was well tolerated, but resulted in no further potency improvement, but may serve to protect the electron rich aromatic ring from further oxidative metabolism.

### Determining the Absolute Conformation of the Enantiomer

In order to determine the absolute configuration of the active enantiomer, access to the single enantiomer was achieved via chiral separation of the racemate by SFC. To determine the configuration, a homochiral intermediate was purchased (Sigma Aldrich) and reacted to give ESC1002804 with a known configuration. This compound (shown in FIG. 6) which has R stereochemistry was tested in a 17β-HSD10 inhibition assay and found to be inactive, pIC₅₀<4.7. Thus, by corollary, the active enantiomer of the series is S.

### X-Ray Crystal Structure of ESC1002421

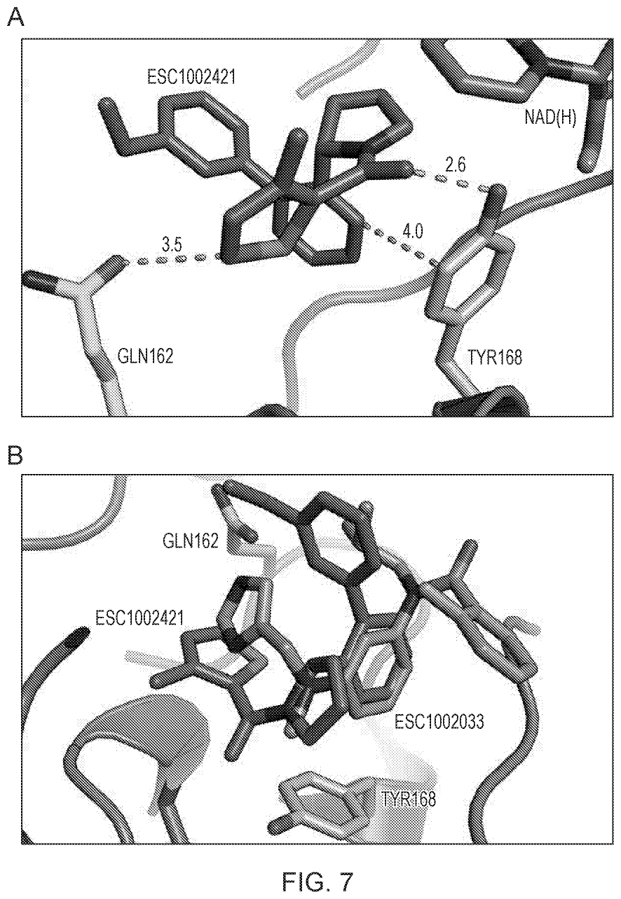

The ligand bound crystal structure of ESC1002421 is illustrated in FIG. 7a. Key interactions include an edge-to-face interaction with TYR168, like the cluster series, but there is also an additional H-bond interaction from the amide carbonyl to the catalytic triad phenol TYR168. Furthermore, the modelled structure is consistent with the active enantiomer being in the S-configuration. In FIG. 7b, right panel, a superposition of ESC 1002421 and ESC10022033 is presented.

### Singleton Chemical Modification Summary:

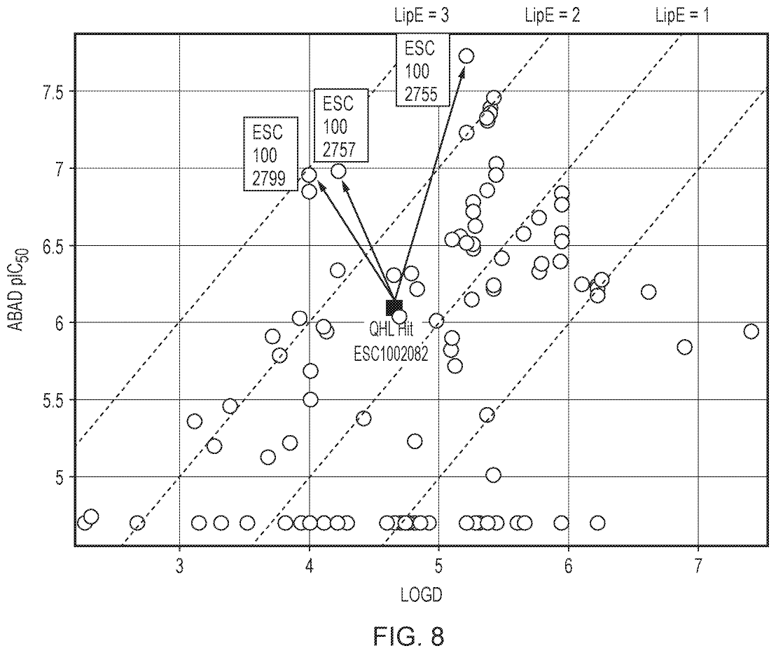

Using ESC1002082 as a starting point, an extensive collection of hits have been synthesised, principally by amendment around the distal aryl ring and the amide. Significant improvements in potency were achieved and several sub-100 nM ligands were identified, the most potent of which is ESC1002755 with an IC₅₀=19 nM an overall 40-fold improvement in potency (Table 6). In addition to the significant potency gains, several ligands with improved physicochemical properties were also identified, such as ESC1002799 (LipE=2.96) and ESC1002757 (LipE=2.76) with not only improved potency but also improved lipophilicity. Finally, through chemical synthesis, the eutomer was determined to have S-stereoconfiguration and is consistent with the solved crystal structures of the series. A summary of lipophilicity vs potency improvements is presented in FIG. 8.

### Thermal Shift Analysis Results

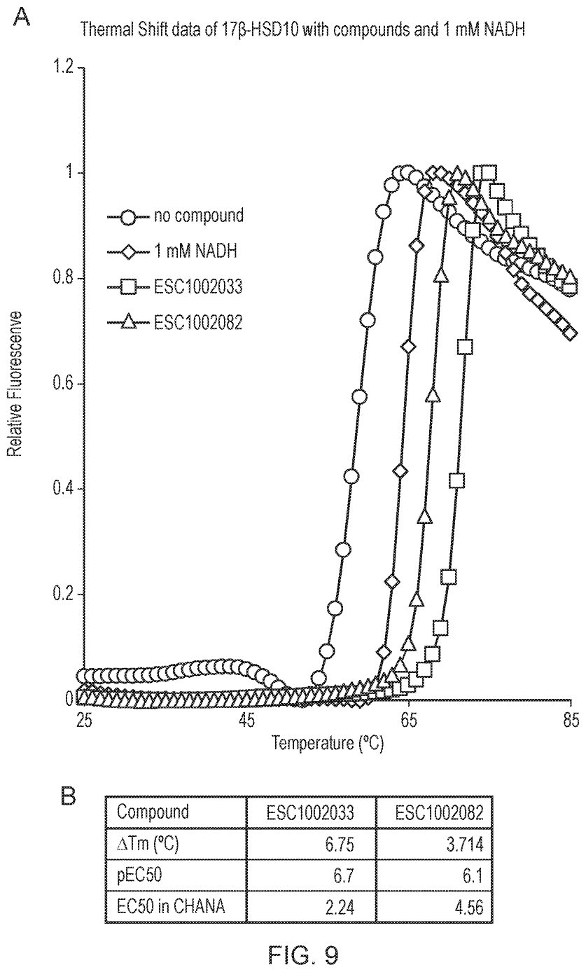

Results of a thermal shift assay (TSA) showed that some of the QHL compounds stabilised 17β-HSD10 in the presence of NADH, providing strong evidence of target engagement. The ΔTm for one compound from each series is presented in FIG. 9 with a representative derivative plot of ESC1002033 and ESC1002082 in FIG. 9a. A ΔTₘ was analysed for each compound at 20 μM compound concentration and 1 mM NADH and FIG. 9b shows the correlation between pIC₅₀, the cellular assay and ΔTₘ. None of the compounds could improve on the ΔTₘ=6.75° C. exhibited by compound ESC1002033 with most ΔTₘ shifts around 1-4° C., therefore still displaying good target engagement. However, some of the compounds showed a degree of autofluorescence and as such are unsuitable for analysis using this technique.

### Mechanism of Action Results

Two compounds, ESC1002033 and ESC1002082, representative of the two different clusters were investigated for their mechanism of action against the substrates, NADH and AcetoAcetyl CoA (AcAc). The ESC inhibitors were tested as 12-point 1 in 1.5 dilution series with a maximal concentration of 2.5 μM. When investigating the effect of NADH concentration upon inhibition of 17β-HSD10 by the inhibitors AcAc was kept in excess at 800 μM and NADH diluted down from 150 M. When investigating the effect of AcAc it was diluted from 1000 μM with an excess of 100 μM NADH.

Both inhibitors appear to have a similar mechanism of action. For NADH at increasing concentration of inhibitor, a decrease in Vmax is observed with no change in Km. This is characteristic of non-competitive (mixed) inhibition and is reflected by convergence of the data to the left of the y axis in a Lineweaver-Burke plot. In contrast, for AcAc, an increase in Kmₐₚₚ at increasing inhibitor concentrations is observed, which is characteristic of competitive inhibition. This was supported by a Lineweaver-Burke plot. In conclusion, these data suggest that both ESC1002033 and ESC1002082 bind non-competitively with NADH but competitively with AcAc.

### Cell Viability Testing and Cytotoxicity Assays

The effect upon cellular proliferation was tested for all compounds in a liver HepG2 cell line by using an Invitrogen™ CyQUANT™ Cell proliferation assay and a resazurin-resorufin metabolic assay. Two cytotoxicity assays, CyQuant assay and CellVi assay were used on HepG2 cells to provide a measure of cytotoxicity and identify cytotoxic and cytostatic compounds. Overall, there were no issues identified with most compounds having no measurable EC₅₀. Lactate dehydrogenase assays (LDH) and Alamar blue assays in HEK293 cells were also used to probe cytotoxicity and cell viability, however no real cytotoxicity was measured with all compounds showing <15% cytotoxicity (see Table 6).

### Enzyme Activity Cell Based Assay

A selection of the most potent compounds were profiled in HEK 293 cells (human embryonic kidney) utilising a fluorogenic probe -(-)CHANA (Muirhead et al. 2010

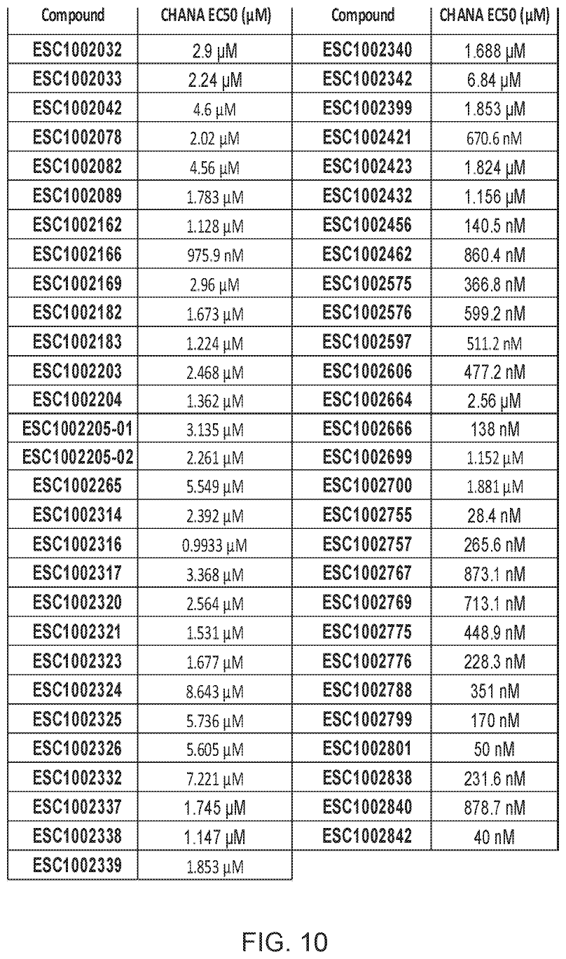

REF: (-)-CHANA, a Fluorogenic Probe for Detecting Amyloid Binding Alcohol Dehydrogenase HSD10 Activity in Living Cells Kirsty E. A. Muirhead, Mary Froemming, Xiaoguang Li, Kamil Musilek, Stuart J. Conway, Dalibor Sames, and Frank J. Gunn-Moore ACS Chemical Biology 2010 5 (12), 1105-1114DOI: 10.1021/cb100199m) to monitor the oxidative activity of 17β-HSD10 in living cells. Cellular profiling of the compounds indicated a good correlation between the biochemical and cellular assays with the results of most compounds falling within 10-fold of the results of the biochemical assay. This indicates that cellular drop off is not an issue. Furthermore, several of the compounds were found to be very potent inhibitors of cellular 17β-HSD10, having EC₅₀ of <100 nM. ESC1002755, the most potent, had an EC₅₀ of 28 nM (see FIG. 10).

### Crystallography and Molecular Modelling

Crystal structures of representatives from both analogue series were obtained. The X-ray data for all complexes was of high quality with well-defined electron density for the ligands (see Table 4a, b, c). Molecular replacement was carried out using Phaser and model building/refinement were carries out with CCP4/Coot/Phenix. Refinement statistics for the models are shown below. One representative of each series is discussed in more detail.

TABLE 4
X-ray crystallography data resolved for three
ligand-compound structures (a, b, c).

| ESC1002033 | |
|---|---|
| Space Group | P6₁ |
| Cell Dimensions | 133.25, 133.25, 133.89, 90, 90, 120 |
| No. Unique Reflections | 86875 |
| Resolution | 66.6-2.03 |
| Rwork/Rfree | 18.0/20.6 |
| Ramachandran (favoured/outlier) | 95.4%/0.3% |
| Bond Length/Angle rmsd | 0.011/1.656 |
| ESC1002421 | |
| Space Group | P6₁ |
| Cell Dimensions | 131.77, 131.77, 132.84, 90, 90, 120 |
| No. Unique Reflections | 68115 |
| Resolution | 43.3-2.18 |
| Rwork/Rfree | 17.3/20.4 |
| Ramachandran (favoured/outlier) | 95.7%/0.2% |
| Bond Length/Angle rmsd | 0.008/1.202 |
| ESC1002332 | |
| Space Group | P6₁ |
| Cell Dimensions | 131.33, 131.33, 132.60, 90, 90, 120 |
| No. Unique Reflections | 69438 |
| Resolution | 113.7-2.16 |
| Rwork/Rfree | 17.8/20.9 |
| Ramachandran (favoured/outlier) | 95.9%/0.3% |
| Bond Length/Angle rmsd | 0.007/1.047 |

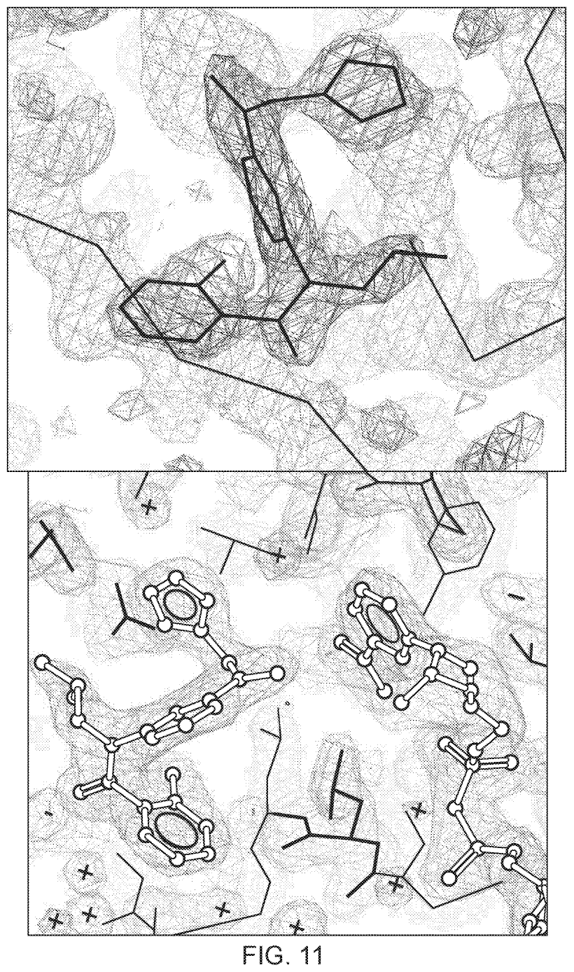

All ligands showed convincing mFo-DFc difference density and also for the nicotinamide cofactor. The models were built with coot and refined iteratively with phenix and with the CCP4 package to a final Rfactor of below 20% and with reasonable stereochemistry. The ligands bound in the same deep, predominantly hydrophobic pocket and shared some notable binding interactions. Example electron density is shown in FIG. 11 with initial electron density mFo-DFc in green on the left (ligand superimposed for reference) and the 2mFo-Dfc density for the fitted ligand and cofactor on the right. Ligand complexes were obtained either by cocrystallisation with ESC 1002033 or by cocrystallising then soaking out the first ligand and replacing it with another. There are four molecules in the asymmetric unit and in some cases, the soaking procedure did not replace the original cocrystallisation ligand in all molecules. The relevant ligand was built and refined in each case.

### ESC1002033

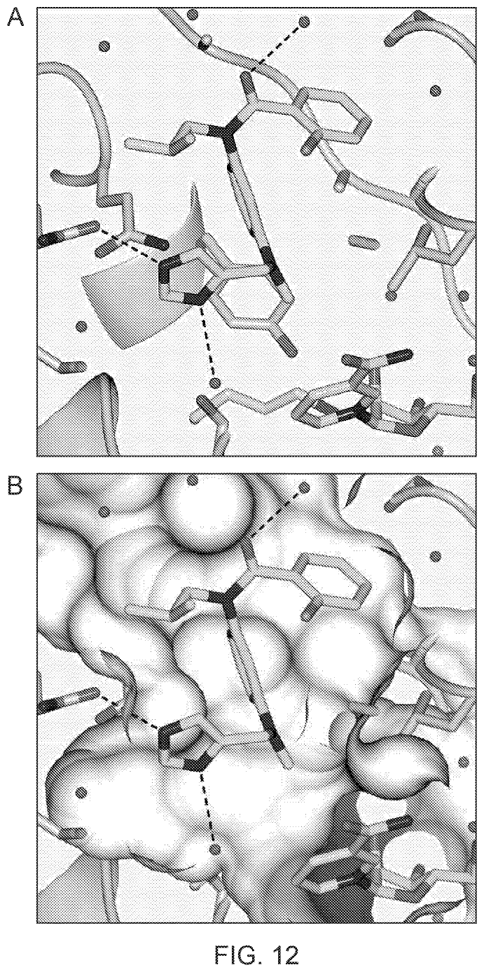

Binding site (left) and surface (right) representations of the ligand are shown in FIG. 12. The fluorophenyl ring is almost perpendicular to the plane of the amide bond. Breaking conjugation between these two systems is energetically unfavourable and is likely to be caused by steric effects from the central phenyl ring and the ortho fluorine. Placing bulkier substituents here to pre-organise the ligand in the bound conformation resulted in equipotent or marginally more potent compounds. The imidazole ring is at the centre of a hydrogen bond network. This part of the side is deeply buried and will have a low relative permittivity and the effects of electrostatic interactions will be increased as a consequence. Another feature of this pocket is a well-ordered water molecule at the base of a sub-pocket and accessible through a vector from the imidazole ring. The methyl isopropyl group occupies a quite constricted pocket with a small amount of elaboration possible. Some substitution here may be beneficial to crowd the space around the amide group and force the fluorophenyl group into the unfavoured conformation.

Interaction of the ligand with the cofactor was mediated through a methyl group. Investigation of the small molecule database showed that this was quite a common interaction and presumably relies on the delta-positive hydrogen atoms of the methyl interacting with the delocalised electrons of the nicotinamide ring.

### ESC1002332

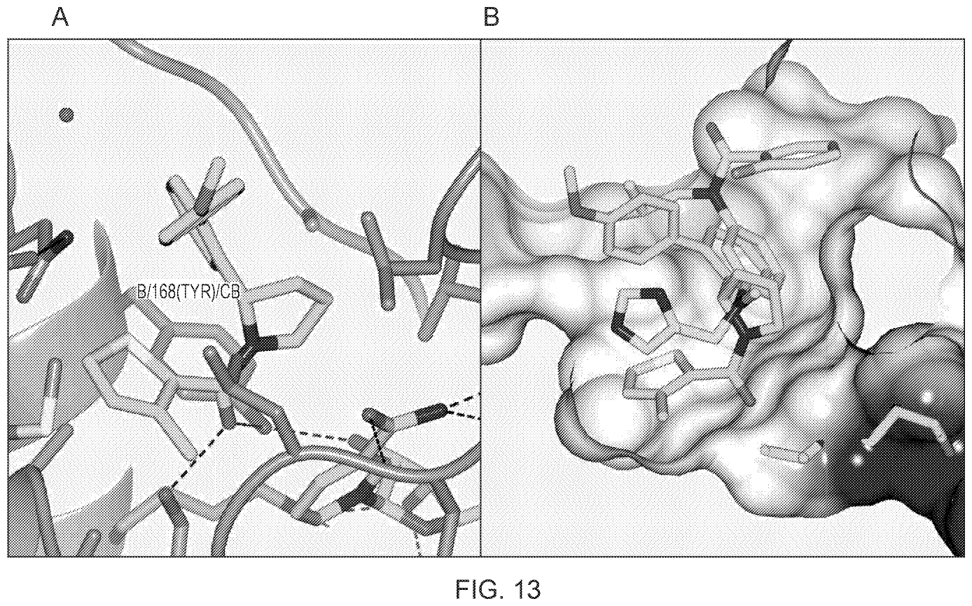

The ligand bound in the same site as ESC1002033 (FIG. 13a), sharing some interactions but others vary significantly (FIG. 13b). A key shared feature is the edge/face interaction of the central aryl ring of the ligand with tyrosine 168 at the base of the active site pocket. The thiophene ring occupies a somewhat more buried space compared with the imidazole ring of ESC1002033 and both molecules have aromatic groups extending towards the solvent. ESC1002332 also forms a direct hydrogen bond to tyrosine 168.

A notable difference in the two complexes is the course of the ligands as they emerge from the binding site towards solvent. Both chemotypes superimpose well at the core phenyl, but diverge as they extend towards solvent.

### ADME Profiling

Initial mouse hepatocyte stability and plasma protein binding experiments were undertaken on some of the most potent exemplars from the representative series and their results are summarised below in Table 5. The compounds were found to be either moderately or highly metabolised in mouse hepatocytes. The cluster exemplars appear more stable in comparison with the singleton exemplars.

Table 5: Initial mouse hepatocyte stability and plasma protein binding experiments were undertaken on some of the best exemplars from the representative series with AG18051 a previously published 17β-HSD10 inhibitor (C. R. Kissinger et al., J. Mol. Biol., 342 (2004), p. 943; Lim, Y.-A., Grimm, A., Giese, M. PLoS ONE (2011), 6, e28887.)
Ref: shown for comparison. Where k=absorption rate constant Cl=clearance, T_{1/2}=half-life, Heps=hepatocyte metabolism, PPB=plasma protein binding.

| | Mean | | Cl ml/ | | | Mouse |
|---|---|---|---|---|---|---|
| | ABAD | | minute/ | T_{1/2} | Heps | PPB |
| ESC Code | pIC50 | k | g liver | (minutes) | Category | (% free) |
| ESC1002755 | 7.73 | 0.22 | 105.8 | 3.14 | High | |
| ESC1002842 | 7.46 | 0.17 | 83.1 | 4 | High | 3.4 |
| ESC1002757 | 6.98 | 0.13 | 62.4 | 5.33 | High | |
| ESC1002456 | 6.86 | 0.018 | 8.6 | 38.48 | High | 8.9 |
| ESC1002799 | 6.96 | 0.046 | 21.9 | 15.18 | High | 9.9 |
| ESC1002769 | 6.77 | 0.15 | 71.5 | 4.65 | High | |
| ESC1002033 | 6.7 | 0.013 | 6.2 | 53.82 | Mod | |
| ESC1002575 | 6.57 | 0.012 | 5.8 | 57.15 | Mod | 7.6 |
| ESC1002204 | 6.4 | 0.11 | 54 | 6.16 | High | |
| ESC1002597 | 6.4 | 0.02 | 7.2 | 46.19 | Mod | |
| AG18051 | | 0.11 | 50.5 | 6.58 | High | |

TABLE 6
Compound structures and results of recombinant 17βHSD10 enzyme
assays

| | | | | Max. | | |
|---|---|---|---|---|---|---|
| | Compound | Max. | Min. | Effect | Hill | |
| Compound Structure | Reference | Fit | Fit | (%) | Slope | pEC₅₀ |
| 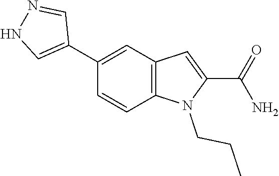 [SMILES: CCCn1c(C(N)=O)cc2cc(-c3cn[nH]c3)ccc21] | ESC1002019 | 93 | 2 | 85 | 1.3 | 5.4 |
| 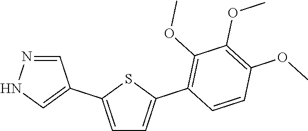 [SMILES: COc1ccc(-c2ccc(-c3cn[nH]c3)s2)c(OC)c1OC] | ESC1002024 | 101 | 2 | 89 | 1.2 | 5.4 |
| 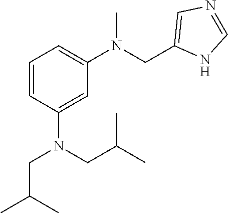 [SMILES: CC(C)CN(CC(C)C)c1cccc(N(C)Cc2cnc[nH]2)c1] | ESC1002025 | | | 15 | | <4.7 |
| 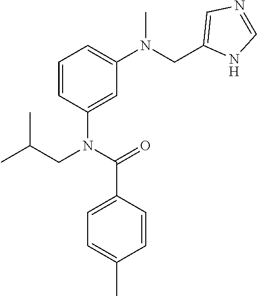 [SMILES: Cc1ccc(C(=O)N(CC(C)C)c2cccc(N(C)Cc3cnc[nH]3)c2)cc1] | ESC1002032 | 99 | 0 | 99 | 0.9 | 6.6 |
| 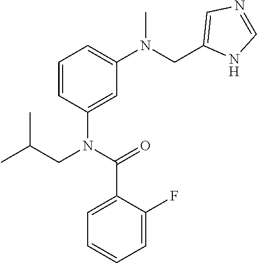 [SMILES: CC(C)CN(C(=O)c1ccccc1F)c1cccc(N(C)Cc2cnc[nH]2)c1] | ESC1002033 | 99 | −3 | 99 | 1 | 6.7 |
| 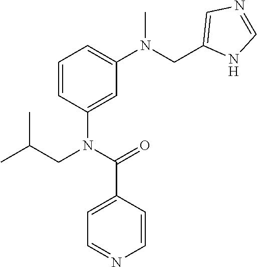 [SMILES: CC(C)CN(C(=O)c1ccncc1)c1cccc(N(C)Cc2cnc[nH]2)c1] | ESC1002034 | 106 | −3 | 92 | 0.8 | 5.8 |
| 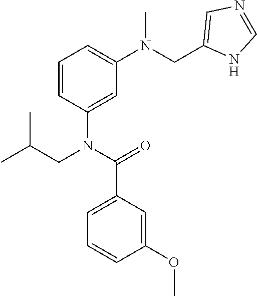 [SMILES: COc1cccc(C(=O)N(CC(C)C)c2cccc(N(C)Cc3cnc[nH]3)c2)c1] | ESC1002038 | 105 | −2 | 92 | 0.8 | 5.7 |
| 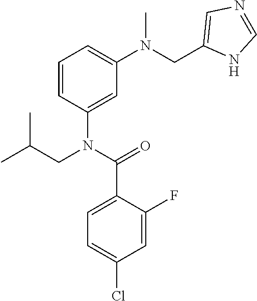 [SMILES: CC(C)CN(C(=O)c1ccc(Cl)cc1F)c1cccc(N(C)Cc2cnc[nH]2)c1] | ESC1002042 | 101 | −6 | 98 | 0.8 | 6.5 |
| 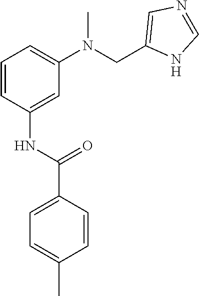 [SMILES: Cc1ccc(C(=O)Nc2cccc(N(C)Cc3cnc[nH]3)c2)cc1] | ESC1002054 | | | 3 | | <4.7 |
| 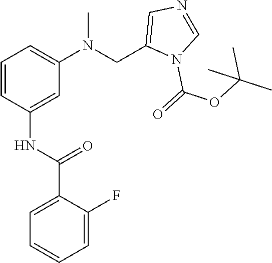 [SMILES: CN(Cc1cncn1C(=O)OC(C)(C)C)c1cccc(NC(=O)c2ccccc2F)c1] | ESC1002055 | | | 1 | | <4.7 |
| 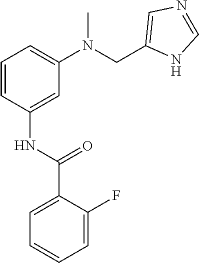 [SMILES: CN(Cc1cnc[nH]1)c1cccc(NC(=O)c2ccccc2F)c1] | ESC1002056 | | | 4 | | <4.7 |
| 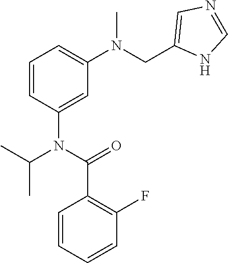 [SMILES: CC(C)N(C(=O)c1ccccc1F)c1cccc(N(C)Cc2cnc[nH]2)c1] | ESC1002078 | 101 | −1 | 97 | 1 | 6.2 |
| 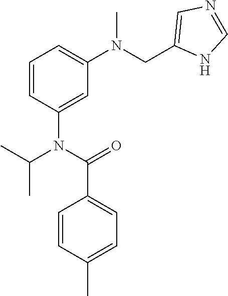 [SMILES: Cc1ccc(C(=O)N(c2cccc(N(C)Cc3cnc[nH]3)c2)C(C)C)cc1] | ESC1002080 | 102 | 0 | 96 | 0.9 | 6 |
| 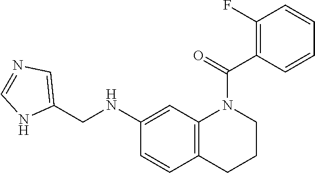 [SMILES: O=C(c1ccccc1F)N1CCCc2ccc(NCc3cnc[nH]3)cc21] | ESC1002081 | | | 0 | | <4.7 |
| 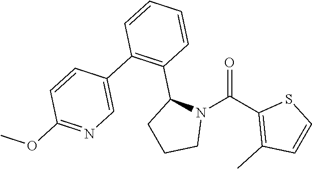 [SMILES: COc1ccc(-c2ccccc2[C@@H]2CCCN2C(=O)c2sccc2C)cn1] | ESC1002082 | 98 | 0 | 96 | 1.1 | 6.1 |
| 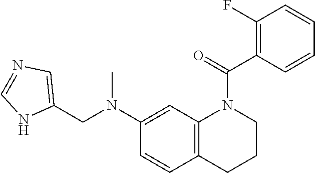 [SMILES: CN(Cc1cnc[nH]1)c1ccc2c(c1)N(C(=O)c1ccccc1F)CCC2] | ESC1002083 | | | 2 | | <4.7 |
| 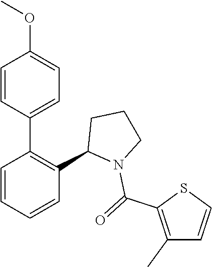 [SMILES: COc1ccc(-c2ccccc2[C@H]2CCCN2C(=O)c2sccc2C)cc1] | ESC1002089 | 98 | 0 | 100 | 1.1 | 6.48 |
| 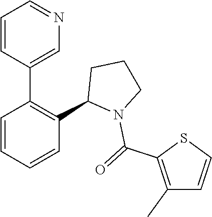 [SMILES: Cc1ccsc1C(=O)N1CCC[C@@H]1c1ccccc1-c1cccnc1] | ESC1002090 | 97 | 0 | 94 | 1.1 | 5.94 |
| 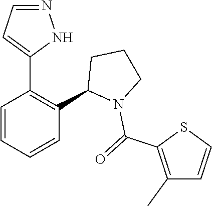 [SMILES: Cc1ccsc1C(=O)N1CCC[C@@H]1c1ccccc1-c1ccn[nH]1] | ESC1002091 | 93 | 0 0 | 84 | 1.2 | 5.5 |
| 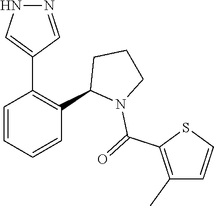 [SMILES: Cc1ccsc1C(=O)N1CCC[C@@H]1c1ccccc1-c1cn[nH]c1] | ESC1002092 | 98 | 0 | 93 | 1 | 5.91 |
| 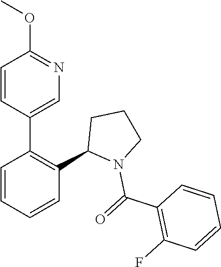 [SMILES: COc1ccc(-c2ccccc2[C@H]2CCCN2C(=O)c2ccccc2F)cn1] | ESC1002119 | 57 | 0 | 64 | 1.3 | 5.38 |
| 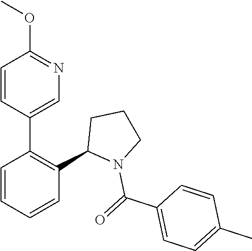 [SMILES: COc1ccc(-c2ccccc2[C@H]2CCCN2C(=O)c2ccc(C)cc2)cn1] | ESC1002120 | | | 13 | | <4.7 |
| 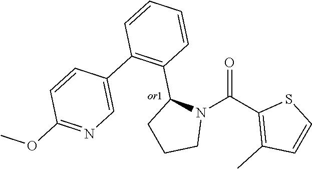 [SMILES: COc1ccc(-c2ccccc2[C@@H]2CCCN2C(=O)c2sccc2C)cn1] | ESC1002161 | | | 7 | | <4.7 |
| 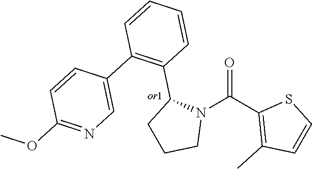 [SMILES: COc1ccc(-c2ccccc2[C@H]2CCCN2C(=O)c2sccc2C)cn1] | ESC1002162 | 99 | 0 | 98 | 1 | 6.31 |
| 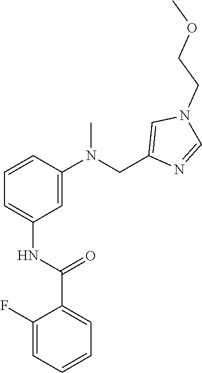 [SMILES: COCCn1cnc(CN(C)c2cccc(NC(=O)c3ccccc3F)c2)c1] | ESC1002163 | | | 0 | | <4.7 |
| 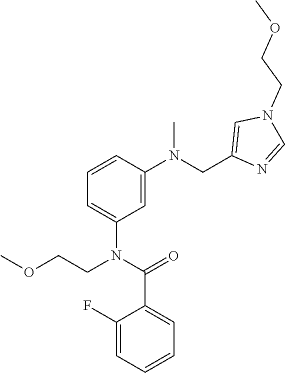 [SMILES: COCCN(C(=O)c1ccccc1F)c1cccc(N(C)Cc2cn(CCOC)cn2)c1] | ESC1002164 | | | 2 | | <4.7 |
| 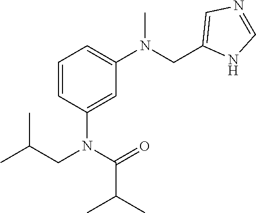 [SMILES: CC(C)CN(C(=O)C(C)C)c1cccc(N(C)Cc2cnc[nH]2)c1] | ESC1002165 | 100 | 0 | 94 | 1 | 5.78 |
| 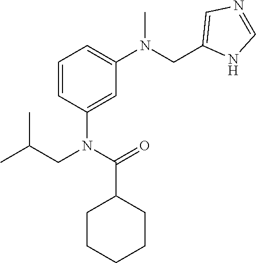 [SMILES: CC(C)CN(C(=O)C1CCCCC1)c1cccc(N(C)Cc2cnc[nH]2)c1] | ESC1002166 | 104 | 0 | 99 | 0.93 | 6.02 |
| 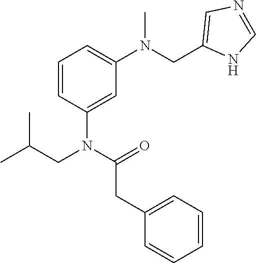 [SMILES: CC(C)CN(C(=O)Cc1ccccc1)c1cccc(N(C)Cc2cnc[nH]2)c1] | ESC1002167 | 100 | 0 | 62 | 1 | 4.9 |
| 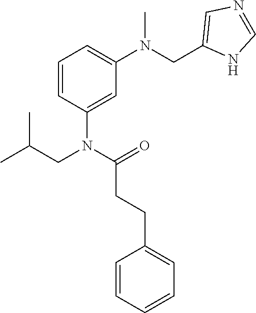 [SMILES: CC(C)CN(C(=O)CCc1ccccc1)c1cccc(N(C)Cc2cnc[nH]2)c1] | ESC1002168 | 100 | 0 | 72 | 1.1 | 5.05 |
| 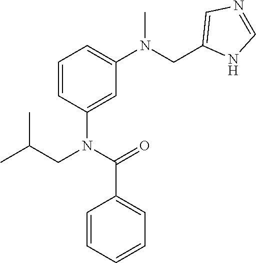 [SMILES: CC(C)CN(C(=O)c1ccccc1)c1cccc(N(C)Cc2cnc[nH]2)c1] | ESC1002169 | 98 | 0 | 97 | 1.1 | 6.19 |
| 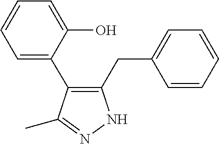 [SMILES: Cc1n[nH]c(Cc2ccccc2)c1-c1ccccc1O] | ESC1002170 | 98 | 0 | 99 | 1.1 | 6.3 |
| 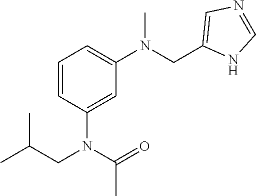 [SMILES: CC(=O)N(CC(C)C)c1cccc(N(C)Cc2cnc[nH]2)c1] | ESC1002171 | | | 34 | | <4.7 |
| 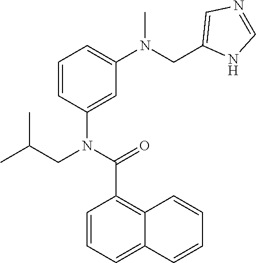 [SMILES: CC(C)CN(C(=O)c1cccc2ccccc12)c1cccc(N(C)Cc2cnc[nH]2)c1] | ESC1002172 | 96 | 0 | 87 | 1.1 | 5.55 |
| 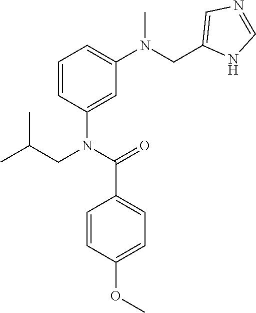 [SMILES: COc1ccc(C(=O)N(CC(C)C)c2cccc(N(C)Cc3cnc[nH]3)c2)cc1] | ESC1002182 | 103 | 0 | 99 | 0.8 | 6.48 |
| 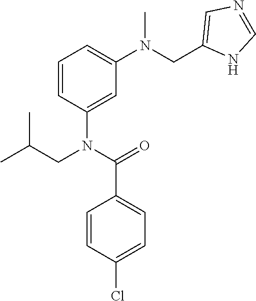 [SMILES: CC(C)CN(C(=O)c1ccc(Cl)cc1)c1cccc(N(C)Cc2cnc[nH]2)c1] | ESC1002183 | 97 | 0 | 95 | 1.2 | 6.11 |
| 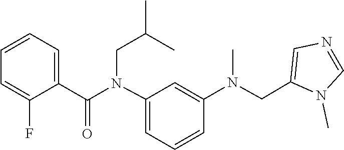 [SMILES: CC(C)CN(C(=O)c1ccccc1F)c1cccc(N(C)Cc2cncn2C)c1] | ESC1002199 | | | 13 | | <4.7 |
| 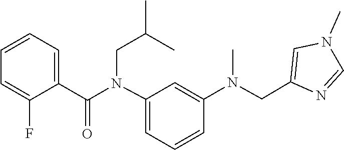 [SMILES: CC(C)CN(C(=O)c1ccccc1F)c1cccc(N(C)Cc2cn(C)cn2)c1] | ESC1002200 | 96 | 0 | 67 | 1 | 5.04 |
| 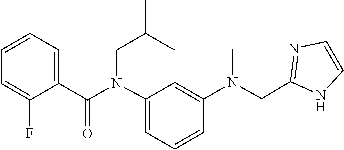 [SMILES: CC(C)CN(C(=O)c1ccccc1F)c1cccc(N(C)Cc2ncc[nH]2)c1] | ESC1002201 | | | 41 | | <4.7 |
| 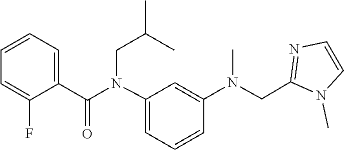 [SMILES: CC(C)CN(C(=O)c1ccccc1F)c1cccc(N(C)Cc2nccn2C)c1] | ESC1002202 | 79 | 0 | 57 | 1.3 | 5 |
| 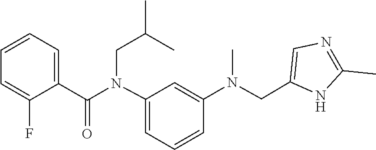 [SMILES: Cc1ncc(CN(C)c2cccc(N(CC(C)C)C(=O)c3ccccc3F)c2)[nH]1] | ESC1002203 | 98 | 0 | 97 | 1.1 | 6.28 |
| 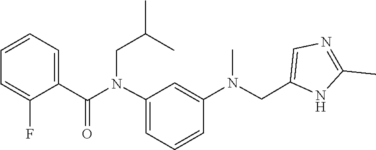 [SMILES: Cc1ncc(CN(C)c2cccc(N(CC(C)C)C(=O)c3ccccc3F)c2)[nH]1] | ESC1002204 | 98 | 0 | 97 | 1.2 | 6.4 |
| 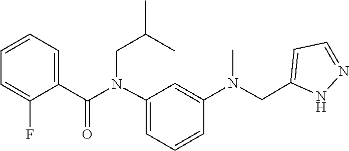 [SMILES: CC(C)CN(C(=O)c1ccccc1F)c1cccc(N(C)Cc2ccn[nH]2)c1] | ESC1002205 | 97 | 0 | 96 | 1.1 | 6.05 |
| 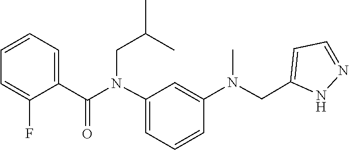 [SMILES: CC(C)CN(C(=O)c1ccccc1F)c1cccc(N(C)Cc2ccn[nH]2)c1] | ESC1002206 | 107 | 0 | 79 | 0.93 | 5.18 |
| 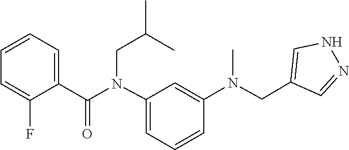 [SMILES: CC(C)CN(C(=O)c1ccccc1F)c1cccc(N(C)Cc2cn[nH]c2)c1] | ESC1002205 | 98 | 0 | 96 | 1.2 | 6.04 |
| 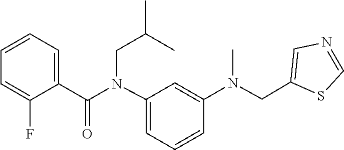 [SMILES: CC(C)CN(C(=O)c1ccccc1F)c1cccc(N(C)Cc2cncs2)c1] | ESC1002208 | | | 50 | | <4.7 |
| 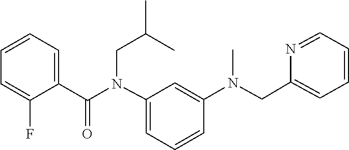 [SMILES: CC(C)CN(C(=O)c1ccccc1F)c1cccc(N(C)Cc2ccccn2)c1] | ESC1002209 | 104 | 0 | 80 | 0.98 | 5.24 |
| 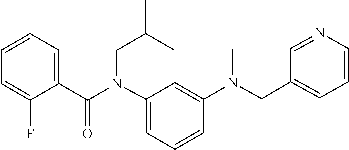 [SMILES: CC(C)CN(C(=O)c1ccccc1F)c1cccc(N(C)Cc2cccnc2)c1] | ESC1002210 | 82 | 0 | 72 | 1.4 | 5.21 |
| 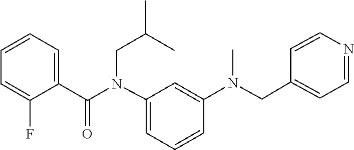 [SMILES: CC(C)CN(C(=O)c1ccccc1F)c1cccc(N(C)Cc2ccncc2)c1] | ESC1002211 | | | 40 | | <4.7 |
| 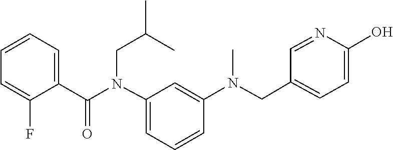 [SMILES: CC(C)CN(C(=O)c1ccccc1F)c1cccc(N(C)Cc2ccc(O)nc2)c1] | ESC1002212 | | | 16 | | <4.7 |
| 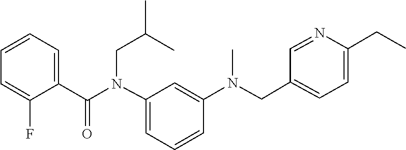 [SMILES: CCc1ccc(CN(C)c2cccc(N(CC(C)C)C(=O)c3ccccc3F)c2)cn1] | ESC1002213 | | | 15 | | <4.7 |
| 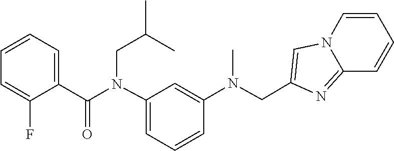 [SMILES: CC(C)CN(C(=O)c1ccccc1F)c1cccc(N(C)Cc2cn3ccccc3n2)c1] | ESC1002214 | | | 22 | | <4.7 |
| 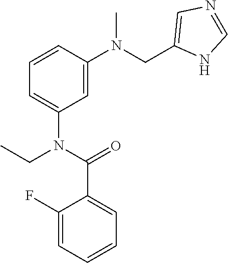 [SMILES: CCN(C(=O)c1ccccc1F)c1cccc(N(C)Cc2cnc[nH]2)c1] | ESC1002234 | 101 | −3 | 94 | 0.96 | 5.83 |
| 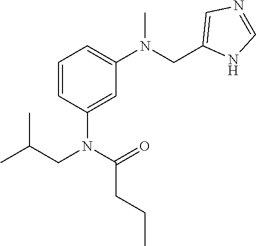 [SMILES: CCCC(=O)N(CC(C)C)c1cccc(N(C)Cc2cnc[nH]2)c1] | ESC1002235 | 93 | −3 | 85 | 1.1 | 5.53 |
| 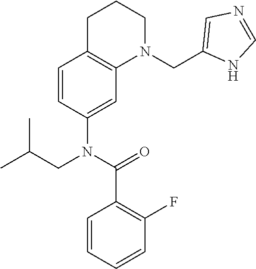 [SMILES: CC(C)CN(C(=O)c1ccccc1F)c1ccc2c(c1)N(Cc1cnc[nH]1)CCC2] | ESC1002236 | | | 30 | | <4.7 |
| 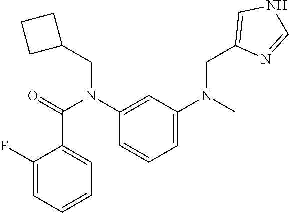 [SMILES: CN(Cc1c[nH]cn1)c1cccc(N(CC2CCC2)C(=O)c2ccccc2F)c1] | ESC1002265 | 99 | 0 | 96 | 1.1 | 6.02 |
| 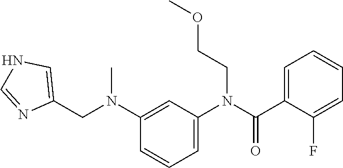 [SMILES: COCCN(C(=O)c1ccccc1F)c1cccc(N(C)Cc2c[nH]cn2)c1] | ESC1002266 | 100 | −4 | 74 | 0.98 | 5.19 |
| 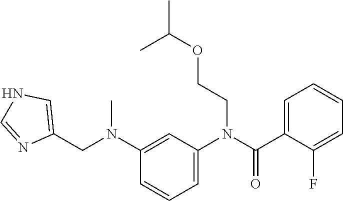 [SMILES: CC(C)OCCN(C(=O)c1ccccc1F)c1cccc(N(C)Cc2c[nH]cn2)c1] | ESC1002267 | 103 | −4 | 76 | 0.9 | 5.22 |
| 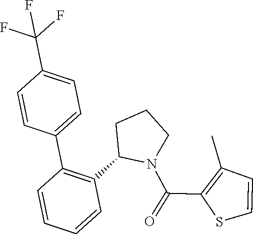 [SMILES: Cc1ccsc1C(=O)N1CCC[C@H]1c1ccccc1-c1ccc(C(F)(F)F)cc1] | ESC1002314 | 97 | 0 | 99 | 1.2 | 6.24 |
| 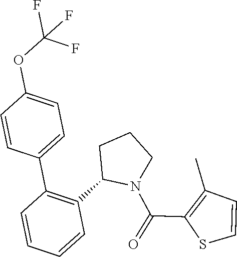 [SMILES: Cc1ccsc1C(=O)N1CCC[C@H]1c1ccccc1-c1ccc(OC(F)(F)F)cc1] | ESC1002315 | 97 | 0 | 95 | 1.1 | 5.94 |
| 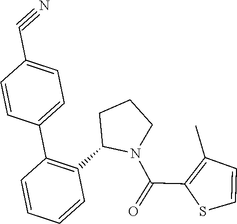 [SMILES: Cc1ccsc1C(=O)N1CCC[C@H]1c1ccccc1-c1ccc(C#N)cc1] | ESC1002316 | 100 | 0 | 101 | 0.93 | 6.56 |
| 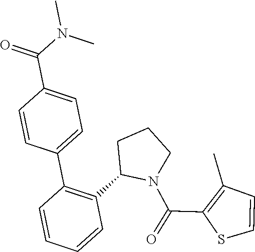 [SMILES: Cc1ccsc1C(=O)N1CCC[C@H]1c1ccccc1-c1ccc(C(=O)N(C)C)cc1] | ESC1002317 | 99 | 0 | 96 | 1 | 6.04 |
| 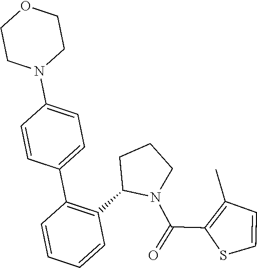 [SMILES: Cc1ccsc1C(=O)N1CCC[C@H]1c1ccccc1-c1ccc(N2CCOCC2)cc1] | ESC1002318 | 98 | −1 | 91 | 1.1 | 5.72 |
| 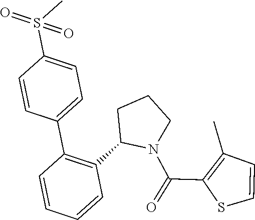 [SMILES: Cc1ccsc1C(=O)N1CCC[C@H]1c1ccccc1-c1ccc(S(C)(=O)=O)cc1] | ESC1002319 | 101 | −2 | 79 | 1 | 5.23 |
| 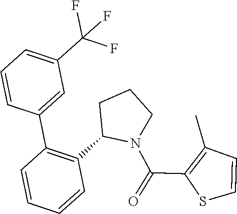 [SMILES: Cc1ccsc1C(=O)N1CCC[C@H]1c1ccccc1-c1cccc(C(F)(F)F)c1] | ESC1002320 | 101 | −1 | 99 | 0.91 | 6.22 |
| 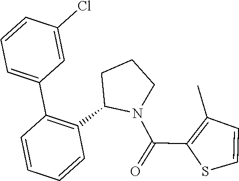 [SMILES: Cc1ccsc1C(=O)N1CCC[C@H]1c1ccccc1-c1cccc(Cl)c1] | ESC1002321 | 102 | 4 | 105 | 0.92 | 6.58 |
| 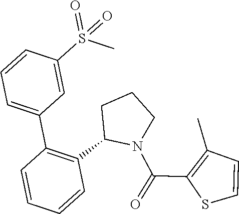 [SMILES: Cc1ccsc1C(=O)N1CCC[C@H]1c1ccccc1-c1cccc(S(C)(=O)=O)c1] | ESC1002322 | | | 41 | | <4.7 |
| 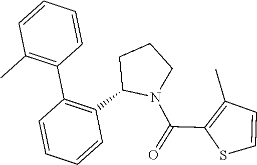 [SMILES: Cc1ccccc1-c1ccccc1[C@@H]1CCCN1C(=O)c1sccc1C] | ESC1002323 | 101 | 7 | 106 | 1.4 | 6.33 |
| 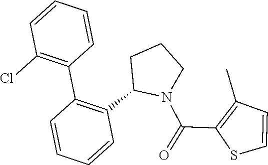 [SMILES: Cc1ccsc1C(=O)N1CCC[C@H]1c1ccccc1-c1ccccc1Cl] | ESC1002324 | 102 | 0 | 99 | 1 | 6.53 |
| 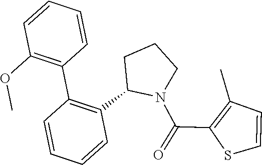 [SMILES: COc1ccccc1-c1ccccc1[C@@H]1CCCN1C(=O)c1sccc1C] | ESC1002325 | 100 | −11 | 98 | 0.87 | 6.51 |
| 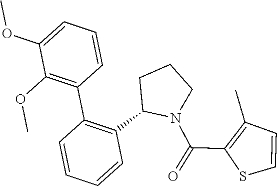 [SMILES: COc1cccc(-c2ccccc2[C@@H]2CCCN2C(=O)c2sccc2C)c1OC] | ESC1002326 | 99 | 4 | 97 | 1.1 | 6.15 |
| 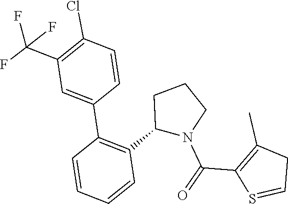 | ESC1002327 | 100 | −7 | 89 | 0.84 | 5.84 |
| 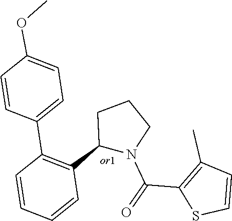 [SMILES: COc1ccc(-c2ccccc2[C@H]2CCCN2C(=O)c2sccc2C)cc1] | ESC1002089 | | | 18 | | <4.7 |
| 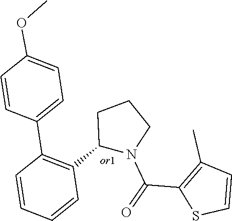 [SMILES: COc1ccc(-c2ccccc2[C@@H]2CCCN2C(=O)c2sccc2C)cc1] | ESC1002332 | 100 | 0 | 99 | 1 | 6.78 |
| 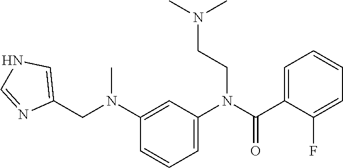 [SMILES: CN(C)CCN(C(=O)c1ccccc1F)c1cccc(N(C)Cc2c[nH]cn2)c1] | ESC1002334 | | | 23 | | <4.7 |
| 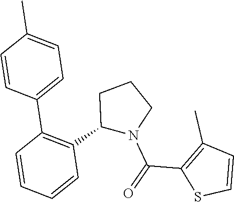 [SMILES: Cc1ccc(-c2ccccc2[C@@H]2CCCN2C(=O)c2sccc2C)cc1] | ESC1002337 | 99 | −7 | 102 | 1.3 | 6.68 |
| 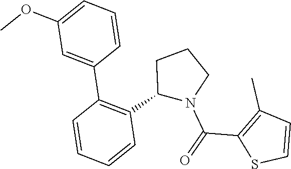 [SMILES: COc1cccc(-c2ccccc2[C@@H]2CCCN2C(=O)c2sccc2C)c1] | ESC1002338 | 100 | 0 | 101 | 1.1 | 6.72 |
| 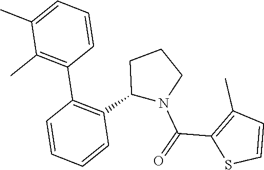 [SMILES: Cc1ccsc1C(=O)N1CCC[C@H]1c1ccccc1-c1cccc(C)c1C] | ESC1002339 | 97 | 7 | 99 | 1.4 | 6.28 |
| 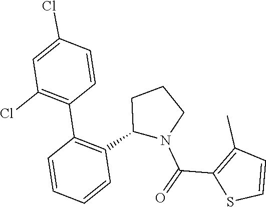 [SMILES: Cc1ccsc1C(=O)N1CCC[C@H]1c1ccccc1-c1ccc(Cl)cc1Cl] | ESC1002340 | 96 | −7 | 105 | 1.6 | 6.2 |
| 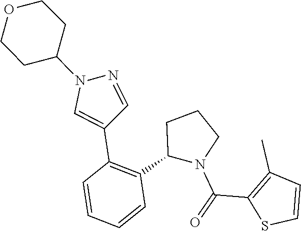 [SMILES: Cc1ccsc1C(=O)N1CCC[C@H]1c1ccccc1-c1cnn(C2CCOCC2)c1] | ESC1002341 | 102 | −6 | 91 | 0.87 | 5.79 |
| 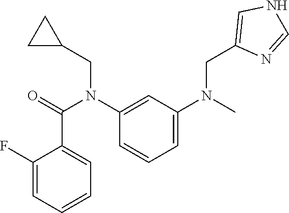 [SMILES: CN(Cc1c[nH]cn1)c1cccc(N(CC2CC2)C(=O)c2ccccc2F)c1] | ESC1002342 | 95 | −1 | 99 | 1.6 | 6.15 |
| 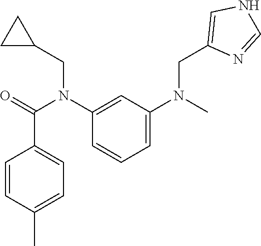 [SMILES: Cc1ccc(C(=O)N(CC2CC2)c2cccc(N(C)Cc3c[nH]cn3)c2)cc1] | ESC1002399 | 99 | 2 | 95 | 1.1 | 6 |
| 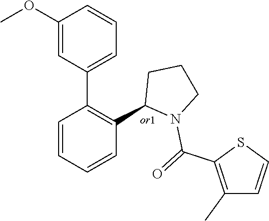 [SMILES: COc1cccc(-c2ccccc2[C@H]2CCCN2C(=O)c2sccc2C)c1] | ESC1002420 | | | 28 | | <4.7 |
| 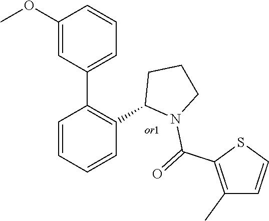 [SMILES: COc1cccc(-c2ccccc2[C@@H]2CCCN2C(=O)c2sccc2C)c1] | ESC1002421 | 95 | 11 | 97 | 1.6 | 6.73 |
| 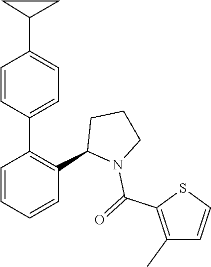 [SMILES: Cc1ccsc1C(=O)N1CCC[C@@H]1c1ccccc1-c1ccc(C2CC2)cc1] | ESC1002422 | 90 | 1 | 93 | 1.6 | 6.25 |
| 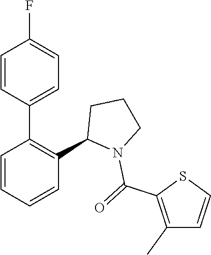 [SMILES: Cc1ccsc1C(=O)N1CCC[C@@H]1c1ccccc1-c1ccc(F)cc1] | ESC1002423 | 93 | 9 | 96 | 2.3 | 6.42 |
| 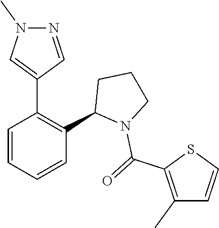 [SMILES: Cc1ccsc1C(=O)N1CCC[C@@H]1c1ccccc1-c1cnn(C)c1] | ESC1002424 | 96 | 2 | 96 | 1.3 | 6.03 |
| 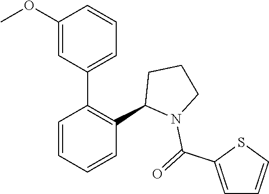 [SMILES: COc1cccc(-c2ccccc2[C@H]2CCCN2C(=O)c2cccs2)c1] | ESC1002425 | 95 | −8 | 96 | 1.1 | 6.32 |
| 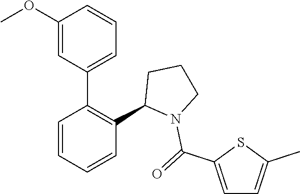 | ESC1002426 | | | 22 | | <4.7 |
| 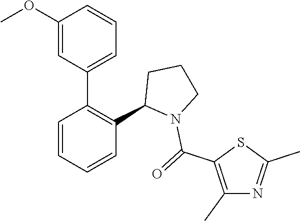 | ESC1002427 | | | 4 | | <4.7 |
| 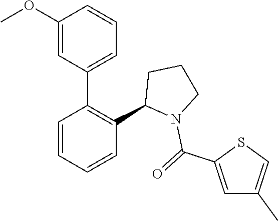 | ESC1002428 | | | 16 | | <4.7 |
| 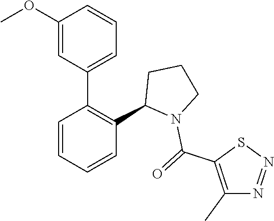 [SMILES: COc1cccc(-c2ccccc2[C@H]2CCCN2C(=O)c2snnc2C)c1] | ESC1002429 | | | 6 | | <4.7 |
| 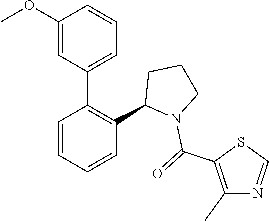 [SMILES: COc1cccc(-c2ccccc2[C@H]2CCCN2C(=O)c2scnc2C)c1] | ESC1002430 | 80 | −1 | 65 | 1.2 | 5.22 |
| 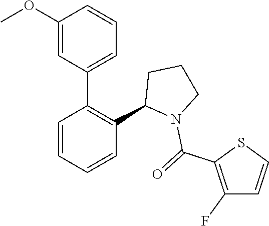 [SMILES: COc1cccc(-c2ccccc2[C@H]2CCCN2C(=O)c2sccc2F)c1] | ESC1002431 | 95 | 1 | 96 | 1.5 | 6.01 |
| 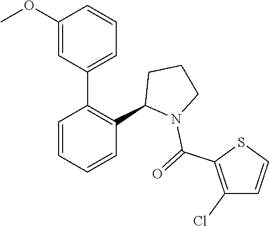 [SMILES: COc1cccc(-c2ccccc2[C@H]2CCCN2C(=O)c2sccc2Cl)c1] | ESC1002432 | 98 | −17 | 96 | 0.78 | 7.03 |
| 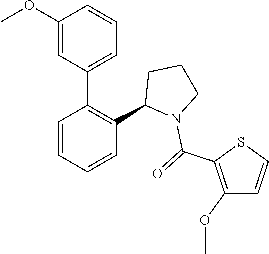 [SMILES: COc1cccc(-c2ccccc2[C@H]2CCCN2C(=O)c2sccc2OC)c1] | ESC1002433 | | | 17 | | <4.7 |
| 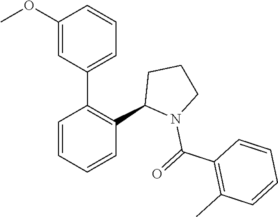 [SMILES: COc1cccc(-c2ccccc2[C@H]2CCCN2C(=O)c2ccccc2C)c1] | ESC1002434 | | | 20 | | <4.7 |
| 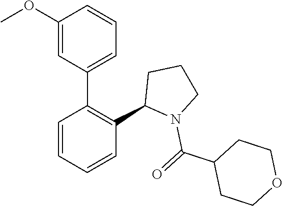 [SMILES: COc1cccc(-c2ccccc2[C@H]2CCCN2C(=O)C2CCOCC2)c1] | ESC1002435 | | | 12 | | <4.7 |
| 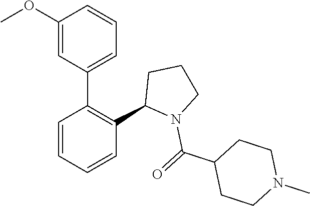 | ESC1002436 | | | 2 | | <4.7 |
| 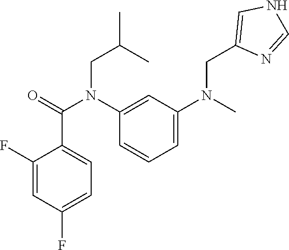 [SMILES: CC(C)CN(C(=O)c1ccc(F)cc1F)c1cccc(N(C)Cc2c[nH]cn2)c1] | ESC1002454 | 97 | −8 | 96 | 1.1 | 6.34 |
| 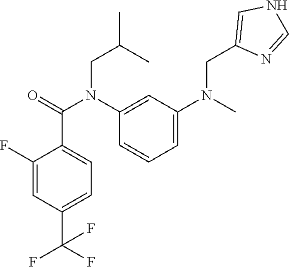 [SMILES: CC(C)CN(C(=O)c1ccc(C(F)(F)F)cc1F)c1cccc(N(C)Cc2c[nH]cn2)c1] | ESC1002455 | 97 | −7 | 92 | 0.99 | 5.9 |
| 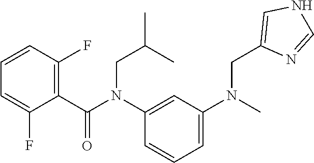 [SMILES: CC(C)CN(C(=O)c1c(F)cccc1F)c1cccc(N(C)Cc2c[nH]cn2)c1] | ESC1002456 | 96 | 17 | 97 | 1.2 | 6.86 |
| 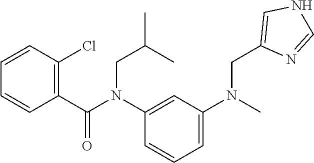 [SMILES: CC(C)CN(C(=O)c1ccccc1Cl)c1cccc(N(C)Cc2c[nH]cn2)c1] | ESC1002457 | 94 | −4 | 95 | 1.4 | 6.35 |
| 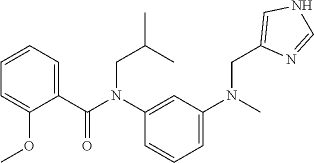 [SMILES: COc1ccccc1C(=O)N(CC(C)C)c1cccc(N(C)Cc2c[nH]cn2)c1] | ESC1002458 | 97 | −7 | 96 | 1 | 6.26 |
| 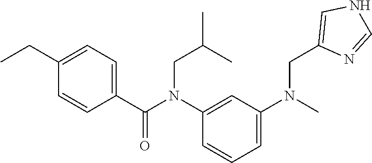 [SMILES: CCc1ccc(C(=O)N(CC(C)C)c2cccc(N(C)Cc3c[nH]cn3)c2)cc1] | ESC1002459 | 94 | −4 | 93 | 1.2 | 6.08 |
| 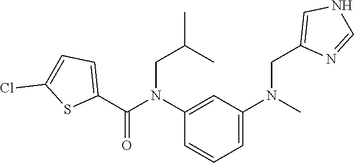 [SMILES: CC(C)CN(C(=O)c1ccc(Cl)s1)c1cccc(N(C)Cc2c[nH]cn2)c1] | ESC1002460 | 95 | −5 | 90 | 1 | 5.92 |
| 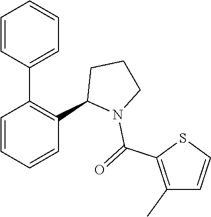 [SMILES: Cc1ccsc1C(=O)N1CCC[C@@H]1c1ccccc1-c1ccccc1] | ESC1002462 | 98 | 6 | 99 | 1.4 | 6.63 |
| 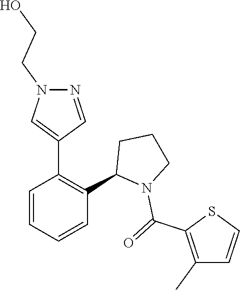 [SMILES: Cc1ccsc1C(=O)N1CCC[C@@H]1c1ccccc1-c1cnn(CCO)c1] | ESC1002463 | 110 | −6 | 87 | 0.79 | 5.46 |
| 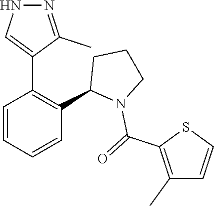 [SMILES: Cc1ccsc1C(=O)N1CCC[C@@H]1c1ccccc1-c1c[nH]nc1C] | ESC1002464 | 91 | −5 | 84 | 1.1 | 5.69 |
| 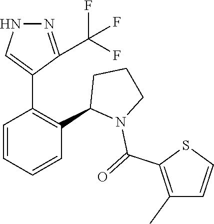 [SMILES: Cc1ccsc1C(=O)N1CCC[C@@H]1c1ccccc1-c1c[nH]nc1C(F)(F)F] | ESC1002465 | 90 | −2 | 87 | 1.2 | 5.82 |
| 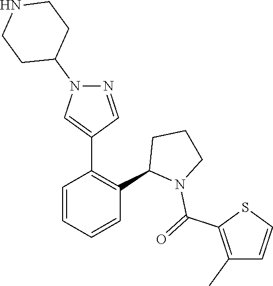 [SMILES: Cc1ccsc1C(=O)N1CCC[C@@H]1c1ccccc1-c1cnn(C2CCNCC2)c1] | ESC1002466 | 95 | −1 | 52 | 1 | 4.74 |
| 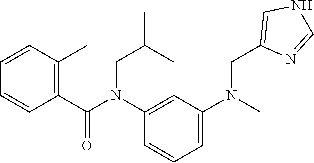 [SMILES: Cc1ccccc1C(=O)N(CC(C)C)c1cccc(N(C)Cc2c[nH]cn2)c1] | ESC1002491 | 102 | 2 | 96 | 1 | 5.93 |
| 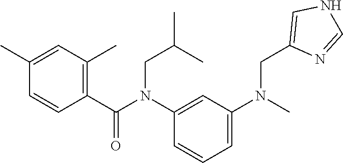 [SMILES: Cc1ccc(C(=O)N(CC(C)C)c2cccc(N(C)Cc3c[nH]cn3)c2)c(C)c1] | ESC1002492 | 104 | −10 | 93 | 0.73 | 6.06 |
| 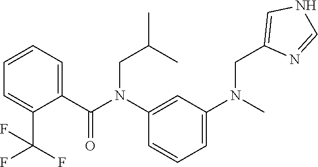 [SMILES: CC(C)CN(C(=O)c1ccccc1C(F)(F)F)c1cccc(N(C)Cc2c[nH]cn2)c1] | ESC1002493 | 104 | −8 | 94 | 0.75 | 6.01 |
| 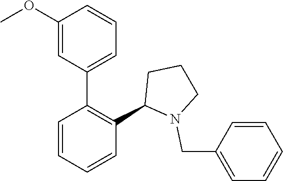 [SMILES: COc1cccc(-c2ccccc2[C@H]2CCCN2Cc2ccccc2)c1] | ESC1002504 | | | −6 | | <4.7 |
| 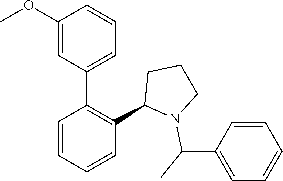 [SMILES: COc1cccc(-c2ccccc2[C@H]2CCCN2C(C)c2ccccc2)c1] | ESC1002505 | | | 3 | | <4.7 |
| 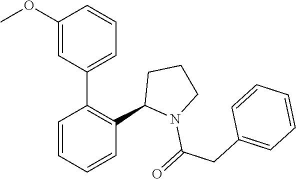 [SMILES: COc1cccc(-c2ccccc2[C@H]2CCCN2C(=O)Cc2ccccc2)c1] | ESC1002506 | | | 8 | | <4.7 |
| 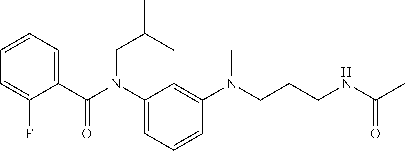 [SMILES: CC(=O)NCCCN(C)c1cccc(N(CC(C)C)C(=O)c2ccccc2F)c1] | ESC1002507 | | | 4 | | <4.7 |
| 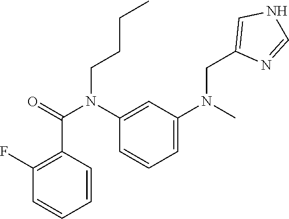 [SMILES: CCCCN(C(=O)c1ccccc1F)c1cccc(N(C)Cc2c[nH]cn2)c1] | ESC1002508 | 96 | 0 | 92 | 1.2 | 5.81 |
| 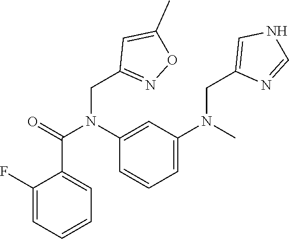 [SMILES: Cc1cc(CN(C(=O)c2ccccc2F)c2cccc(N(C)Cc3c[nH]cn3)c2)no1] | ESC1002516 | | | 46 | | <4.7 |
| 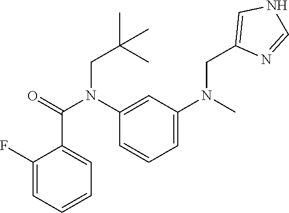 [SMILES: CN(Cc1c[nH]cn1)c1cccc(N(CC(C)(C)C)C(=O)c2ccccc2F)c1] | ESC1002517 | 106 | −1 | 92 | 0.87 | 5.7 |
| 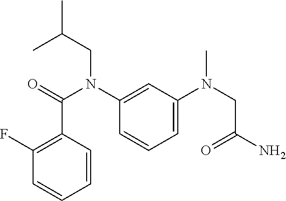 [SMILES: CC(C)CN(C(=O)c1ccccc1F)c1cccc(N(C)CC(N)=O)c1] | ESC1002570 | | | 48 | | <4.7 |
| 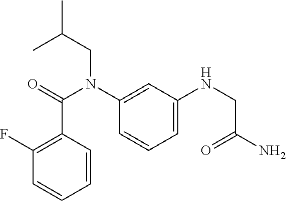 [SMILES: CC(C)CN(C(=O)c1ccccc1F)c1cccc(NCC(N)=O)c1] | ESC1002571 | | | 13 | | <4.7 |
| 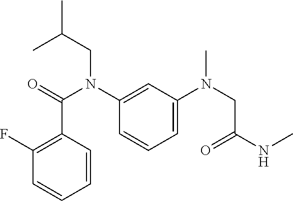 [SMILES: CNC(=O)CN(C)c1cccc(N(CC(C)C)C(=O)c2ccccc2F)c1] | ESC1002572 | 100 | 2 | 65 | 0.98 | 4.95 |
| 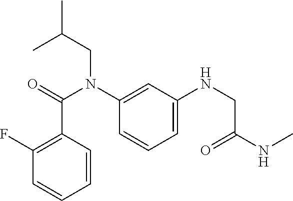 [SMILES: CNC(=O)CNc1cccc(N(CC(C)C)C(=O)c2ccccc2F)c1] | ESC1002573 | | | 4 | | <4.7 |
| 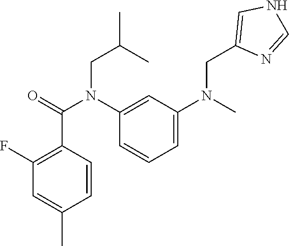 [SMILES: Cc1ccc(C(=O)N(CC(C)C)c2cccc(N(C)Cc3c[nH]cn3)c2)c(F)c1] | ESC1002575 | 101 | 10 | 102 | 1.3 | 6.57 |
| 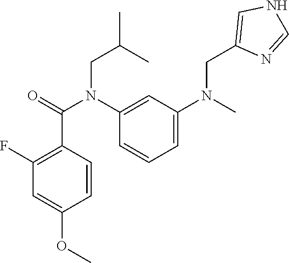 [SMILES: COc1ccc(C(=O)N(CC(C)C)c2cccc(N(C)Cc3c[nH]cn3)c2)c(F)c1] | ESC1002576 | 97 | −3 | 98 | 1.1 | 6.61 |
| 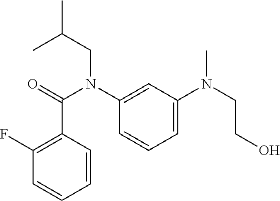 [SMILES: CC(C)CN(C(=O)c1ccccc1F)c1cccc(N(C)CCO)c1] | ESC1002577 | 100 | −1 | 72 | 0.95 | 5.09 |
| 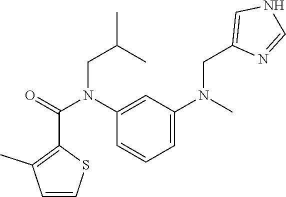 [SMILES: Cc1ccsc1C(=O)N(CC(C)C)c1cccc(N(C)Cc2c[nH]cn2)c1] | ESC1002597 | 96 | 4 | 100 | 1.4 | 6.4 |
| 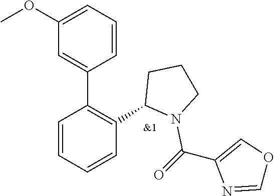 [SMILES: COc1cccc(-c2ccccc2[C@@H]2CCCN2C(=O)c2cocn2)c1] | ESC1002598 | | | 50 | | <4.7 |
| 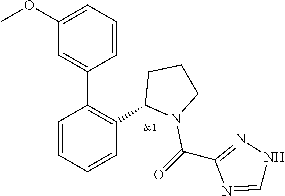 [SMILES: COc1cccc(-c2ccccc2[C@@H]2CCCN2C(=O)c2nc[nH]n2)c1] | ESC1002599 | | | 10 | | <4.7 |
| 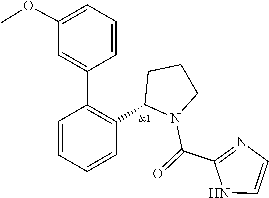 [SMILES: COc1cccc(-c2ccccc2[C@@H]2CCCN2C(=O)c2ncc[nH]2)c1] | ESC1002600 | 86 | 1 | 75 | 1.2 | 5.36 |
| 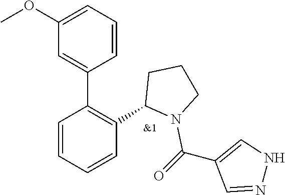 [SMILES: COc1cccc(-c2ccccc2[C@@H]2CCCN2C(=O)c2cn[nH]c2)c1] | ESC1002601 | 115 | −7 | 82 | 0.74 | 5.2 |
| 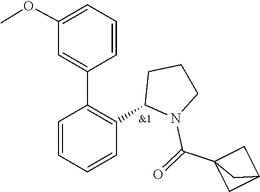 [SMILES: COc1cccc(-c2ccccc2[C@@H]2CCCN2C(=O)C23CC(C2)C3)c1] | ESC1002602 | | | 43 | | <4.7 |
| 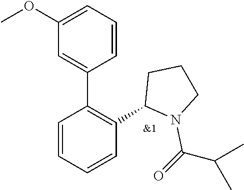 [SMILES: COc1cccc(-c2ccccc2[C@@H]2CCCN2C(=O)C(C)C)c1] | ESC1002603 | | | 21 | | <4.7 |
| 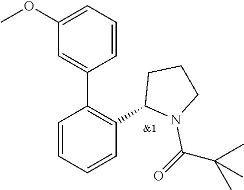 [SMILES: COc1cccc(-c2ccccc2[C@@H]2CCCN2C(=O)C(C)(C)C)c1] | ESC1002604 | | | 20 | | <4.7 |
| 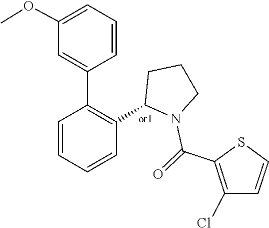 [SMILES: COc1cccc(-c2ccccc2[C@@H]2CCCN2C(=O)c2sccc2Cl)c1] | ESC1002605 | | | 17 | | <4.7 |
| 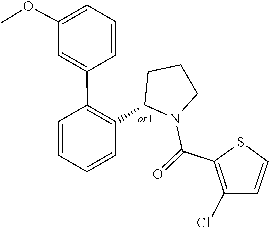 [SMILES: COc1cccc(-c2ccccc2[C@@H]2CCCN2C(=O)c2sccc2Cl)c1] | ESC1002606 | 96 | 0 | 98 | 1.5 | 6.96 |
| 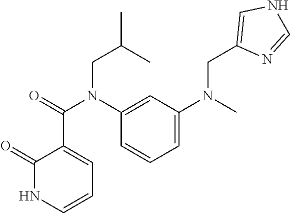 [SMILES: CC(C)CN(C(=O)c1ccc[nH]c1=O)c1cccc(N(C)Cc2c[nH]cn2)c1] | ESC1002614 | | | 30 | | <4.7 |
| 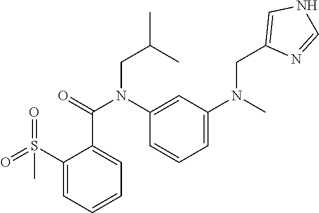 [SMILES: CC(C)CN(C(=O)c1ccccc1S(C)(=O)=O)c1cccc(N(C)Cc2c[nH]cn2)c1] | ESC1002622 | 108 | −6 | 76 | 0.9 | 5.16 |
| 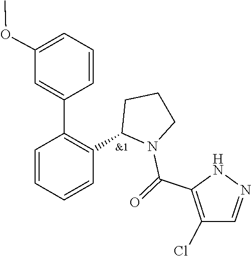 [SMILES: COc1cccc(-c2ccccc2[C@@H]2CCCN2C(=O)c2[nH]ncc2Cl)c1] | ESC1002626 | 91 | −1 | 95 | 1.6 | 6.34 |
| 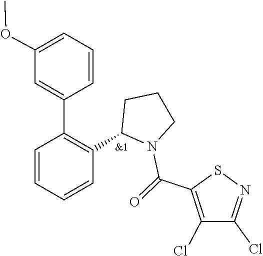 [SMILES: COc1cccc(-c2ccccc2[C@@H]2CCCN2C(=O)c2snc(Cl)c2Cl)c1] | ESC1002627 | | | 5 | | <4.7 |
| 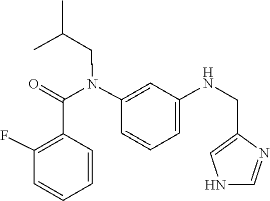 [SMILES: CC(C)CN(C(=O)c1ccccc1F)c1cccc(NCc2c[nH]cn2)c1] | ESC1002631 | 100 | −6 | 64 | 0.94 | 5.03 |
| 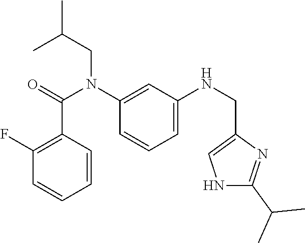 [SMILES: CC(C)CN(C(=O)c1ccccc1F)c1cccc(NCc2c[nH]c(C(C)C)n2)c1] | ESC1002632 | | | 18 | | <4.7 |
| 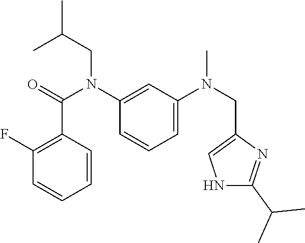 [SMILES: CC(C)CN(C(=O)c1ccccc1F)c1cccc(N(C)Cc2c[nH]c(C(C)C)n2)c1] | ESC1002633 | 100 | −3 | 45 | 0.93 | <4.7 |
| 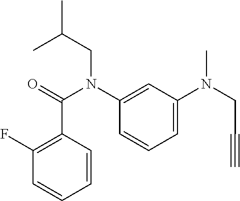 [SMILES: C#CCN(C)c1cccc(N(CC(C)C)C(=O)c2ccccc2F)c1] | ESC1002636 | 101 | −1 | 79 | 0.98 | 5.26 |
| 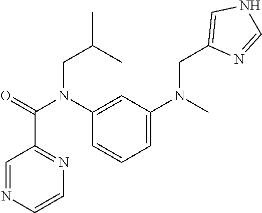 [SMILES: CC(C)CN(C(=O)c1cnccn1)c1cccc(N(C)Cc2c[nH]cn2)c1] | ESC1002656 | | | 45 | | <4.7 |
| 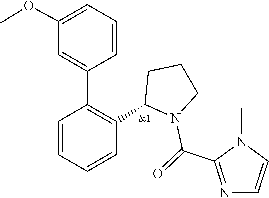 [SMILES: COc1cccc(-c2ccccc2[C@@H]2CCCN2C(=O)c2nccn2C)c1] | ESC1002661 | | | 7 | | <4.7 |
| 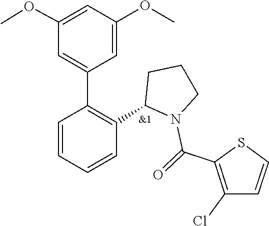 [SMILES: COc1cc(OC)cc(-c2ccccc2[C@@H]2CCCN2C(=O)c2sccc2Cl)c1] | ESC1002662 | 92 | −1 | 92 | 1.2 | 6.22 |
| 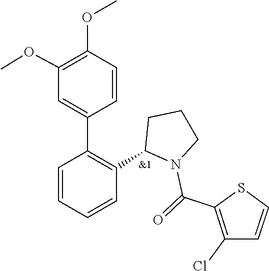 [SMILES: COc1ccc(-c2ccccc2[C@@H]2CCCN2C(=O)c2sccc2Cl)cc1OC] | ESC1002663 | 95 | −8 | 93 | 1.1 | 6.24 |
| 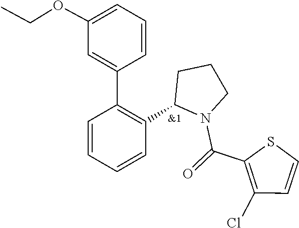 [SMILES: CCOc1cccc(-c2ccccc2[C@@H]2CCCN2C(=O)c2sccc2Cl)c1] | ESC1002664 | 92 | 0 | 92 | 1.2 | 6.38 |
| 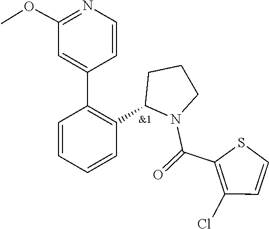 [SMILES: COc1cc(-c2ccccc2[C@@H]2CCCN2C(=O)c2sccc2Cl)ccn1] | ESC1002665 | 97 | −3 | 95 | 1 | 6.22 |
| 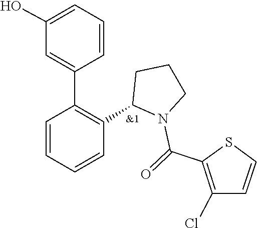 [SMILES: O=C(c1sccc1Cl)N1CCC[C@H]1c1ccccc1-c1cccc(O)c1] | ESC1002666 | 98 | 0 | 99 | 1.1 | 7.23 |
| 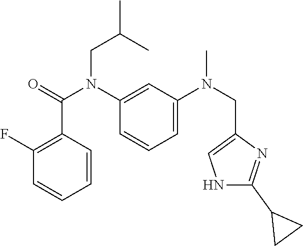 [SMILES: CC(C)CN(C(=O)c1ccccc1F)c1cccc(N(C)Cc2c[nH]c(C3CC3)n2)c1] | ESC1002674 | 80 | −5 | 59 | 1.2 | 5.08 |
| 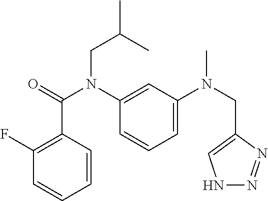 [SMILES: CC(C)CN(C(=O)c1ccccc1F)c1cccc(N(C)Cc2c[nH]nn2)c1] | ESC1002675 | 87 | −2 | 75 | 1.2 | 5.35 |
| 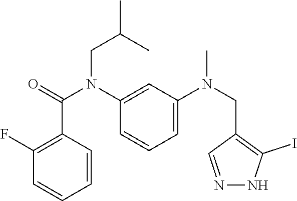 [SMILES: CC(C)CN(C(=O)c1ccccc1F)c1cccc(N(C)Cc2cn[nH]c2I)c1] | ESC1002687 | 100 | −6 | 64 | 1 | 4.96 |
| 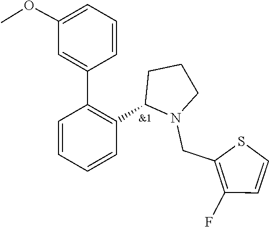 [SMILES: COc1cccc(-c2ccccc2[C@@H]2CCCN2Cc2sccc2F)c1] | ESC1002695 | | | 1 | | <4.7 |
| 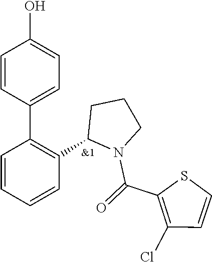 [SMILES: O=C(c1sccc1Cl)N1CCC[C@H]1c1ccccc1-c1ccc(O)cc1] | ESC1002696 | 97 | 1 | 97 | 1.2 | 6.52 |
| 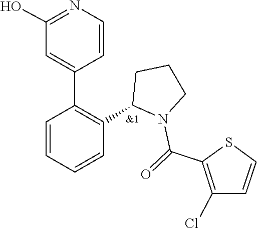 [SMILES: O=C(c1sccc1Cl)N1CCC[C@H]1c1ccccc1-c1ccnc(O)c1] | ESC1002697 | | | 43 | | <4.7 |
| 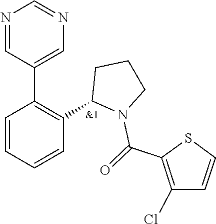 [SMILES: O=C(c1sccc1Cl)N1CCC[C@H]1c1ccccc1-c1cncnc1] | ESC1002698 | 71 | 1 | 59 | 1.1 | 5.13 |
| 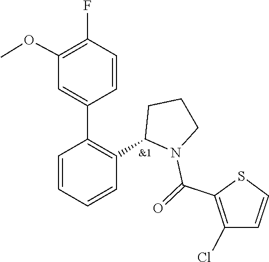 [SMILES: COc1cc(-c2ccccc2[C@@H]2CCCN2C(=O)c2sccc2Cl)ccc1F] | ESC1002699 | 96 | 5 | 97 | 1.2 | 6.58 |
| 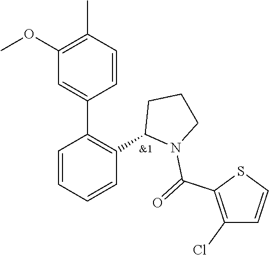 [SMILES: COc1cc(-c2ccccc2[C@@H]2CCCN2C(=O)c2sccc2Cl)ccc1C] | ESC1002700 | 92 | −2 | 93 | 1.4 | 6.4 |
| 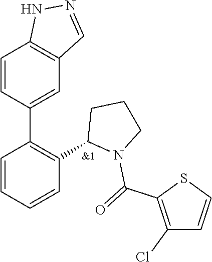 [SMILES: O=C(c1sccc1Cl)N1CCC[C@H]1c1ccccc1-c1ccc2[nH]ncc2c1] | ESC1002701 | 96 | 0 | 99 | 1.2 | 6.54 |
| 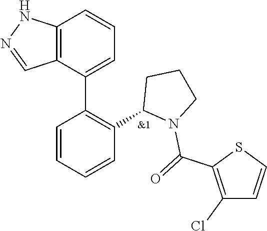 [SMILES: O=C(c1sccc1Cl)N1CCC[C@H]1c1ccccc1-c1cccc2[nH]ncc12] | ESC1002702 | 98 | −9 | 91 | 0.91 | 5.9 |
| 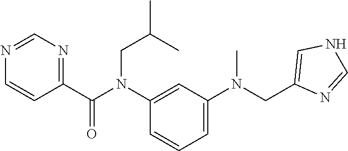 [SMILES: CC(C)CN(C(=O)c1ccncn1)c1cccc(N(C)Cc2c[nH]cn2)c1] | ESC1002703 | 70 | −5 | 52 | 1.3 | 5.06 |
| 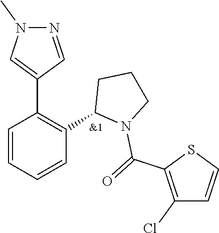 [SMILES: Cn1cc(-c2ccccc2[C@@H]2CCCN2C(=O)c2sccc2Cl)cn1] | ESC1002704 | 103 | −10 | 98 | 1 | 5.97 |
| 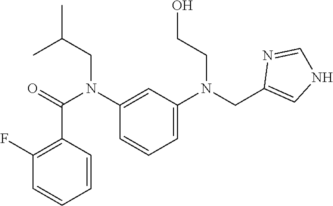 [SMILES: CC(C)CN(C(=O)c1ccccc1F)c1cccc(N(CCO)Cc2c[nH]cn2)c1] | ESC1002723 | 94 | 0 | 81 | 1.1 | 5.29 |
| 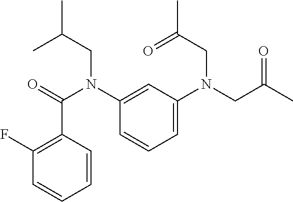 [SMILES: CC(=O)CN(CC(C)=O)c1cccc(N(CC(C)C)C(=O)c2ccccc2F)c1] | ESC1002724 | | | −3 | | <4.7 |
| 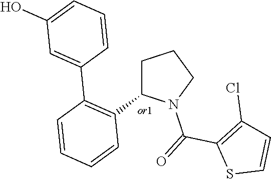 [SMILES: O=C(c1sccc1Cl)N1CCC[C@H]1c1ccccc1-c1cccc(O)c1] | ESC1002754 | | | 20 | | <4.7 |
| 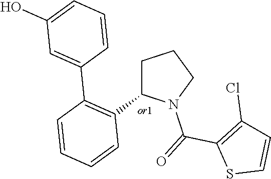 [SMILES: O=C(c1sccc1Cl)N1CCC[C@H]1c1ccccc1-c1cccc(O)c1] | ESC1002755 | 93 | −2 | 95 | 1 | 7.7 |
| 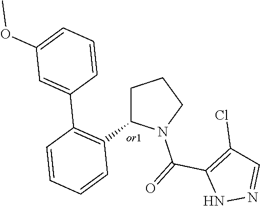 [SMILES: COc1cccc(-c2ccccc2[C@@H]2CCCN2C(=O)c2[nH]ncc2Cl)c1] | ESC1002756 | | | 24 | | <4.7 |
| 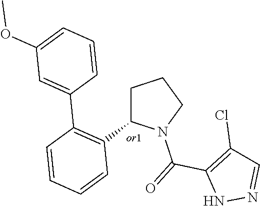 [SMILES: COc1cccc(-c2ccccc2[C@@H]2CCCN2C(=O)c2[nH]ncc2Cl)c1] | ESC1002757 | 95 | −10 | 94 | 0.89 | 6.94 |
| 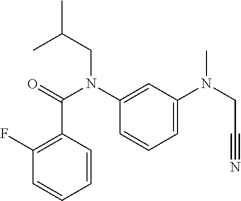 [SMILES: CC(C)CN(C(=O)c1ccccc1F)c1cccc(N(C)CC#N)c1] | ESC1002762 | | | 39 | | <4.7 |
| 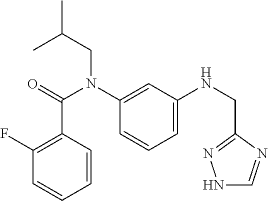 [SMILES: CC(C)CN(C(=O)c1ccccc1F)c1cccc(NCc2nc[nH]n2)c1] | ESC1002763 | | | 8 | | <4.7 |
| 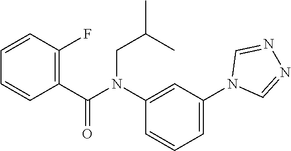 [SMILES: CC(C)CN(C(=O)c1ccccc1F)c1cccc(-n2cnnc2)c1] | ESC1002764 | | | 4 | | <4.7 |
| 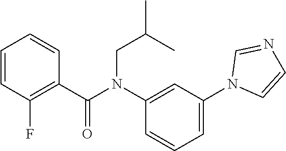 [SMILES: CC(C)CN(C(=O)c1ccccc1F)c1cccc(-n2ccnc2)c1] | ESC1002765 | | | −2 | | <4.7 |
| 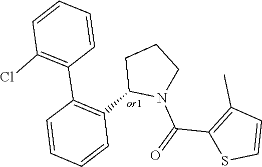 [SMILES: Cc1ccsc1C(=O)N1CCC[C@H]1c1ccccc1-c1ccccc1Cl] | ESC1002766 | | | 14 | | <4.7 |
| 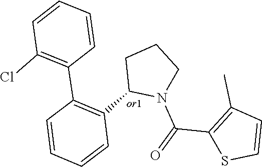 [SMILES: Cc1ccsc1C(=O)N1CCC[C@H]1c1ccccc1-c1ccccc1Cl] | ESC1002767 | 96 | −2 | 96 | 0.98 | 6.81 |
| 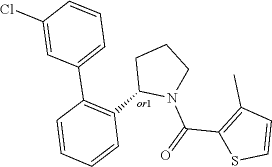 [SMILES: Cc1ccsc1C(=O)N1CCC[C@H]1c1ccccc1-c1cccc(Cl)c1] | ESC1002768 | | | 12 | | <4.7 |
| 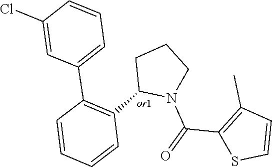 [SMILES: Cc1ccsc1C(=O)N1CCC[C@H]1c1ccccc1-c1cccc(Cl)c1] | ESC1002769 | 93 | −3 | 94 | 1 | 6.72 |
| 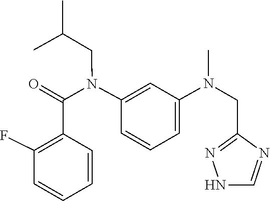 [SMILES: CC(C)CN(C(=O)c1ccccc1F)c1cccc(N(C)Cc2nc[nH]n2)c1] | ESC1002770 | 91 | −4 | 86 | 1.2 | 5.57 |
| 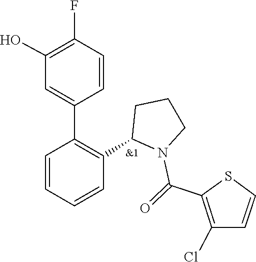 [SMILES: O=C(c1sccc1Cl)N1CCC[C@H]1c1ccccc1-c1ccc(F)c(O)c1] | ESC1002775 | 96 | 0 | 100 | 1.2 | 7.31 |
| 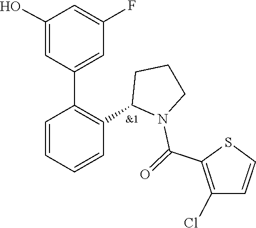 [SMILES: O=C(c1sccc1Cl)N1CCC[C@H]1c1ccccc1-c1cc(O)cc(F)c1] | ESC1002776 | 96 | −1 | 99 | 1 | 7.39 |
| 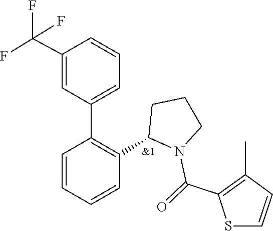 [SMILES: Cc1ccsc1C(=O)N1CCC[C@H]1c1ccccc1-c1cccc(C(F)(F)F)c1] | ESC1002789 | | | 11 | | <4.7 |
| 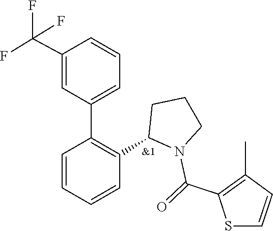 [SMILES: Cc1ccsc1C(=O)N1CCC[C@H]1c1ccccc1-c1cccc(C(F)(F)F)c1] | ESC1002790 | 93 | −1 | 93 | 1.2 | 6.18 |
| 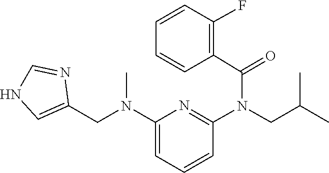 [SMILES: CC(C)CN(C(=O)c1ccccc1F)c1cccc(N(C)Cc2c[nH]cn2)n1] | ESC1002795 | 93 | 0 | 77 | 1.1 | 5.25 |
| 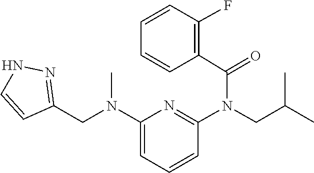 [SMILES: CC(C)CN(C(=O)c1ccccc1F)c1cccc(N(C)Cc2cc[nH]n2)n1] | ESC1002796 | 100 | −4 | 47 | 1.2 | <4.7 |
| 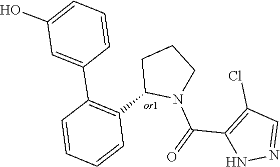 [SMILES: O=C(c1[nH]ncc1Cl)N1CCC[C@H]1c1ccccc1-c1cccc(O)c1] | ESC1002798 | | | 18 | | <4.7 |
| 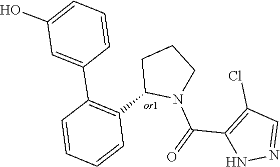 [SMILES: O=C(c1[nH]ncc1Cl)N1CCC[C@H]1c1ccccc1-c1cccc(O)c1] | ESC1002799 | 96 | −3 | 97 | 1.1 | 6.81 |
| 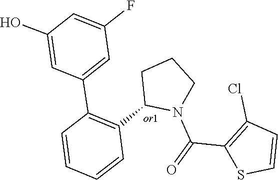 [SMILES: O=C(c1sccc1Cl)N1CCC[C@H]1c1ccccc1-c1cc(O)cc(F)c1] | ESC1002800 | | | 10 | | <4.7 |
| 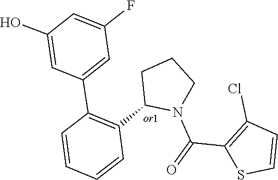 [SMILES: O=C(c1sccc1Cl)N1CCC[C@H]1c1ccccc1-c1cc(O)cc(F)c1] | ESC1002801 | 96 | −1 | 100 | 1.2 | 7.29 |
| 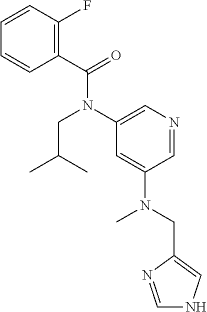 [SMILES: CC(C)CN(C(=O)c1ccccc1F)c1cncc(N(C)Cc2c[nH]cn2)c1] | ESC1002802 | | | 20 | | <4.7 |
| 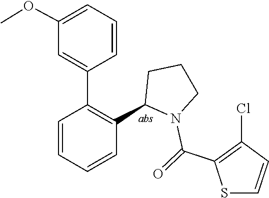 [SMILES: COc1cccc(-c2ccccc2[C@H]2CCCN2C(=O)c2sccc2Cl)c1] | ESC1002804 | | | 25 | | <4.7 |
| 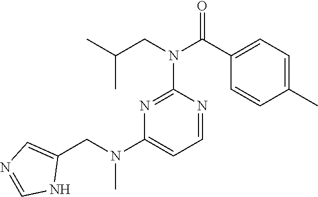 [SMILES: Cc1ccc(C(=O)N(CC(C)C)c2nccc(N(C)Cc3cnc[nH]3)n2)cc1] | ESC1002817 | | | 4 | | <4.7 |
| 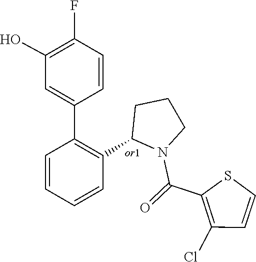 [SMILES: O=C(c1sccc1Cl)N1CCC[C@H]1c1ccccc1-c1ccc(F)c(O)c1] | ESC1002838 | 97 | 0 | 99 | 1.2 | 7.29 |
| 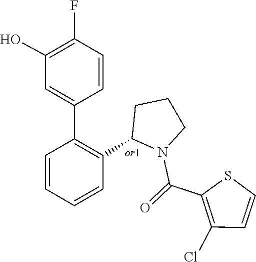 [SMILES: O=C(c1sccc1Cl)N1CCC[C@H]1c1ccccc1-c1ccc(F)c(O)c1] | ESC1002839 | 96 | −4 | 85 | 1.1 | 5.38 |
| 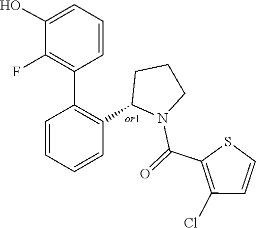 [SMILES: O=C(c1sccc1Cl)N1CCC[C@H]1c1ccccc1-c1cccc(O)c1F] | ESC1002840 | 97 | −4 | 98 | 1.1 | 6.77 |
| 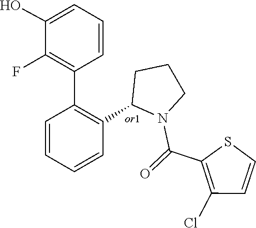 [SMILES: O=C(c1sccc1Cl)N1CCC[C@H]1c1ccccc1-c1cccc(O)c1F] | ESC1002841 | | | 27 | | <4.7 |
| 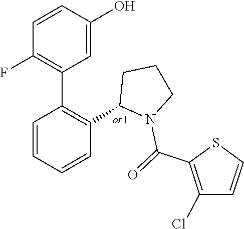 [SMILES: O=C(c1sccc1Cl)N1CCC[C@H]1c1ccccc1-c1cc(O)ccc1F] | ESC1002842 | 98 | −3 | 102 | 1.1 | 7.39 |
| 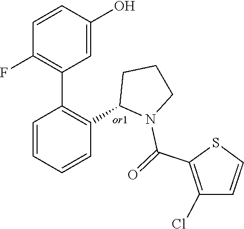 [SMILES: O=C(c1sccc1Cl)N1CCC[C@H]1c1ccccc1-c1cc(O)ccc1F] | ESC1002843 | 100 | −1 | 70 | 1.1 | 4.97 |
| 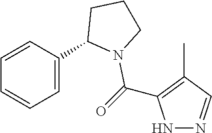 [SMILES: Cc1cn[nH]c1C(=O)N1CCC[C@H]1c1ccccc1] | S1−T1 | | | 26.5 | | <4.7 |
| 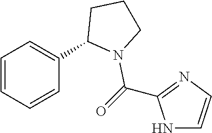 [SMILES: O=C(c1ncc[nH]1)N1CCC[C@H]1c1ccccc1] | S1−T2 | | | 6.4 | | <4.7 |
| 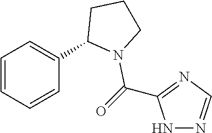 [SMILES: O=C(c1ncn[nH]1)N1CCC[C@H]1c1ccccc1] | S1−T3 | | | 7.1 | | <4.7 |
| 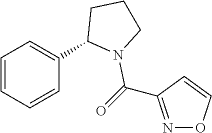 [SMILES: O=C(c1ccon1)N1CCC[C@H]1c1ccccc1] | S1−T5 | | | 27.5 | | <4.7 |
| 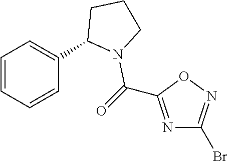 [SMILES: O=C(c1nc(Br)no1)N1CCC[C@H]1c1ccccc1] | S1−T6 | | | 20.5 | | <4.7 |
| 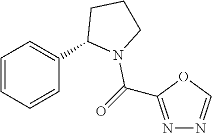 [SMILES: O=C(c1nnco1)N1CCC[C@H]1c1ccccc1] | S1−T7 | | | 10.4 | | <4.7 |
| 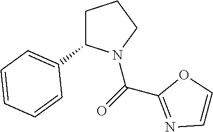 [SMILES: O=C(c1ncco1)N1CCC[C@H]1c1ccccc1] | S1−T9 | | | 26.6 | | <4.7 |
| 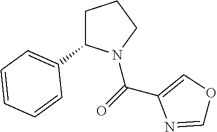 [SMILES: O=C(c1cocn1)N1CCC[C@H]1c1ccccc1] | S1−T10 | | | 20.5 | | <4.7 |
| 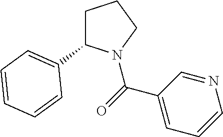 [SMILES: O=C(c1cccnc1)N1CCC[C@H]1c1ccccc1] | S1−T12 | | | 9.1 | | <4.7 |
| 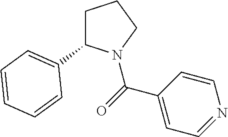 [SMILES: O=C(c1ccncc1)N1CCC[C@H]1c1ccccc1] | S1−T13 | | | 22.3 | | <4.7 |
| 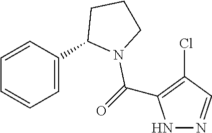 [SMILES: O=C(c1[nH]ncc1Cl)N1CCC[C@H]1c1ccccc1] | S1−T14 | | | 20.8 | | <4.7 |
| 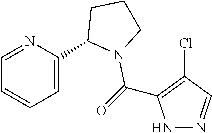 [SMILES: O=C(c1[nH]ncc1Cl)N1CCC[C@H]1c1ccccn1] | S1−T17 | | | 17.5 | | <4.7 |
| 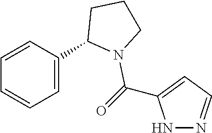 [SMILES: O=C(c1ccn[nH]1)N1CCC[C@H]1c1ccccc1] | S1−T22 | | | 28.3 | | <4.7 |
| 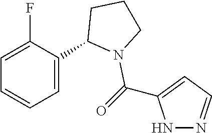 [SMILES: O=C(c1ccn[nH]1)N1CCC[C@H]1c1ccccc1F] | S1−T24 | | | 17.1 | | <4.7 |
| 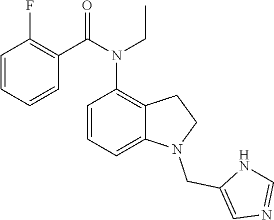 [SMILES: CCN(C(=O)c1ccccc1F)c1cccc2c1CCN2Cc1cnc[nH]1] | S2−T1 | | | 10.2 | | <4.7 |
| 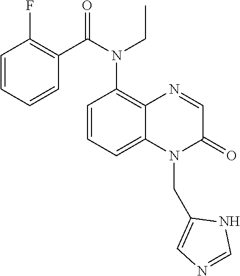 [SMILES: CCN(C(=O)c1ccccc1F)c1cccc2c1ncc(=O)n2Cc1cnc[nH]1] | S2−T2 | | | 4.8 | | <4.7 |
| 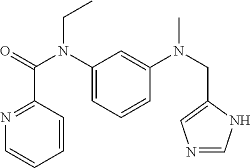 [SMILES: CCN(C(=O)c1ccccn1)c1cccc(N(C)Cc2cnc[nH]2)c1] | S2−T3 | | | 20.3 | | <4.7 |
| 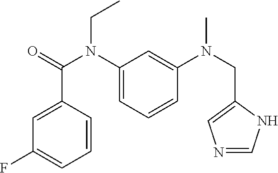 [SMILES: CCN(C(=O)c1cccc(F)c1)c1cccc(N(C)Cc2cnc[nH]2)c1] | S2−T5 | | | 18.4 | | <4.7 |
| 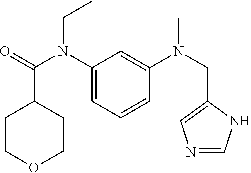 [SMILES: CCN(C(=O)C1CCOCC1)c1cccc(N(C)Cc2cnc[nH]2)c1] | S2−T6 | | | 18.4 | | <4.7 |
| 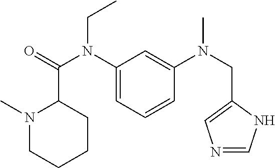 [SMILES: CCN(C(=O)C1CCCCN1C)c1cccc(N(C)Cc2cnc[nH]2)c1] | S2−T7 | | | 27.9 | | <4.7 |
| 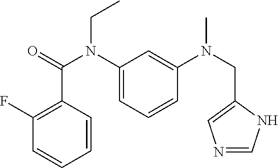 [SMILES: CCN(C(=O)c1ccccc1F)c1cccc(N(C)Cc2cnc[nH]2)c1] | S2−T8 | | | 18.4 | | <4.7 |

TABLE 7
Cytotoxicity and viability of compounds
in mtsABAD HEK293 cells.

| | | A) LDH % | B) Alamar Blue % |
|---|---|---|---|
| | | Toxicity | Viability |
| Compound ID | MW | (50 μM Dose) | (50 μM Dose) |
| ESC1002089-01 | 377.5 | 5.398 | 83.064 |
| ESC1002090-01 | 348.5 | 5.162 | 102.627 |
| ESC1002091-01 | 337.4 | 5.395 | 84.920 |
| ESC1002092-01 | 337.4 | 4.240 | 80.606 |
| ESC1002119-01 | 376.42 | 4.780 | 79.180 |
| ESC1002120-01 | 372.45 | 4.611 | 81.743 |
| ESC1002161-01 | 378.49 | 4.783 | 92.566 |
| ESC1002162-01 | 378.49 | 6.843 | 83.498 |
| ESC1002170-01 | 264.33 | 5.192 | 79.141 |
| ESC1002163-01 | 382.44 | 4.975 | 78.775 |
| ESC1002164-01 | 440.52 | 5.093 | 77.951 |
| ESC1002165-01 | 328.46 | 4.074 | 88.291 |
| ESC1002166-01 | 368.52 | 4.554 | 82.237 |
| ESC1002167-01 | 376.5 | 4.492 | 79.269 |
| ESC1002168-01 | 390.53 | 4.285 | 82.648 |
| ESC1002169-01 | 362.47 | 4.554 | 84.229 |
| ESC1002171-01 | 300.4 | 5.933 | 86.843 |
| ESC1002172-01 | 412.53 | 5.472 | 85.337 |
| ESC1002183-01 | 396.92 | 5.082 | 89.373 |
| ESC1002182-01 | 392.5 | 4.733 | 87.643 |
| ESC1002199-01 | 430.19 | 5.028 | 89.524 |
| ESC1002200-01 | 430.19 | 5.136 | 79.220 |
| ESC1002201-01 | 416.18 | 5.842 | 80.481 |
| ESC1002202-01 | 430.19 | 2.542 | 81.531 |
| ESC1002203-01 | 430.19 | 6.638 | 87.491 |
| ESC1002204-01 | 394.2 | 5.048 | 88.836 |
| ESC1002205-01 | 380.2 | 5.840 | 95.933 |
| ESC1002206-01 | 380.2 | 5.918 | 95.609 |
| ESC1002205-02 | 416.18 | 5.243 | 89.638 |
| ESC1002208-01 | 433.14 | 20.084 | 65.577 |
| ESC1002209-01 | 427.18 | 5.698 | 80.514 |
| ESC1002210-01 | 427.18 | 5.509 | 93.200 |
| ESC1002211-01 | 391.21 | 5.501 | 82.553 |
| ESC1002212-01 | 407.2 | 5.576 | 94.278 |
| ESC1002213-01 | 421.2 | 8.010 | 81.860 |
| ESC1002214-02 | 430.2 | 5.902 | 82.439 |
| ESC1002234-01 | 352.41 | 4.81 | 86.98 |
| ESC1002235-01 | 328.46 | 5.54 | 84.09 |
| ESC1002236-01 | 406.5 | 3.94 | 82.69 |
| ESC1002265-01 | 392.48 | 4.52 | 80.52 |
| ESC1002267-01 | 410.49 | 4.81 | 89.57 |
| ESC1002266-01 | 382.44 | 5.22 | 89.86 |
| ESC1002314-01 | 415.2 | 5.98 | 87.10 |
| ESC1002315-01 | 431.1 | 5.91 | 84.77 |
| ESC1002316-01 | 372.1 | 3.69 | 80.39 |
| ESC1002317-01 | 418.2 | 4.58 | 83.39 |
| ESC1002318-01 | 432.2 | 2.55 | 85.76 |
| ESC1002319-01 | 425.1 | 5.17 | 82.10 |
| ESC1002320-01 | 415.1 | 5.12 | 83.37 |
| ESC1002321-01 | 381.1 | 5.98 | 84.52 |
| ESC1002322-01 | 425.1 | 6.93 | 89.24 |
| ESC1002323-01 | 361.2 | 5.03 | 96.19 |
| ESC1002324-01 | 381.1 | 5.04 | 85.64 |
| ESC1002325-01 | 377.1 | 4.36 | 89.12 |
| ESC1002326-01 | 407.2 | 5.05 | 88.92 |
| ESC1002327-02 | 449.1 | 4.61 | 90.36 |
| ESC1002332-02 | 377.49 | 3.52 | 87.68 |
| ESC1002331-01 | 377.49 | 5.88 | 88.56 |
| ESC1002334-01 | 395.48 | 5.17 | 92.96 |
| ESC1002340-01 | 415.06 | 5.33 | 94.26 |
| ESC1002338-01 | 377.14 | 4.64 | 82.98 |
| ESC1002341-01 | 421.18 | 5.13 | 96.72 |
| ESC1002339-01 | 375.17 | 4.39 | 82.52 |
| ESC1002337-01 | 361.15 | 4.10 | 85.13 |
| ESC1002342-01 | 378.45 | 4.76 | 85.04 |
| ESC1002344-01 | 523.2 | 5.75 | 90.97 |

A) Percentage cytotoxicity of mtsABAD HEK293 cells treated with 50 μM compound as measured by lactate dehydrogenase assay.
B) Percentage of viable mtsABAD HEK293 cells treated with 50 μM compound as measured by Alamar Blue assay.

1. A compound of formula (I) or (II): 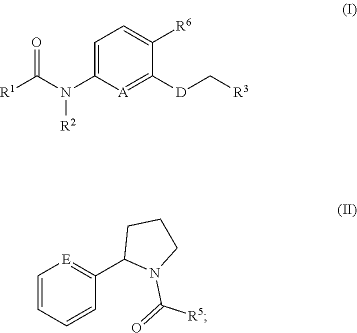
wherein R¹ is any one selected from the group consisting of C₆-C₁₀aryl, C₃-C₅heteroaryl, C₁₀-C₁₆biaryl, C₆-C₁₄biheteroaryl, C₅-C₈cycloalkyl, C₃-C₇heterocycloalkyl, C₁-C₆alkyl, C₆-C₁₀arylC₁-C₃alkyl, C₁₀-C₁₆biarylC₁-C₃alkyl and C₃-C₈cycloalkylC₁-C₃alkyl optionally substituted one or more times with any one or a combination selected from the group consisting of halo, C₁-C₄haloalkyl, C₁-C₄alkyl, C₁-C₄alkoxy, C₁-C₄haloalkoxy, hydroxy and methylsulfonyl;
R² is any one selected from the group consisting of C₁-C₄alkyl, C₃-C₅cycloalkyl, C₃-C₅cycloalkylC₁-C₃alkyl, C₁-C₄alkylether, and di(C₁₋₄alkyl)aminoC₁₋₃alkyl;
A is CH or N;
R³ is any one selected from the group consisting of C₂-C₅heteroaryl optionally substituted one or more times with any one or a combination selected from the group consisting of C₁-C₄alkyl, C₃-C₅cycloalkyl, halo, hydroxy and C₁₋₃alkoxy; ethyne, hydroxymethyl, amido, N-methylamido and cyano;
D is NR⁷ or O, wherein R⁷ is C₁₋₃alkyl or C₁₋₃alkylol;
R⁶ is H; or
R⁶ and D together form a 5 or 6 membered heterocycle optionally substituted one or more times with any one or a combination selected from the group consisting of oxo, halo, C₁₋₃alkyl, C₁₋₃haloalkyl, hydroxy and C₁₋₃alkoxy;
E is CR⁴ or N;
R⁴ is any one selected from the group consisting of C₆aryl, C₃-C₇heteroaryl, C₆-C₁₄biheteroaryl, H, halo, C₁₋₆alkyl, C₁₋₆haloalkyl, C₁₋₆alkoxy, wherein the C₆aryl, C₃-C₇heteroaryl and C₆-C₁₄biheteroaryl are optionally substituted one or more times with any one or a combination selected from the group consisting of hydroxy, C₁-C₄alkoxy, C₁-C₄haloalkoxy, halo, C₁-C₄haloalkyl, C₁-C₄alkyl, C₃-C₅cycloalkyl, C₄-C₅heterocycloalkyl, C₁-C₄alkanol, cyano, methane sulfonate and dimethylamido; and
R⁵ is any one selected from the group consisting of C₂-C₅heteroaryl, C₆aryl, C₃₋₅cycloalkyl and C₁₋₄alkyl, wherein the C₂-C₅heteroaryl, C₆aryl and C₃₋₅cycloalkyl are each optionally substituted one or more times with any one or a combination selected from the group consisting of halo and C₁-C₄alkyl.

2. The compound of claim 1, wherein R¹ is any one selected from the group consisting of C₆-C₁₀aryl, C₃-C₅heteroaryl, C₁₀-C₁₆biaryl, C₅-C₈cycloalkyl, C₃-C₆alkyl and C₆-C₁₀arylC₁-C₃alkyl, optionally substituted one or more times with any one or a combination selected from the group consisting of halo, C₁-C₄haloalkyl, C₁-C₄alkyl, C₁-C₄alkoxy, hydroxy and methylsulfonyl.

3. The compound of claim 1, wherein R¹ is any one selected from the group consisting of C₆-C₁₀aryl, C₃-C₅heteroaryl, C₅-C₈cycloalkyl and C₃-C₆alkyl, optionally substituted one or more times with any one or a combination selected from the group consisting of halo, C₁-C₄haloalkyl, C₁-C₄alkyl, C₁-C₄alkoxy and hydroxy.

4. The compound of claim 1, wherein R¹ is any one selected from the group consisting of phenyl, thiophenyl, pyridinyl, cyclohexyl and iso-propyl, optionally substituted one or more times with any one or a combination selected from the group consisting of fluoro, chloro, trifluoromethyl, methyl, ethyl and methoxy.

5. The compound of claim 1, wherein R² is any one selected from the group consisting of C₁-C₄alkyl and C₃-C₅cycloalkylC₁-C₃alkyl.

6. The compound of claim 1, wherein R² is any one selected from the group consisting of iso-butyl, iso-propyl, ethyl, n-butyl, dimethylpropyl, cyclobutylmethyl and cyclopropylmethyl.

7. The compound of claim 1, wherein A is CH.

8. The compound of claim 1, wherein R³ is an optionally substituted C₂-C₅heteroaryl comprising nitrogen and/or sulfur as heteroatoms;
optionally wherein R³ comprises nitrogen as the only heteroatom.

9. (canceled)

10. The compound of claim 1, wherein R³ is a C₃heteroaryl, optionally substituted one or more times with methyl.

11. The compound of claim 1, wherein R³ is any one selected from the group consisting of imidazolyl, 1-methylimidazolyl, 2-methylimidazolyl, 4-methylimidazolyl and pyrazolyl.

12. The compound of claim 1, wherein R³ is any one selected from the group consisting of imidazol-5-yl, 1-methylimidazol-2-yl, 1-methylimidazol-4-yl, 2-methylimidazol-5-yl, 4-methylimidazol-5-yl and pyrazol-5-yl.

13. The compound of claim 1, wherein D is NCH₃ and R⁶ is H.

14. The compound of claim 1, wherein E is CR⁴.

15. The compound of claim 14, wherein R⁴ is any one selected from the group consisting of phenyl and C₃-C₇heteroaryl optionally substituted one or more times with any one or a combination selected from the group consisting of hydroxy, C₁-C₂alkoxy, C₁-C₂haloalkoxy, halo, halomethyl, C₁-C₃alkyl, C₁-C₃cycloalkyl, C₄-C₅heterocycloalkyl, C₁-C₂alkanol and cyano.

16. The compound of claim 14, wherein R⁴ is any one selected from the group consisting of phenyl, pyrazolyl, pyridinyl and indazolyl optionally substituted one or more times with any one or a combination selected from the group consisting of hydroxy, fluoro, chloro, methyl, trifluoromethyl, trifluoromethoxy, methoxy, ethoxy, propyl, morpholino, tetrahydropyranyl, propanol and cyano.

17. The compound of claim 1, wherein R⁵ is a C₂-C₅heteroaryl and comprises one or more sulfur and/or nitrogen heteroatoms.

18. The compound of claim 1, wherein R⁵ is any one selected from the group consisting of thiophenyl, pyrazolyl, thiazolyl and imidazolyl, optionally substituted one or more times with any one or a combination selected from the group consisting of fluoro, chloro and methyl;
optionally wherein R⁵ is any one selected from the group consisting of thiophen-2-yl, pyrazol-5-yl, pyrazol-4-yl, thiazol-5-yl and imidazol-2-yl, optionally substituted one or more times with any one or a combination selected from the group consisting of fluoro, chloro and methyl.

19. (canceled)

20. A composition comprising the compound of claim 1 and one or more pharmaceutically acceptable excipients.

21-23. (canceled)

24. A method of inhibiting ABAD activity in a subject, the method comprising administering an effective amount of a compound of claim 1 to the subject.

25. A method of treatment comprising administering an effective amount of a compound of claim 1 to a subject:
optionally wherein the method is for the treatment or prophylaxis of dementia or cancer.

26. (canceled)
